(19) United States
(12) Patent Application Publication
(10) Pub. No.: US 2026/0144808 A1
(43) Pub. Date: May 28, 2026

(54) TREATMENT OF REPEAT EXPANSION DISEASE

(71) Applicant: The Johns Hopkins University, Baltimore, MD (US)

(72) Inventors: Jiou Wang, Baltimore, MD (US); Yang Liu, Baltimore, MD (US)

(21) Appl. No.: 19/103,458

(22) PCT Filed: Aug. 17, 2023
(86) PCT No.: PCT/US2023/072348

§ 371 Date: Feb. 12, 2025

Related U.S. Application Data
(60) Provisional application No. 63/399,741, filed on Aug. 22, 2022.

(51) Int. Cl. A61K 31/706; A61P 25/28

(52) U.S. Cl. CPC A61K 31/706; A61P 25/28

(57) ABSTRACT

Provided herein is technology relating to treatment of diseases caused by hexanucleotide repeat expansion in the C9orf72 gene and particularly, but not exclusively, to methods of treating disease by decreasing the activity of death domain-associated protein (DAXX) and/or normalizing histone modifications and chromatin structure.

This application claims priority to U.S. provisional patent application Ser. No. 63/399,741, filed Aug. 22, 2022, which is incorporated herein by reference in its entirety.

### STATEMENT REGARDING FEDERALLY SPONSORED RESEARCH OR DEVELOPMENT

This invention was made with government support under NS089616 and NS128494 awarded by National Institutes of Health. The government has certain rights in the invention.

### SEQUENCE LISTING

The text of the computer readable sequence listing filed herewith, titled “JHU-40902-601_SQL”, created Aug. 16, 2023, having a file size of 30,092 bytes, is hereby incorporated by reference in its entirety.

### FIELD

Provided herein is technology relating to treatment of diseases caused by hexanucleotide repeat expansion in the C9orf72 gene and particularly, but not exclusively, to methods of treating disease by decreasing the activity of death domain-associated protein (DAXX) and/or normalizing histone modifications and chromatin structure throughout the genome.

### BACKGROUND

Nucleotide repeat elements, including microsatellites or short tandem repeats, are common in eukaryotic genomes (T6th et al., 2000). Over 50 different types of genetic disorders, primarily neurological and neuromuscular, have been linked to expanded short nucleotide repeats (Malik et al., 2021). However, understanding the native functions of these repeat elements and their roles in human diseases is still at an early stage.

In normal human populations, a non-coding region of the C9orf72 gene typically comprises two to twenty-five GGGGCC (G4C2; SEQ ID NO: 32) repeats. However, some human disease states result from a process of hexanucleotide repeat expansion (“HRE”) that can produce several hundreds to thousands of units of the GGGGCC (SEQ ID NO: 32) sequence. In particular, HRE associated with the C9orf72 gene (“C9HRE”) is the most common genetic cause of amyotrophic lateral sclerosis (ALS), which is characterized by motor neuron neurodegeneration, and of frontotemporal dementia (FTD), which affects the frontal and temporal lobes of the brain (DeJesus-Hernandez et al., 2011: Renton et al., 2011). In addition, genetic evidence suggests that the C9orf72 HRE also contributes to Alzheimer's disease (Majounie et al., 2012), Huntington's disease (Hensman Moss et al., 2014), and other neurological conditions, including multiple system atrophy (Goldman et al., 2014), depressive pseudodementia (Bieniek et al., 2014), and bipolar disorder (Meisler et al., 2013). However, the pathogenic mechanisms by which the HRE mutation leads to neurodegeneration remain a focus of investigation for the relevant neurodegenerative diseases.

Attempts have been made to explain the etiology of C9orf72 HRE-linked neurodegeneration according to several non-mutually exclusive hypotheses, including loss of C9orf72 functions, aberrant repeat-containing RNAs, and repeat-associated non-ATG-dependent (RAN) translation (Balendra and Isaacs, 2018). In the proposed loss-of-function disease mechanisms, both C9orf72 RNA and protein levels are reduced in patient cells and tissues; however, the regulation of C9orf72 expression under physiological and pathological conditions is not yet fully understood. C9orf72 is a DENN domain-containing protein that functions in several organelles (e.g., lysosomes, mitochondria) and processes (e.g., autophagy) (Amick et al., 2016; Sellier et al., 2016; Sullivan et al., 2016; Ugolino et al., 2016; Wang et al., 2021; Yang et al., 2016). The deficiency in C9orf72 decreases the fitness of human patient-derived motor neurons or compromises immune function in animal models (Burberry et al., 2016; O'Rourke et al., 2016: Shi et al., 2018). In the proposed gain-of-function disease mechanisms, the repeat-containing RNAs sequester RNA-binding proteins (Donnelly et al., 2013; Haeusler et al., 2014), and the RAN translation generates poly-dipeptide repeats (Ash et al., 2013; Mori et al., 2013; Zu et al., 2013), thus leading to RNA and protein toxicity. Loss of C9orf72 functions synergizes with gain-of-function mechanisms in models of C9orf72 HRE-linked diseases (Zhu et al., 2020). Unlike the extensive studies on the C9orf72 HRE RNA and its translational products, the pathogenic mechanisms arising from the repeat-containing DNA remain poorly understood, and it is unclear how the expanded repeat DNA and its protein partners contribute to either loss- or gain-of-function disease mechanisms.

### SUMMARY

In contrast to these previous hypotheses forwarded for the etiology of C9orf72 HRE-linked neurodegeneration, the present technology is based on the results of experiments that indicated that chromatin structure and epigenetic modification at the C9orf72 HRE are associated with (e.g., cause) neurodegeneration. In particular, experiments conducted during the development of the technology described herein identified a DNA-binding protein, DAXX, that recognizes the C9orf72 HRE DNA and regulates global chromatin structure and epigenetic modification, and regulates the transcription of the C9orf72 gene. The HRE mutations induce a nuclear accumulation of DAXX, which undergoes liquid-liquid phase separation (LLPS), a process that drives local and global changes in chromatin structure and epigenetic modification. The results of these experiments identified a stress-inducible feature of C9orf72 gene expression, which is a biologically significant stress-responsive mechanism that is lost in C9orf72 HRE patient cells in a DAXX-dependent manner. The data produced during these experiments further revealed mechanisms by which DAXX and its condensates shape genomic landscapes through chromatin remodeling and epigenetic modifications and illustrate pathogenic cascades initiated from the HRE DNA that affect both loss-of-function and gain-of-function disease processes.

Accordingly, embodiments of the technology relate to methods of treating a subject having a neurodegenerative disease. In some embodiments, methods comprise administering an effective amount of a composition that decreases histone methylation to the subject and/or administering an effective amount of a composition that increases histone acetylation to the subject.

In some embodiments, the subject has amyotrophic lateral sclerosis (ALS). In some embodiments, the subject has frontotemporal degeneration (FTD). In some embodiments, the subject has Alzheimer's disease, Huntington's disease, multiple system atrophy, depressive pseudodementia, or bipolar disorder. In some embodiments, the subject does not have cancer, does not have a tumor, and/or has not been treated for cancer. In some embodiments, the subject does not have acute promyelocytic leukemia and/or has not been treated for acute promyelocytic leukemia.

In some embodiments, the subject is a mammal (e.g., a human).

In some embodiments, methods further comprise detecting increased DAXX amount or activity, histone hypermethylation, and/or histone hypoacetylation in a sample from the subject. In some embodiments, methods further comprise detecting a hexanucleotide repeat expansion in the C9orf72 gene of the subject. In some embodiments, methods comprise detecting abnormal global chromatin structure and/or epigenetic modification of a subject's genome (e.g., by testing a sample from the subject).

In some embodiments, the composition that decreases histone methylation comprises 5-aza-2′-deoxycytidine (decitabine). In some embodiments, the composition that increases histone acetylation to the subject comprises sodium phenylbutyrate. In some embodiments, the composition that decreases histone methylation comprises a DAXX inhibitor. In some embodiments, the composition that increases histone acetylation to the subject comprises a DAXX inhibitor.

In some embodiments, the subject comprises a cell comprising a hexanucleotide repeat in chromosome 9. In some embodiments, the subject comprises a cell comprising a hexanucleotide repeat at a C9orf72 locus. In some embodiments, the subject comprises a cell comprising one or more GGGGCC repeats at a C9orf72 locus.

In some embodiments, the subject is identified as being in need of treatment by detecting the presence and/or amount of a biomarker and/or by detecting a symptom. For example, in some embodiments, the subject has one or more of increased DAXX amount or concentration, increased DAXX activity, abnormal global (e.g., genome-wide) chromatin structure and/or epigenetic modification, increased ATRX amount or concentration, increased ATRX activity, increased SUV9H1 amount or concentration, increased SUV9H1 activity, increased quantity and/or size of ATRX granules in nuclei, ATRX co-condensed with DAXX, increased PML nuclear bodies (PML-NB), increased nuclear localization of HDAC1. HDAC1 co-localized with DAXX, a hexanucleotide (GGGGCC) repeat expansion in the C9orf72 gene, an RNA comprising a hexanucleotide (GGGGCC) repeat expansion, an RNA comprising a G-quadruplex, histone hypermethylation, histone hypomethylation, increased amount or concentration of II3K9me3, decreased amount or concentration of 113K27ac, a DAXX condensate, aberrant distribution pattern of DAXX throughout nuclei, DAXX accumulation at a G4C2 repeat locus, DAXX accumulation at C9orf72, DAXX granules, increased H3K9me3 occupancy at the C9V2 promoter, decreased H3K27ac occupancy at the C9V2 promoter, decreased RNA polymerase IT occupancy at the V2 promoter, increased toxic peptides, decreased chromatin accessibility at the C9V2 promoter, reduced amount or concentration of V2 transcript, increased compactness of chromosomes, global loss of chromatin accessibility in C9orf72, genome-wide DNA hypermethylation, altered epigenetic histone protein modifications, transcriptional dysregulation, increased and/or aberrant formation of sub-TADs at the C9orf72 promoter region, and/or changes in chromatin structure and epigenetic modifications.

In some embodiments, the composition that decreases histone methylation comprises a compound comprising a structure according to:

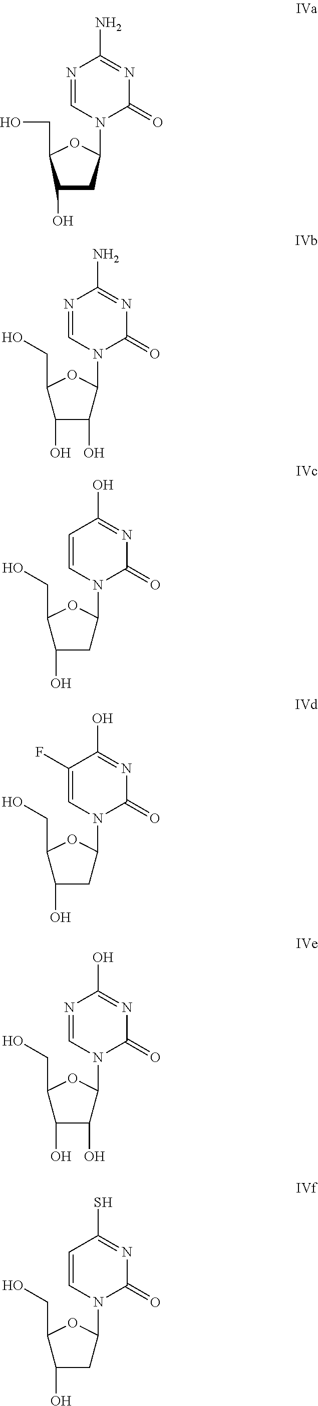

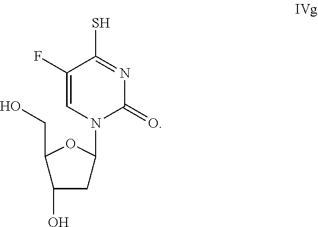 [SMILES: O=c1nc(S)c(F)cn1C1CC(O)C(CO)O1]

In some embodiments, the composition that increases histone acetylation to the subject comprises a compound comprising a structure according to:

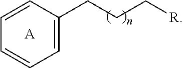

In some embodiments, A comprises one of

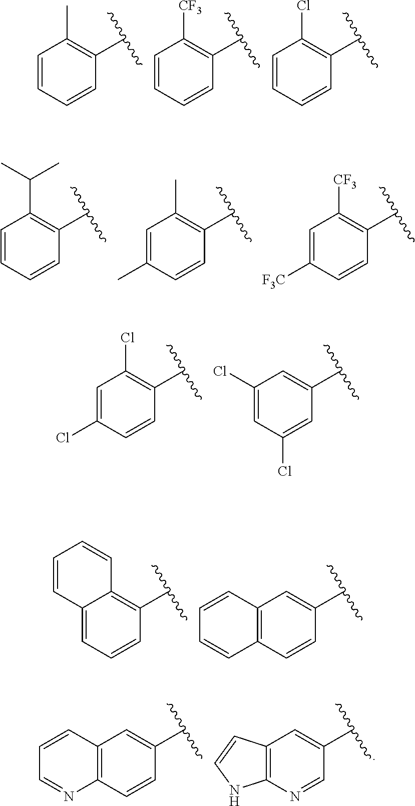

In some embodiments, n=0, 1, 2 3, 4, or 5. In some embodiments, R comprises CO₂Na or one of

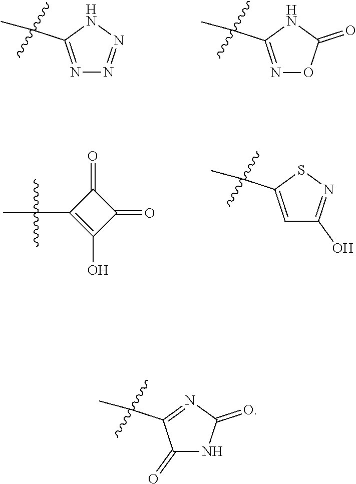 [SMILES: *c1nnn[nH]1 ; *c1noc(=O)[nH]1 ; *c1c(O)c(=O)c1=O ; *c1cc(O)ns1 ; *C1=NC(=O)NC1=O]

In some embodiments, methods further comprise detecting normal histone methylation and/or normal histone acetylation in the subject after said administering. In some embodiments, methods further comprise detecting a decrease in histone methylation relative to pre-treatment amount of histone methylation and/or an increase in histone acetylation relative to pre-treatment amount of histone acetylation in the subject after said administering. In some embodiments, methods further comprise detecting a decrease in DAXX amount or activity relative to pre-treatment amount of DAXX amount or activity in the subject after said administering. In some embodiments, method comprise detecting a normalized global chromatin structure and/or epigenetic modification after said administering relative to an abnormal global chromatin structure and/or epigenetic modification prior to said administering.

In some embodiments, methods further comprise administering a second effective amount of a composition that decreases histone methylation to the subject and/or administering an effective amount of a composition that increases histone acetylation to the subject.

In addition, embodiments of the technology provide compounds (e.g., for treatment of a patient having a neurodegenerative disease). In some embodiments, the technology described herein provides a compound comprising a structure according to one of.

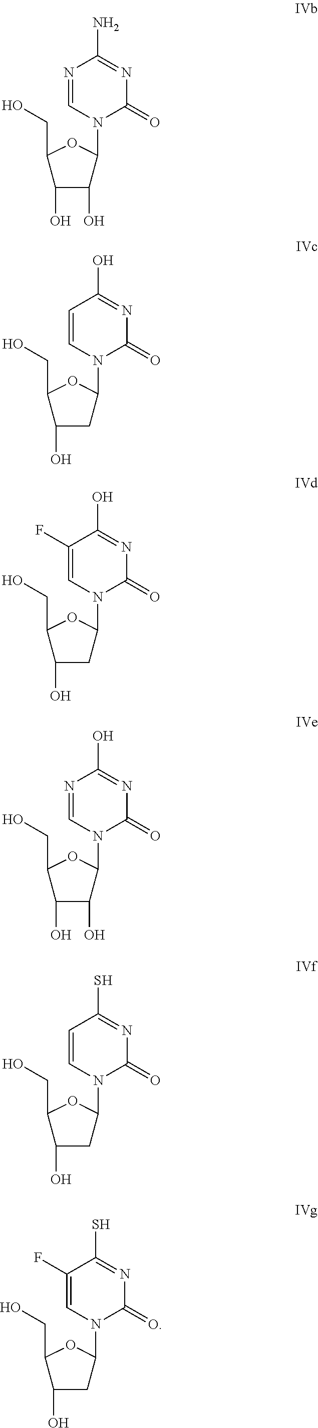

In some embodiments, the technology provides a composition comprising a compound shown above and/or described herein. In some embodiments, the technology provides a pharmaceutical composition comprising a compound shown above and/or described herein. In some embodiments, the pharmaceutical composition is formulated for oral administration.

In some embodiments, the technology described herein provides a compound comprising a structure according to:

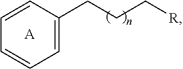

wherein A comprises one of

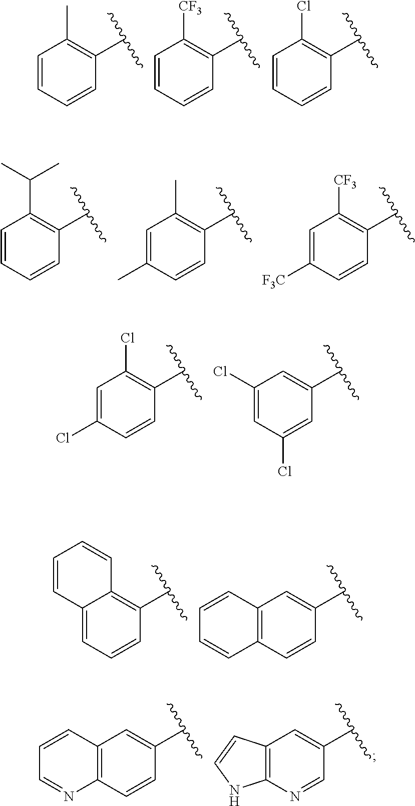

n=0, 1, 2 3, 4, or 5; and/or R comprises CO₂Na or one of:

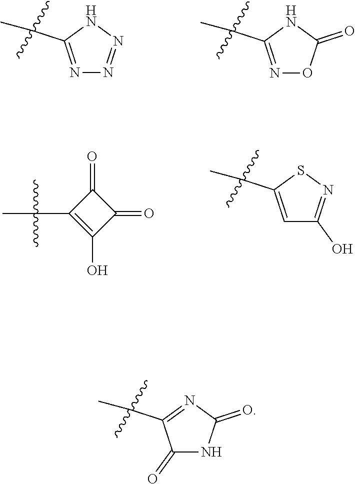 [SMILES: *c1nnn[nH]1 ; *c1noc(=O)[nH]1 ; *c1c(O)c(=O)c1=O ; *c1cc(O)ns1 ; *C1=NC(=O)NC1=O]

In some embodiments, the technology provides a composition comprising a compound shown above and/or described herein. In some embodiments, the technology provides a pharmaceutical composition comprising a compound shown above and/or described herein. In some embodiments, the pharmaceutical composition is formulated for oral administration.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A to FIG. 1C show C9orf72 HRE-associated DAXX condensates in ALS patient cells. FIG. 1A shows co-localization of the C9HRE DNA locus with one of the DAXX condensates. Representative images show DNA FISH analysis of the HRE locus using an Alexa Fluor 488-labeled (C4G2)₄ ssDNA probe (SEQ ID NO: 15), with co-immunostaining for DAXX, for C9-ALS patient B lymphocytes harboring an approximately 2,600 G4C2 repeat and control cells without the expanded repeat. A focal plane in which the repeat locus and one of the DAXX puncta co-localize (arrowhead) is shown. Scale bar, 10 μm. FIG. 1B shows increased DAXX condensation in C9HRE ALS patient cells. Representative immunostaining images of DAXX in control and C9HRE B lymphocytes are shown; 6-22 fields containing 24 to 87 cells of each line were examined.

Each dot on the graphs represents the average number of DAXX puncta in a field, and the average number of DAXX puncta in each cell line was used for statistical analysis (n=3-5 independent lines). Scale bar, 10 μm. FIG. 1C shows increased DAXX condensation in C9HRE ALS patient-derived motor neurons. Representative immunostaining images of DAXX in control and C9HRE motor neurons are shown. Ten fields containing 72 to 117 neurons from two independent slides for each cell line were examined (n=3 independent pairs of iMN lines). Scale bar, 3 m. See also FIG. 8A to FIG. 8G.

FIG. 2A to FIG. 2I show that liquid-liquid phase separation of DAXX reorganizes chromatin topology and spatial transcription. FIG. 2A and FIG. 2B are time-lapse images of the liquid-liquid phase separation of nuclear Opto-DAXX. Upon exposure to blue-light illumination, DAXX-mCherry-CRY2 is translocated to the nucleus and forms discrete yet fusible condensates, while the mCherry-CRY2 control remains mostly diffusely detected in the cytoplasm in HEK293 cells. Scale bars, 10 μm (FIG. 2A) and 2 μm (FIG. 2B). FIG. 2C shows ATAC-PALM visualization of accessible chromatin sites. FIG. 2D are 2D ATAC-PALM images showing the accessible chromatins spatially restructured by the liquid-liquid phase separation of Opto-DAXX, activated by blue-light illumination. Scale bar, 5 μm. FIG. 2E shows quantification of the co-localization of Opto-DAXX condensates and accessible chromatin sites; 167-234 condensates were statistically analyzed in each group. FIG. 2F shows representative images and quantification of signals for H3K9me3 (tri-methylation at the 9th lysine residue of the histone H3 protein) and nascent RNA at Opto-DAXX droplets after a 6-h illumination with blue light. Scale bar, 5 μm. FIG. 2G shows genome interactions profiled by IIiChIP among regulatory regions including promoters (P), enhancers (E), and gene bodies (GB), with or without liquid-liquid phase separation of Opto-DAXX as a result of blue-light illumination for 10 min. FIG. 2H shows quantification of global chromatin accessibility, measured by ATAC-seq in six C9HRE and four control iMN lines (n=6-10 biological replicates; different colors represent samples from independent iMN lines). FIG. 2I shows heatmaps of peak coverage 1 kbp upstream and downstream of all TSSs for the ATAC-seq data from the C9HRE and control iMN lines in (H). See also FIG. 9A to 9F.

FIG. 3A to FIG. 3P show that increases in nuclear DAXX and its condensation are associated with epigenetic dysregulation in C9HRE patient cells. FIG. 3A to FIG. 3E show immunoblotting of DAXX and related epigenetic regulators (ATRX, suv39h1, HDAC1) in control and C9HRE iMNs (n=3 independent pairs of iMN lines; each dot represents a biological replicate). FIG. 3F and FIG. 3G show immunoblotting of DAXX and suv39h1 in the cervical spinal cords from C9HRE patients and controls (n=6-8). FIG. 3H shows ATRX condensates in control and C9HRE iMNs, visualized by immunostaining (n=3 independent pairs of iMN lines; each dot represents the percentage of puncta area in a ROI). FIG. 3I shows immunostaining of nuclear PML puncta in control and C9HRE iMNs (n=3 independent pairs of iMN lines; each dot represents the average number of puncta per nucleus in a field of view). FIG. 3J shows association of HDAC1 with DAXX in the nuclei of control and C9HRE iMNs, as shown by co-immunostaining (n=3 independent pairs of iMN lines; each dot in the graph represents the Pearson's correlation coefficient in a nucleus). FIG. 3K and FIG. 3M show immunoblotting of H3K9me3 and H3K27ac (acetylation of the lysine residue at position 27 of the histone H3 protein) normalized against total Histone 3 in control and C9HRE iMNs (n=3 independent pairs of iMN lines; each dot represents a biological replicate). FIG. 3N to FIG. 3P show that knockdown of DAXX reduces the level of H3K9me3 but increases that of H3K27ac in the C9HRE iMNs (n=6 biological replicates; different shapes of dots represent independent iMN lines). The level of total Histone 3 was used for normalization. See also FIG. 10A to FIG. 10H.

FIG. 4A to FIG. 4J show that DAXX mediates HRE-associated chromatin abnormalities and transcriptional repression at the C9V2 promoter in C9HRE iMNs. FIG. 4A and FIG. 4B show chromatin accessibility at the C9V2 promoter locus, as indicated by peak coverage and heatmaps (FIG. 4A) and the quantitation of the ATAC signals (FIG. 413) in control and C9HRE iMNs (n=6-10 biological replicates; different colors represent independent iMN lines). FIG. 4C and FIG. 4D show ChIP-qPCR analysis of the occupancies of endogenous II3K9me3, II3K27ac, and RNA Pol II at the C9V2 promoter region in control and C9HRE iMNs (n=3 independent pairs of iMN lines). FIG. 4E to FIG. 4G show ChIP-qPCR analysis of the occupancies of H3K9me3, H3K27ac, and RNA Pol II at the C9V2 promoter region in C9HRE iM4Ns upon the knockdown of DAXX (n=6 biological replicates; different shapes of dots represent independent iMN lines). FIG. 4H to FIG. 4J show immunoblotting of C9orf72 protein (FIG. 4H and FIG. 4I) and RT-qPCR analysis of the C9V2 mRNA (FIG. 4J) in C9HRE iMNs upon the knockdown of DAXX (n=6 biological replicates; different shapes of dots represent independent iMN lines). See also FIG. 11A to 11G and FIG. 12A to FIG. 12G.

FIG. 5A to FIG. 5F show that stress-dependent induction of C9orf72 is impaired in C9HRE ALS patient cells. FIG. 5A to FIG. 5C show immunoblotting of C9orf72 protein (FIG. 5A and FIG. 5B) and RT-qPCR analysis of the C9V2 mRNA (FIG. 5C) in control and C9HRE iMNs treated with 5 sg/ml tunicamycin (TM) or DMSO for 24 h (n=3 independent iMN lines; each dot represents a biological replicate). FIG. 5D to FIG. 5F show immunoblotting of C9orf72 protein (FIG. 5D and FIG. 5E) and RT-qPCR analysis of the C9V2 mRNA (FIG. 5F) in control and C9HRE B lymphocytes treated with 1 μg/ml TM or DMSO for 24 h (n=3-5 independent cell lines; each dot represents a biological replicate). See also FIG. 13A to FIG. 13M.

FIG. 6A to FIG. 6F show that DAXX phase separation mediates HRE-associated C9orf72 suppression. FIG. 6A and FIG. 6B show RT-qPCR analysis of stress-induced expression of the C9V2 mRNA in C9HRE iMNs and human B lymphocytes upon knockdown of DAXX by shRNAs. The cells were treated with 5 μg/ml tunicamycin (TM) or DMSO for 24 h (n=6 biological replicates; different shapes of dots represent independent iMN lines). FIG. 6C shows fold changes in the C9V2 mRNA levels upon DAXX-Flag overexpression in human RPE1 cells. An empty vector and a GUS-Flag overexpression served as controls (n=3 biological replicates). FIG. 6D shows fold changes in the C9V2 mRNA levels in human RPE1 cells expressing a control shRNA or shRNAs targeting DAXX (n=3 biological replicates). FIG. 6E shows TAD analysis of DAXX HiChIP for chromosome 9 and the C9orf72 locus in HEK293 cells expressing Opto-DAXX with or without blue-light illumination. Resolution is set to 1 Mbp or 50 kbp for the whole chromosome 9 or the C9orf72 locus, respectively. FIG. 6F shows virtual chromatin contact profiles derived from the DAXX HiChIP analysis of the C9orf72 locus, with references to the ChIP-seq data for DAXX, CTCF, and H3K27ac in the region. See also FIG. 9A to FIG. 9F.

FIG. 7A to FIG. 7E show that DAXX regulates the susceptibility of C9orf72 HRE iMNs to proteotoxic stress. FIG. 7A and FIG. 7B show that knockdown of DAXX increases the survival of C9HRE iMNs under the stress of tunicamycin (TM) treatment (5 g/ml). Neuronal survival was measured by calcium staining at the indicated time points (n=6 biological replicates; different colors represent independent iMN lines). FIG. 7C and FIG. 7D show results from experiments in which COHRE iMNs were stressed with TM (5 μg/ml) and simultaneously treated with Na-Phen (10 M), 5-aza-2 (2 μM), or DMSO. Neuronal survival was measured by calcium staining at the indicated time points (n=6 biological replicates; different colors represent independent iMN lines). FIG. 7E shows pathological cascades of chromatin architectural and epigenetic abnormalities initiated by C9orf72 HRE-dependent DAXX condensation in patient cells. Abnormal accumulation of nuclear DAXX condensates, as a result of the expanded hexanucleotide repeats, drives genome-wide chromatin structural changes and epigenetic dysregulation in C9orf72 HRE ALS/FTD patient cells. At the C9orf72 locus, the major C9orf72 transcript is stress-inducible at the transcriptional level, but the HRE mutation blocks the stress-dependent induction of C9orf72 in patient cells through DAXX-mediated chromatin remodeling. The loss of transcriptional plasticity of the C9orf72 gene compromises the survival fitness of neurons under stress and may therefore contribute to the neurodegeneration in ALS/FTD and relevant diseases.

FIG. 8A to FIG. 8G shows identification of DAXX as a G4C2 DNA repeat-binding protein (See also FIG. 1A to FIG. 1C). FIG. 8A is a schematic illustration of stable isotope labeling with amino acids (SILAC)-based quantitative proteomic analysis to identify G4C2 DNA repeat-binding proteins. FIG. 8B shows that proteins functioning in transcription are over-represented in the identified G4C2 DNA repeat-binding proteins. FIG. 8C shows immunoblotting of DAXX in the precipitates pulled down by a random or (G4C2)₆ dsDNA biotin-labeled probe (SEQ ID NO: 21, 22) from nuclear fractions (n=3 biological replicates). FIG. 8D shows Coomassie Brilliant Blue staining of DAXX protein purified from E. coli. FIG. 8E and FIG. 8F show EMSA analysis of the interaction between DAXX protein and the (G4C2)₁₀ dsDNA probe (n=3 independent experiments) produced by annealing ssDNA sequences provided by SEQ ID NO: 17 and SEQ ID NO: 18. FIG. 8G shows representative images of C9-ALS patient B lymphocytes harboring an approximately 1,100 G4C2 repeat and of control cell without the expanded repeat, as revealed by DNA FISH analysis of the HRE locus using an Alexa Fluor 488-labeled (C4G2)₄ (SEQ ID NO: 15) ssDNA probe, with co-immunostaining for DAXX. A focal plane where the repeat locus and one of the DAXX puncta co-localize (arrowhead) is shown. Scale bar, 10 m.

FIG. 9A to FIG. 9F show that liquid-liquid phase separation of DAXX in the nucleus drives remodeling of chromatin structures (see FIG. 2A to 2I and FIG. GA to FIG. GF). FIG. 9A shows analysis of DAXX protein using Predictor of Natural Disordered Regains (PONDR) software. FIG. 9B and FIG. 9C are time-lapse images of the dynamic changes in chromatin structures during the phase separation of Opto-DAXX upon illumination with blue light. Scale bar, 10 μm. FIG. 9D shows that Opto-DAXX droplets are not associated with RNA polymerase II. HEK293 cells expressing Opto-DAXX were exposed to blue-light illumination for 6 h, and the co-localization of Opto-DAXX with RNA polymerase II was analyzed by fluorescence imaging and co-immunostaining assays. Each column represents data from a single cell (n=22). Scale bar, 4 μm. FIG. 9E shows ChIP-qPCR analysis of endogenous DAXX occupancy at the C9V2 promoter region in human RPE1 cells (n=3 biological replicates). FIG. 9F is a schematic model of heterochromatin formation and transcription insulation driven by the liquid-liquid phase separation of DAXX.

FIG. 10A to FIG. 10H show that DAXX and associated histone chaperone complexes are dysregulated in C9orf72 HRE patient cells (refer to FIG. 3A to FIG. 3P). FIG. 10A and FIG. 10B show immunoblotting of DAXX in multiple control and a number of C9HRE patient B lymphocyte lines. Actin, PARP, and GAPDH were used as normalization controls for whole-cell lysates and nuclear and cytoplasmic fractions, respectively (n=3-5 independent cell lines; each dot represents a biological replicate). FIG. 10C to FIG. 10E show co-immunostaining of DAXX and ATRX in control and C9HRE B lymphocyte lines (FIG. 10C). Quantification of ATRX puncta (FIG. 10D) and colocalization analysis of DAXX with ATRX (FIG. 10E) in control and C9HRE B lymphocyte lines are shown (n=3-5 independent cell lines); 6 to 22 fields containing 24 to 87 cells were examined for each cell line. Each dot represents the average number of puncta per nucleus in a field; scale bar, 10 m. FIG. 10F to FIG. 10H show co-immunostaining of DAXX and HDAC1 in control and C9HRE B lymphocyte lines (FIG. 10F). Quantification of HDAC1 puncta (FIG. 10G) and co-localization analysis of DAXX with HDAC1 (FIG. 10H) in control and C9HRE B lymphocyte lines are shown (n=3-5 independent cell lines); 9-13 fields containing 106-239 cells were examined for each cell line. Each dot represents the average number of puncta per nucleus in a field; scale bar, 10 μm.

FIG. 11A to FIG. 11G show HRE-associated loss of chromatin accessibility at the C9V2 promoter in C9orf72 ALS patient cells (refer to FIG. 4A to FIG. 4J). FIG. 11A shows schematics of C9orf72 transcript variants and positions of their specific primer sets. FIG. 11B and FIG. 11C show relative levels of C9orf72 V1-3 transcript variants in human RPE1 cells and human iPSCs (n=3 biological replicates). FIG. 11D shows promoter analysis of the 5-kb region upstream of the TSS for C9V2 using the Eukaryotic Promoter Database. The GC boxes with a p-value less than 1×10⁻³ are shown. FIG. 11E to FIG. 11G show experimental design and tests for the promoter activity of the C9orf72 intron 1b region. An EGFP vector without a promoter was used as the plasmid backbone, and the promoter activity of intron la or intron 1b sequences were tested (FIG. 11E). The EGFP protein or mRNA level was detected by fluorescence intensity (FIG. 11E), immunoblotting (FIG. 11F), or RT-qPCR analysis (FIG. 11G) (n=3 biological replicates). Scale bar, 50 μm.

FIG. 12A to FIG. 12G show that DAXX knockdown increases C9orf72 V2 transcript levels in C9orf72 HRE patient cells (refer to FIG. 4A to FIG. 4J). FIG. 12A shows chromatin accessibility at the C9V2 promoter locus, as indicated by peak coverage and heatmaps in control and C9HRE B lymphocytes. FIG. 12B shows ChIP-qPCR analysis of the C9V2 promoter region pulled down by an antibody against RNA polymerase II in three control and five C9HRE patient B lymphocyte lines (n=3-5 independent cell lines; each dot represents a biological replicate). FIG. 12C shows ChIP-qPCR analysis showing that DAXX knockdown via shRNAs increased the occupancy of RNA polymerase II at the C9V2 promoter region in C9HRE patient B lymphocytes (n=6 biological replicates; different shapes of dots represent independent cell lines). FIG. 12D to FIG. 12G show immunoblotting and RT-qPCR analysis of C9orf72 protein and C9V2 mRNA levels, respectively, in C9HRE iPSCs (FIG. 12D and FIG. 12E) and B lymphocytes (FIG. 12F and FIG. 12G) with or without DAXX knockdown (n=6 biological replicates; different shapes of dots represent independent cell lines).

FIG. 13A to FIG. 13M show stress-induced transcription of C9orf72 in various cell lines and loss of this transcriptional regulation in C9-ALS patient cells (refer to FIG. 5A to FIG. 5F). FIG. 13A to FIG. 13E show stress-induced changes in the levels of C9orf72 pre-mRNA after treatment with tunicamycin (TM, 1 μg/ml, 24 h) (FIG. 13A) or thapsigargin (TG, 40 nM, 24 h) (FIG. 13D), C9V2 mRNA (FIG. 13E), or the full-length C9orf72 protein (FIG. 13B and FIG. 13C) in control or C9-ALS patient B lymphocytes (n=3-5 independent lines; each cell line was repeated three times). FIG. 13F and FIG. 13G show stress-dependent induction of C9V2 mRNA, as measured by RT-qPCR, in HEK293 cells (FIG. 13F) and human RPE1 cells (FIG. 13G) after treatment with increasing concentrations of TM or TG for 24 h (n=3 independent experiments). FIG. 13H shows immunoblot analysis of C9orf72 protein levels in human RPE1 cells after treatment with TM (5 μg/ml) or TG (30 nM) for 24 h (n=3 independent experiments). FIG. 13I shows ChIP-qPCR analysis of the C9V2 promoter region pulled down by antibodies against H3K27ac or H3K9me3 in human RPE1 cells after treatment with TM (5 μg/ml), TG (30 nM), or DMSO for 24 h (n=3 independent experiments). FIG. 13J shows RT-qPCR analysis of C9V2 mRNA levels in human RPE1 cells after treatment with Na-Phen (10 μM), 5-aza-2 (2 μM), or DMSO for 24 h (n=3 independent experiments). FIG. 13K to FIG. 13M show immunoblot analysis of C9orf72 protein levels (FIG. 13K) and RT-qPCR analysis of the C9orf72 mRNA variants (FIG. 13L and FIG. 13M) in human RPE1 cells with or without the expression of PR82 poly-dipeptides (n=3 independent experiments).

FIG. 14A to FIG. 14C show that knockdown of DAXX does not change the levels of C9V1 or C9V3 transcripts. FIG. 14A shows that the levels of the C9V1 and C9V3 mRNAs were unchanged by the knockdown of DAXX in C9HRE iMNs (n=6 biological replicates; different shapes of dots represent independent iMN lines). FIG. 14B and FIG. 14C show FISH analysis of RNA foci containing G4C2 repeat RNAs in C9HRE iMNs after knockdown of DAXX (6-9 fields containing 68-110 neurons were analyzed in each group; different shapes of dots represent independent iMN lines, and each dot represents the average number of the foci per nucleus in a field of view).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to treatment of diseases caused by hexanucleotide repeat expansion in the C9orf72 gene and particularly, but not exclusively, to methods of treating disease by decreasing the activity of death domain-associated protein (DAXX) and/or normalizing histone modifications and chromatin structure throughout the human genome.

The C9orf72 hexanucleotide repeat expansion (HRE) is the most frequent genetic cause of amyotrophic lateral sclerosis (ALS) and frontotemporal dementia (FTD). During the development of the technology described herein, experiments were conducted that identified the molecular cascades initiated by the HRE DNA and mediated by death domain-associated protein (DAXX). DAXX preferentially binds to HRE DNA and consequently becomes enriched in the nuclei of HRE-harboring cells. Accumulation of DAXX promotes its phase separation (e.g., condensation) and drives chromatin remodeling and epigenetic changes such as histone hypermethylation and hypoacetylation. A stress-dependent induction of C9orf72 is blocked by upregulation of DAXX by chromatin remodeling and epigenetic modifications of the promoter of the major C9orf72 transcript. Downregulation of DAXX or rebalancing the epigenetic modifications mitigates the stress-induced sensitivity of C9orf72 patient-derived motor neurons. Accordingly, data produced during these experiments identified a C9orf72 HRE DNA-dependent regulatory mechanism for both local and genomic architectural changes in the relevant diseases.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., an analyte). For example, when an analyte is said to be “present” in a test sample, it means the level or amount of this analyte is above a pre-determined threshold; conversely, when an analyte is said to be “absent” in a test sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In some embodiments, the subject has a neurodegenerative disorder (e.g., amyotrophic lateral sclerosis, frontotemporal dementia).

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can comprise, but are not limited to, test tubes and cell culture. The term “in vivo” refers to the natural environment (e.g., an animal or a cell) and to processes or reaction that occur within a natural environment.

As used herein, the terms “test compound” and “candidate compound” refer to any chemical entity, pharmaceutical, drug, and the like that is a candidate for use to treat or prevent a disease, illness, sickness, or disorder of bodily function (e.g., a neurodegenerative disorder (e.g., amyotrophic lateral sclerosis, frontotemporal dementia)). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo or ex vivo.

As used herein, the term “knockdown” means that expression of one or more genes (e.g., in an organism) is reduced, typically significantly, with respect to a functional gene, e.g., reduced to a therapeutically effective degree. Gene knockdown also includes complete gene silencing. As used herein, “gene silencing” means that expression of a gene is essentially completely prevented. Knockdown and gene silencing may occur either at the transcriptional stage or the translational stage. Accordingly, in some embodiments, “knockdown” results in a decrease in the expression level of a gene product (e.g., a protein, an mRNA) in a cell. Accordingly, “knockdown” may be used interchangeably with the phrases “reduction of” the levels of a gene product (e.g., protein or mRNA), “reduction in the expression level of” a gene product (e.g., protein or mRNA), or any variation of these phrases.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

As used herein, the term “HRE” is an abbreviation used to refer to a hexanucleotide repeat expansion and the term “C9HRE” is an abbreviation used to refer to a C9orf72 hexanucleotide repeat expansion.

### Chromatin Structure and Disease

The expansion of the repeat DNA sequences has been linked to an increasing number of human neurological disorders. However, the molecular functions of the C9orf72 hexanucleotide repeat, especially at the DNA level, remain largely undefined.

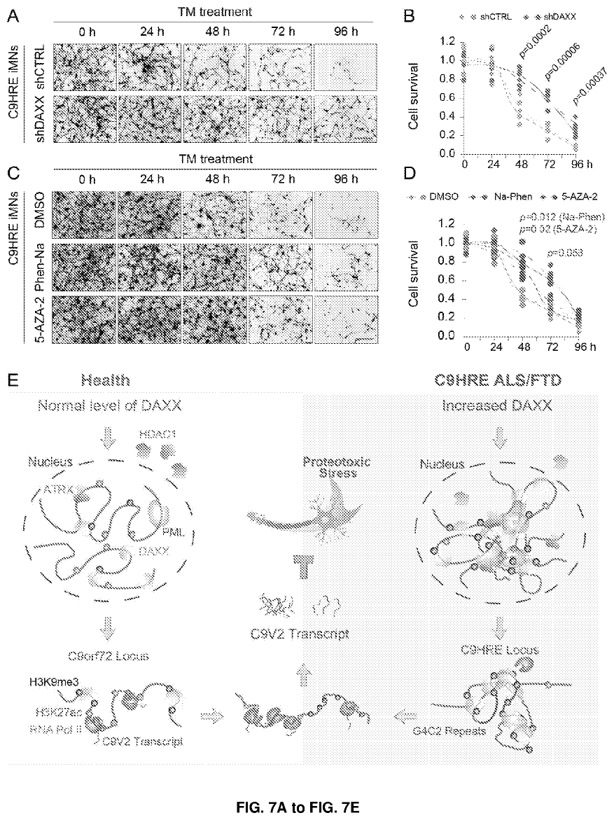

During the development of embodiments described herein, DAXX was identified as a key DNA-binding protein that recognizes the C9orf72 HRE DNA and undergoes HRE-dependent phase separation and protein condensation, leading to global chromatin remodeling and epigenetic dysregulation in patient cells (FIG. 7E). Chromatin conformations and epigenetic modifications cause changes in genomic physical structure and gene expression and thus underlie transcriptional dysregulation in many neurodegenerative diseases (Berson et al., 2018). The eukaryotic genome is organized hierarchically and spatially, and chromosomes can fold into units of tens to hundreds of kilobases that are known as topologically associating domains (TAD) (Dixon et al., 2012), a conserved feature of genome organization that enables preferential local or long-range interactions within the domains. The dynamics of chromatin structure are closely related to epigenetic regulations such as modifications of DNA and histones. Phase separation of chromatin-interacting proteins such as chromatin remodeling complexes and transcription mediators can drive the formation of chromatin compartmentation and long-range interactions (Fasciani et al., 2020; Huo et al., 2020; Shin et al., 2018), and aberrant chromatin remodeling and epigenetic modifications have been implicated in the etiology of ALS (Paez-Colasante et al., 2015; Sun et al., 2018). In C9orf72 HRE-linked ALS and FTD, hypermethylated DNA and altered histone modifications have been observed in the repeat DNA and nearby sequences (Belzil et al., 2013; Liu et al., 2014; Xi et al., 2013).

The nuclear accumulation of DAXX in HRE-harboring cells suppresses a stress-dependent induction of the expression of C9orf72 through the regulation of chromatin structures and epigenetic modifications. Data collected during experiments described herein have revealed the mechanisms through which C9orf72 HRE DNA and its key protein partner reshape genomic architectures and initiate pathogenic cascades that lead to compromised cell fitness.

DAXX was identified as a C9HRE DNA-binding protein that recognizes the (G4C2)ₙ hexanucleotide repeat DNA and undergoes a series of changes in an HRE-dependent manner. Through its interaction with the HRE DNA and the resulting increase in its nuclear concentration, DAXX undergoes enhanced liquid-liquid phase separation and molecular condensation. Likely existing in an equilibrium between its different phases, DAXX accumulates and condenses at the C9orf72 HRE site and throughout the nuclei in HRE-containing patient cells. By modeling the phase separation of DAXX using an optogenetic system, observations indicated that the phase separation of DAXX drives chromatin remodeling, epigenetic changes, and gene expression regulation in the whole genome. DAXX binds with regulatory sequences, including promoters and enhancers, and through phase separation pulls them together in remodeled chromatin structures. With the recruitment of histone modification proteins such as ATRX, SUV39H1, and HDAC1, DAXX promotes an increase in the transcription suppression marker H3K9me3 and a decrease in the transcription activation marker I13K27ac in gene regulatory regions. Consequently, the occupancy of RNA polymerases is decreased, and transcription is suspended. These results provide a mechanistic basis for the alterations in genomic topology in patient cells harboring the C9orI72 HRE mutations, consistent with the architectural changes in chromatin observed in C9orf72-ALS and C9orf72-FTD patient tissues (Appleby-Mallinder et al., 2021: Sun et al., 2018).

The HRE-dependent nuclear accumulation of DAXX has a profound effect on the expression of the C9orf72 gene, not only exemplifying the roles of DAXX in genomic and epigenetic regulation but also providing insights into the disease mechanisms related to the expression of C9orf72. One of the surprising findings indicated by the results of experiments conducted during development of the technology was the stress-inducible expression of C9orf72, which could be important for the maintenance of a state of fitness in cells, especially under disease-associated stress conditions. DAXX plays a critical role in the stress-dependent induction of C9orf72 by recognizing the predominant V2 transcript promoter, which is GC-rich like the hexanucleotide repeat. DAXX negatively regulates the expression of C9orf72 by modulating the V2 promoter epigenetic modifications, including H3K9me3 and H3K27ac. HiChIP analysis revealed that the phase separation of DAXX drives the changes in the 3D genomic interactions of the C9orf72 V2 promoter and renders the promoter inactive in reorganized chromatin structures. In patient cells, the stress-dependent induction of C9orf72 was blocked, with elevated levels of DAXX suppressing the activity of the C9orf72 V2 promoter on both the HRE mutant and wild-type alleles. Since the heterozygous HRE mutation induced an increase in nuclear DAXX, thereby leading to development of the phenotype from both alleles, these results provide a dominant-negative mechanism that may underlie the contributions of the haploinsufficiency of C9orf72 to the diseases. Notably, DAXX did not influence the levels of either the V1 or V₃ transcript, both of which start upstream of the C9orf72 V2 promoter region, but it specifically regulated the expression of the predominant V2 transcript. Consistently, knockdown of DAXX did not change the levels of HRE-containing RNA foci or the DPR products, which are generated from the V1 or V3 transcript, but it significantly enhanced the resistance of patient-derived iMNs to stress-induced toxicity, demonstrating the protective effects of DAXX-dependent stress-inducible expression of the C9orf72 V2 transcript.

In addition to a role for DAXX in suppressing the C9orf72 transcription as part of the loss-of-function disease mechanisms, the HRE-dependent elevation of DAXX induced genome-wide pathologic changes (e.g., abnormal global chromatin structure and/or epigenetic modification of the genome) that contributed to the gain-of-function disease mechanisms. These HRE-dependent DAXX-mediated pathological changes include a global loss of chromatin accessibility in C9orf72 ALS and FTD patients, as demonstrated by ATAC-seq analysis of patient-derived motor neurons, consistent with the genome-wide DNA hypermethylation observed in the spinal cord neurons of C9orf72 ALS patients (Appleby-Mallinder et al., 2021). Altered epigenetic histone protein modifications have been reported in a mouse model overexpressing a RAN translation poly-dipeptide product (Zhang et al., 2019), and data presented herein indicated a distinct mechanism through which the C9orf72 repeat DNA influences epigenetic histone markers via the HRE-binding protein DAXX. Transcriptional dysregulation associated with changes in chromatin structure and epigenetic modifications are a widespread feature in ALS patients, including those linked to SOD1, FUS, and TDP43 (Barbosa et al., 2010; Berson et al., 2017; Yang et al., 2014). Rebalancing histone hypoacetylation using Na-Phen prolongs the survival in a mutant SOD1 mouse model of ALS (Ryu et al., 2005). Reducing DAXX or rebalancing the histone hypermethylation or hypoacetylation mitigates the sensitivity of C9orf72 ALS patient-derived iMNs to stress. Taken together, data collected during experiments described herein suggest that DAXX-mediated pathogenic cascades, including those causing epigenetic dysregulation, provide new strategies and potential targets for interventions to prevent or treat C9orf72 HRE-associated diseases.

### Compositions and Methods of Treatment

Accordingly, in some embodiments, the technology provided herein relates to compositions for treating neurodegenerative diseases (e.g., ALS, FTD) and related methods of treating a patient with the compositions. In some embodiments, the technology provides a DAXX inhibitor (e.g., a composition that decreases an amount, concentration, and/or activity of DAXX) and methods of treating a patient by administering the DAXX inhibitor. In some embodiments, the technology provides an inhibitor of DAXX activity and methods of treating a patient by administering the inhibitor of DAXX activity. For instance, embodiments provide an anti-DAXX antibody, an aptamer, an antisense molecule, a small RNA, a protein, a small molecule, or other inhibitor of DAXX expression or DAXX activity. In some embodiments, the technology comprises use of de novo peptide targeted therapeutics as described, for example, by Chevalier (2017) Nature 550: 74-79, incorporated by reference herein in its entirety. In some embodiments, the technology relates to a small molecule inhibitor of DAXX, e.g., a small molecule inhibitor of the human DAXX protein. In some embodiments, the technology provides a composition comprising a DAXX inhibitor.

In some embodiments, the technology provides a composition that decreases histone methylation and related methods of treating a subject by administering the composition to the subject. In some embodiments, the technology provides a composition that increases histone acetylation and related methods of treating a subject by administering the composition to the subject. In some embodiments, the technology relates to a histone methyltransferase inhibitor (e.g., to suppress histone methylation) and/or to a histone deacetylase inhibitor (e.g., to enhance histone acetylation). In some embodiments, the technology provides an anti-histone methyltransferase antibody, an aptamer, an antisense molecule, a small RNA, a protein, a small molecule, or other inhibitor of histone methyltransferase. In some embodiments, the technology provides an anti-histone deacetylase antibody, an aptamer, an antisense molecule, a small RNA, a protein, a small molecule, or other inhibitor of histone deacetylase. In some embodiments, the technology provides compositions comprising a DAXX inhibitor, a histone methyltransferase inhibitor, and/or a histone deacetylase inhibitor.

In some embodiments, the technology provides a compound according to structure V or to a composition comprising a compound according to structure V:

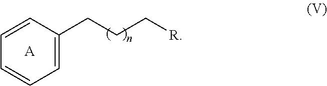

In some embodiments, A is one of

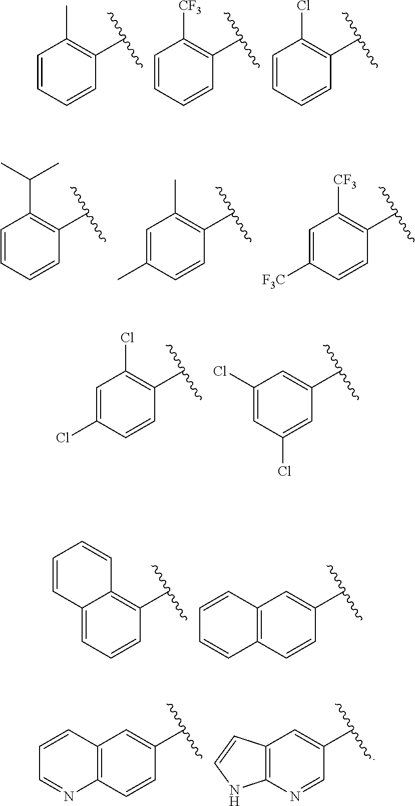

In some embodiments, n=0, 1, 2 3, 4, or 5. In some embodiments, R is CO₂Na or one of

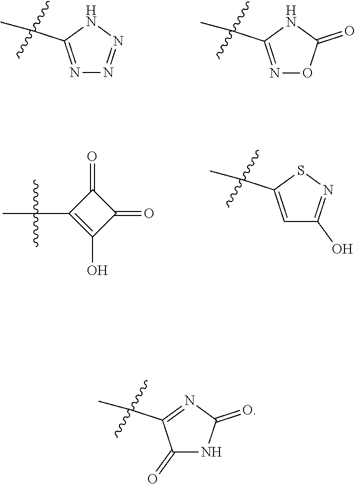 [SMILES: *c1nnn[nH]1 ; *c1noc(=O)[nH]1 ; *c1c(O)c(=O)c1=O ; *c1cc(O)ns1 ; *C1=NC(=O)NC1=O]

In some embodiments, the technology provides a compound according to structure V or to a composition comprising a compound according to structure V:

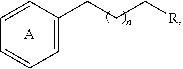

wherein A is one of

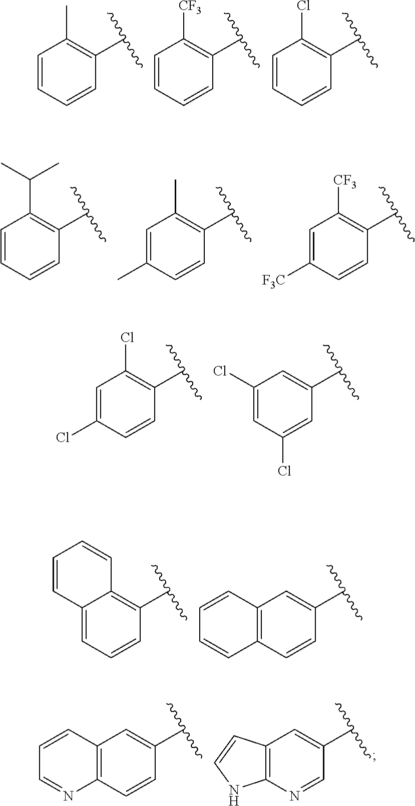

n=0, 1, 2 3, 4, or 5; and R is CO₂Na or one of

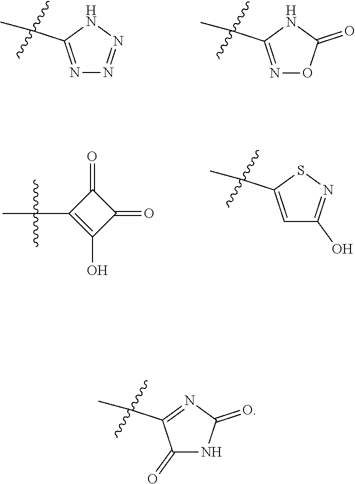 [SMILES: *c1nnn[nH]1 ; *c1noc(=O)[nH]1 ; *c1c(O)c(=O)c1=O ; *c1cc(O)ns1 ; *C1=NC(=O)NC1=O]

In some embodiments, the technology provides a compound according to structure I or to a composition comprising a compound according to structure I:

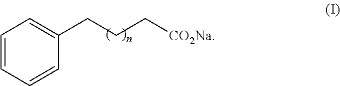

In some embodiments, n=0, 1, 2 3, 4, or 5. Compounds Ia, Ib, Ic, Id, Ie, and If are provided by structure I when n=0, 1, 2, 3, 4, and 5, respectively. In some embodiments, the technology provides a compound according to one of structures Ia, Ib, Ic, Id, Ie, or If or to a composition comprising a compound according to one of structures Ia, Ib, Ic, Id, Ie, or If. In some embodiments, a compound according to structure I is a histone deacetylase inhibitor. In some embodiments, the composition comprises a compound according to the structure (e.g., sodium phenylbutyrate (“Na-Phen”); see, e.g., Warrell (1998) “Therapeutic targeting of transcription in acute promyelocytic leukemia by use of an inhibitor of histone deacetylase” J Natl Cancer Inst 90: 1621-25, incorporated herein by reference):

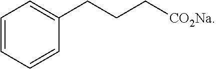

In some embodiments, the technology provides a compound according to structure II or to a composition comprising a compound according to structure 11:

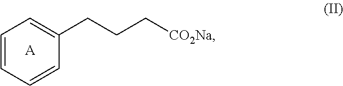

wherein A in structure II is provided by one of the following:

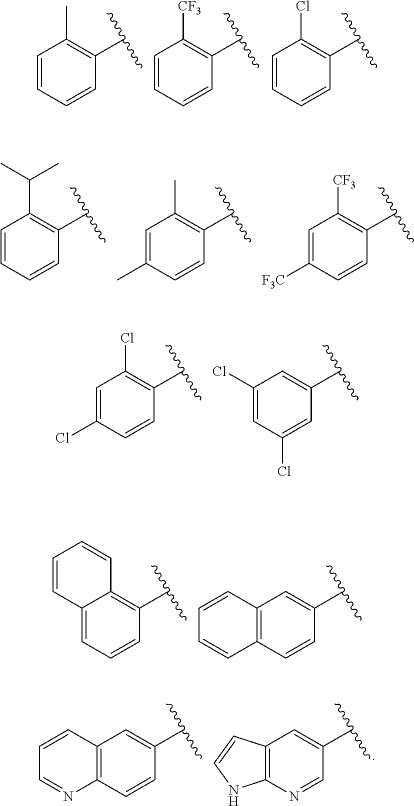

Compounds IIa, IIb, IIc, IId, IIe, IIf, IIg, IIh, IIi, IIj, IIk, and IIl are provided by structure II where A is a substituent as shown above in order from left to right starting at the top row, respectively (i.e., compound IIa is provided by structure II when A is the substituent at the top left above and compound III is provided by structure II when A is the substituent at the bottom right above). In some embodiments, the technology provides a compound having a structure according to one of structures IIa, IIb, IIc, IId, IIe, IIf, IIg, IIh, IIi, IIj, Ilk, or IIl or to a composition comprising a compound having a structure according to one of structures IIa, IIb, IIc, IId, IIe, IIf, IIg, IIh, IIi, IIj, Ilk, or IIl.

In some embodiments, the technology provides a compound according to one of structures IIIa, IIIb, IIIc, IIId, or IIIe (collectively structure III) or to a composition comprising a compound according to one of structures IIIa, IIIb, IIIc, IIId, or IIIe:

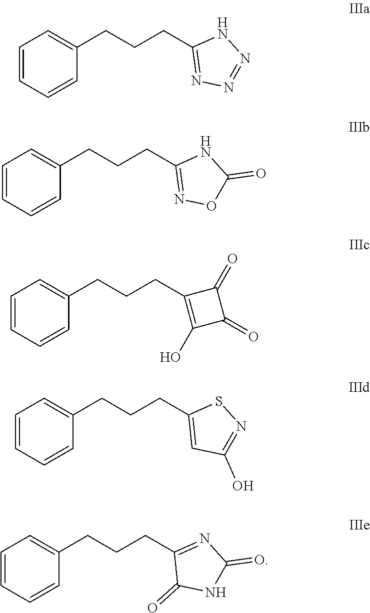

In some embodiments, the technology provides a compound according to one of structures IVa, IVb, IVc, IVd, IVe, IVf, or IVg (collectively structure IV) or to a composition comprising a compound according to structures IVa, IVb, IVc, IVd, IVe, IVf, or IVg:

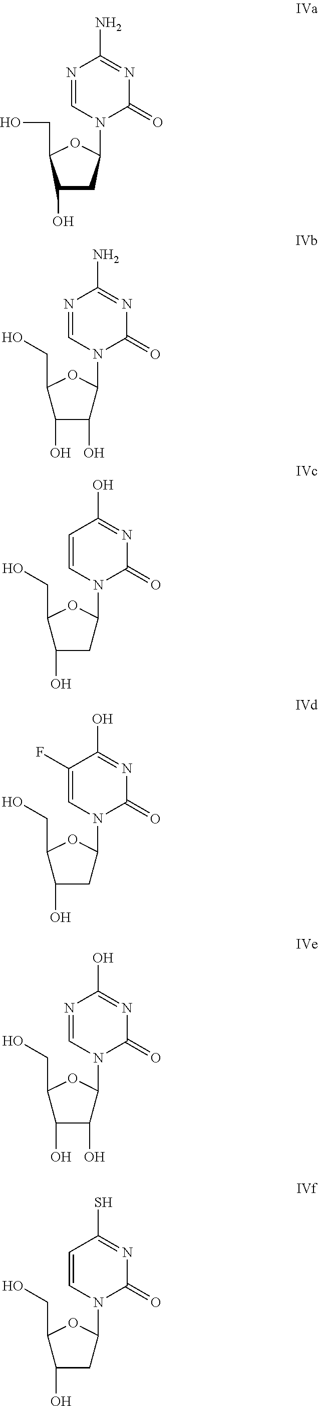

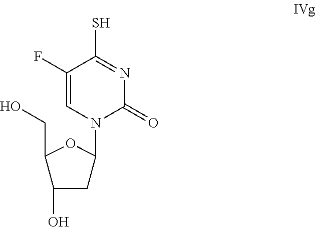 [SMILES: O=c1nc(S)c(F)cn1C1CC(O)C(CO)O1]

In some embodiments, a compound according to structure IV is a histone methyltransferase inhibitor. In some embodiments, the composition comprises a compound according to the structure 1Va (decitabine or 5-aza-2; see, e.g., Nguyen (2002) “Histone H3-lysine 9 methylation is associated with aberrant gene silencing in cancer cells and is rapidly reversed by 5-aza-2′-deoxycytidine” Cancer Res 62: 6456-61, incorporated herein by reference):

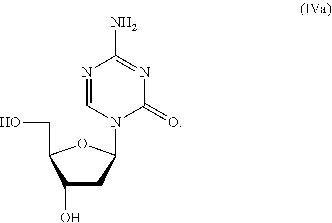

In some embodiments, the technology provides a composition that decreases H3K9me3 occupancy at the promoter of the C9V2 transcript. In some embodiments, the technology provides a composition that increases H3K27ac occupancy at the promoter of the C9V2 transcript.

The technology also relates to methods of treating a subject with a drug appropriate for the subject's malady (e.g., a neurodegenerative disease (e.g., ALS, FTD)). In some embodiments, a method is provided for treating a subject in need of such treatment (e.g., a subject having a neurodegenerative disease (e.g., ALS or FTD)) with an effective amount of a compound described herein (e.g., a DAXX inhibitor or a compound comprising a structure provided by any of structures I, II, II, IV, or V) or a salt thereof.

In some embodiments, a method is provided for treating a subject in need of such treatment (e.g., a subject having a neurodegenerative disease (e.g., ALS or FTD)) with an effective amount of a compound described herein (e.g., a DAXX inhibitor, a compound comprising a structure provided by I or V (e.g., sodium phenylbutyrate or a variant or modified version (hereof (e.g., as provided by Ia, Ib, Ic, Id, Ie, If, IIa, IIb, IIc, IId, IIe, IIf, IIg, IIh, IIi, IIj, IIk, IIl, IIIa, IIIb, IIIc, IIId, or IIIe)), or a compound comprising a structure provided by IV (e.g., decitabine or a variant or modified version thereof (e.g., as provided by IVa, IVb, IVc, IVd, IVe, IVf, or IVg))) or a salt thereof.

The method involves administering to the subject an effective amount of a compound (e.g., a DAXX inhibitor or a compound comprising a structure provided by any of structures I, II, II, IV, or V) or a salt thereof in any one of the pharmaceutical preparations described above, detailed herein, and/or set forth in the claims. The subject can be any subject in need of such treatment. In the foregoing description, the technology is in connection with a compound or salts thereof. Such salts include, but are not limited to, bromide salts, chloride salts, iodide salts, carbonate salts, and sulfate salts. It should be understood, however, that the compound is a member of a class of compounds and the technology is intended to embrace pharmaceutical preparations, methods, and kits containing related derivatives within this class. Another aspect of the technology then embraces the foregoing summary but read in each aspect as if any such derivative is substituted wherever “compound” appears.

In some embodiments, a subject is tested to assess the presence, the absence, or the level of a malady and/or a condition (e.g., ALS, FTD). Such testing is performed, e.g., by detecting, assaying, or measuring a biomarker, a metabolite, a physical symptom, an indication, etc., to determine the risk of or the presence of the malady or condition. In some embodiments, a subject is tested to detect increased DAXX amount or concentration, increased DAXX activity, increased ATRX amount or concentration, abnormal global chromatin structure and/or epigenetic modification of a genome, increased ATRX activity, increased SUV9H1 amount or concentration, increased SUV9H1 activity, increased quantity and/or size of ATRX granules in nuclei, ATRX co-condensed with DAXX, increased PML nuclear bodies (PML-NB), increase in the nuclear localization of HDAC1, HDAC1 co-localized with DAXX, a hexanucleotide (GGGGCC) repeat expansion in the C9orf72 gene, an RNA comprising a hexanucleotide (GGGGCC) repeat expansion, an RNA comprising a G-quadruplex, histone hypermethylation, histone hypomethylation, increased amount or concentration of H3K9me3, decreased amount or concentration of H3K27ac, a DAXX condensate (e.g., in a cell nucleus), aberrant distribution pattern of DAXX throughout nuclei, DAXX accumulation at a G4C2 repeat locus, DAXX accumulation at C9orf72, DAXX granules, increased H3K9me3 occupancy at the C9V2 promoter, decreased H3K27ac occupancy at the C9V2 promoter, decreased RNA polymerase (e.g., RNA polymerase II) occupancy at the V2 promoter, increased toxic peptides (e.g., increased toxic dipeptides (e.g., increased proline-arginine poly-dipeptides)), decreased chromatin accessibility at the C9V2 promoter, reduced amount or concentration of V2 transcript, increased compactness of chromosomes, global loss of chromatin accessibility in C9orf72, genome-wide DNA hypermethylation, altered epigenetic histone protein modifications, transcriptional dysregulation, increased and/or aberrant formation of sub-TADs at the C9orf72 promoter region, and/or changes in chromatin structure and epigenetic modifications.

In some embodiments, a subject is tested for the presence of a hexanucleotide (GGGGCC) repeat expansion in chromosome 9 (e.g., at C9orf72) by obtaining (e.g., sequencing or having sequenced) a nucleotide sequence from a sample obtained from the patient. In some embodiments, a subject is tested for the presence of a hexanucleotide (GGGGCC) repeat expansion in chromosome 9 (e.g., at C9orf72) by evaluating a genome nucleotide sequence obtained from a sample obtained from the patient.

In some embodiments, an amount of DAXX protein is measured in a sample obtained from the patient. In some embodiments, an amount of DAXX protein is measured in a sample obtained from the patient using an immunological method. In some embodiments, the amount of DAXX protein is measured using an antibody specific for DAXX protein. In some embodiments, the antibody specific for DAXX protein comprises a fluorescent label.

In some embodiments, a subject is tested for the presence of a hexanucleotide (GGGGCC) repeat expansion in chromosome 9 (e.g., at C9orf72) using a labeled probe comprising a sequence (CCCCGG)ₙ, wherein n=3 to 10 (e.g., 3, 4, 5, 6, 7, 8, 9, or 10). In some embodiments, the labeled probe comprises a fluorescent label. In some embodiments, the labeled probe comprises a fluorescent label at the 3′ end, at the 5′ end, or at one or more internal bases.

In some embodiments, a sample from the subject is tested to measure an amount of pre-mRNA using primers targeted to the junction between exon 2 and intron 3 of C9orf72. In some embodiments, the primers targeted to the junction between exon 2 and intron 3 of C9orf72 comprise SEQ ID NO: 11 and/or SEQ ID NO: 12. In some embodiments, an amount of a V2 messenger RNA is measured. In some embodiments, an amount of a V2 messenger RNA is measured using a nucleotide amplification method and primers comprising SEQ ID NO: 2 and/or SEQ ID NO: 4. In some embodiments, C9orf72 messenger RNA is detected and/or measured using a labeled probe, e.g., comprising a sequence (CCCCGG)ₙ, wherein n=3 to 10 (e.g., 3, 4, 5, 6, 7, 8, 9, or 10).

In some embodiments, the subject is treated with a compound described herein (e.g., a DAXX inhibitor or a compound comprising a structure provided by any of structures I, II, II, IV, or V) based on the outcome of the test. In some embodiments, a subject is treated, a sample is obtained, and the level of detectable agent is measured, and then the subject is treated again based on the level of detectable agent that was measured. In some embodiments, a subject is treated, a sample is obtained, and the level of detectable agent is measured, the subject is treated again based on the level of detectable agent that was measured, and then another sample is obtained, and the level of detectable agent is measured. In some embodiments, other tests (e.g., not based on measuring the level of detectable agent) are also used at various stages, e.g., before the initial treatment as a guide for the initial dose. In some embodiments, a subsequent treatment is adjusted based on a test result, e.g., the dosage amount, dosage schedule, identity of the drug, etc. is changed. In some embodiments, a patient is tested, treated, and then tested again to monitor the response to therapy and/or change the therapy. In some embodiments, cycles of testing and treatment may occur without limitation to the pattern of testing and treating, the periodicity, or the duration of the interval between each testing and treatment phase. As such, the technology contemplates various combinations of testing and treating without limitation, e.g., test/treat, treat/test, test/treat/test, treat/test/treat, test/treat/test/treat, test/treat/test/treat/test, test/treat/test/test/treat/treat/treat/test, treat/treat/test/treat, test/treat/treat/test/treat/treat, etc.

In some embodiments, methods comprise targeting the DAXX coding sequence and/or a DAXX messenger RNA by a CRISPR technology. In some embodiments, the DAXX coding sequence is targeted by a ribonucleoprotein (RNP) comprising a CRISPR protein (e.g., Cas9 or a protein having the same or similar activity as Cas9) and a guide RNA. In some embodiments, a DAXX messenger RNA is targeted by a ribonucleoprotein (RNP) comprising a CRISPR protein (e.g., Cas13 or a protein having activity that is the same or similar to Cas13) and a guide RNA. In some embodiments, the CRISPR protein is Cas9 or a similar RNA-guided endonuclease having the same on similar activity. Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and destroys foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference); Jinek et al. (2012) “A Programmable Dual-RNA-Guided DNA Endonuclease in Adaptive Bacterial Immunity” Science 337:816-821; and Lee et al. (2016) “The Neisseria meningitidis CRISPR-Cas9 System Enables Specific Genome Editing in Mammalian Cells” Molecular Therapy 24: 645 (2016), each of which is incorporated herein by reference).

In some embodiments, the Cas13 protein is Cas13d, Cas13Rx, another Cas13 protein described herein or known in the art, or a protein having an activity similar to a Cas13 protein such as, e.g., Cas13d, Cas13R, Cas13Rx, or another Cas13 protein described herein or known in the art. See, e.g., Per6ulija (2021) “Functional Features and Current Applications of the RNA-Targeting Type VI CRISPR-Cas Systems” Adv. Sci. 8: 2004685, incorporated herein by reference. Some non-naturally occurring, engineered CRISPR systems and methods for targeted modification of a nucleic acids and methods for computational identification of CRISPR proteins from nucleotide sequences, such as Cas13 proteins, are described, e.g., in U.S. Pat. App. Pub. No. 2019/0002875, incorporated herein by reference. See, e.g., Abudayyeh (2016) “C2c2 is a single-component programmable RNA-guided RNA-targeting CRISPR effector” Science 353(6299):aaf5573; Konermann (2018) “Transcriptome Engineering with RNA-Targeting Type VI-D CRISPR Effectors” Cell 173: 665-676 e614; Yang (2019) “Dynamic Imaging of RNA in Living Cells by CRISPR-Cas13 Systems” Mol Cell 76: 981-997 e987: Freije (2019) “Programmable Inhibition and Detection of RNA Viruses Using Cas13” Mol Cell 76: 826-837 e811; and Cox (2017) “RNA editing with CRISPR-Cas13” Science 358: 1019-1027, each of which is incorporated herein by reference). See, e.g., Abudayyeh et al. (2017) “RNA targeting with CRISPR-Cas13” Nature 550: 280; Yan (2018) “Cas13d Is a Compact RNA-Targeting Type VI CRISPR Effector Positively Modulated by a WYL-Domain-Containing Accessory Protein” Mol. Cell 70: 327-39, each of which is incorporated herein by reference.

In some embodiments, compositions comprising oligomeric antisense compounds, particularly oligonucleotides, are used to modulate the function of nucleic acid molecules encoding DAXX, ultimately modulating (e.g., decreasing) the amount of DAXX expressed. This is accomplished by providing antisense compounds that specifically hybridize with one or more nucleic acids encoding DAXX. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of DAXX. In the context of the present disclosure, “modulation” means either an increase (stimulation) or a decrease (inhibition) in the expression of a gene. For example, DAXX expression may be inhibited to treat or prevent a neurodegenerative disease such as ALS or FTD.

In some embodiments, nucleic acids are small RNAs, for example, siRNAs. “RNA interference (RNAi)” is the process of sequence-specific, post-transcriptional gene silencing initiated by a small interfering RNA (siRNA). During RNAi, siRNA induces degradation of target mRNA with consequent sequence-specific inhibition of gene expression. An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, encoding DAXX. As used herein, the term “siRNA” is a generic term that encompasses all possible RNAi triggers. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In certain embodiments, the siRNAs are targeted to the nucleotide sequence encoding DAXX. In some embodiments, the length of the duplex of siRNAs is less than 30 base pairs. In some embodiments, the duplex can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the duplex is 19 to 32 base pairs in length. In certain embodiments, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

As used herein, Dicer-substrate RNAs (DsiRNAs) are chemically synthesized asymmetric 25-mer/27-mer duplex RNAs that have increased potency in RNA interference compared to traditional siRNAs. Traditional 21-mer siRNAs are designed to mimic Dicer products and therefore bypass interaction with the enzyme Dicer. Dicer has been shown to be a component of RISC and involved with entry of the siRNA duplex into RISC. Dicer-substrate siRNAs are designed to be optimally processed by Dicer and show increased potency by engaging this natural processing pathway. Using this approach, sustained knockdown has been regularly achieved using sub-nanomolar concentrations. (see, e.g., U.S. Pat. No. 8,084,599; Kim (2005) Nature Biotechnology 23: 222; Rose (2005) Nucleic Acids Res., 33: 4140, each of which is incorporated herein by reference).

The transcriptional unit of a “shRNA” comprises sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops comprise sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (approximately 35 nucleotides upstream and approximately 40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In some embodiments, the technology provided herein comprises use of any genetic manipulation for use in modulating (e.g., decreasing) the expression and/or activity of DAXX. Examples of genetic manipulation include, but are not limited to, gene knockout (e.g., removing the DAXX gene from the chromosome using, for example, recombination), expression of antisense constructs with or without inducible promoters, and the like. Delivery of nucleic acid constructs to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and in U.S. Pat. Nos. 6,033,908; 6,019,978; 6,001,557; 5,994,132; 5,994,128; 5,994,106; 5,981,225; 5,885,808; 5,872,154; 5,830,730; and 5,824,544, each of which is herein incorporated by reference in its entirety.

Vectors may be administered to subject in a variety of ways. For example, in some embodiments, vectors are administered into tumors or tissue associated with tumors using direct injection. In other embodiments, administration is via the blood or lymphatic circulation (see, e.g., PCT publication 1999/02685, herein incorporated by reference in its entirety). Exemplary dose levels of adenoviral vector are preferably 10⁸ to 10¹¹ vector particles added to the perfusate.

In some embodiments, the technology provides antibodies that inhibit DAXX. Any suitable antibody (e.g., monoclonal, polyclonal, or synthetic) may be utilized in the therapeutic methods disclosed herein. In some embodiments, the antibodies are humanized antibodies. Methods for humanizing antibodies are well known in the art (see, e.g., U.S. Pat. Nos. 6,180,370; 5,585,089; 6,054,297; and 5,565,332, each of which is herein incorporated by reference).

The present technology is not limited to the use of any particular antibody configuration. In some preferred embodiments, the targeting unit is an antigen binding protein. Preferred antigen binding proteins include, but are not limited to, an immunoglobulin, a Fab, F(ab′)2, Fab′ single chain antibody, Fv, single chain (scFv), mono-specific antibody, bi-specific antibody, tri-specific antibody, multivalent antibody, chimeric antibody, humanized antibody, human antibody, CDR-grafted antibody, shark antibody, an immunoglobulin single variable domain (e.g., a nanobody or a single variable domain antibody), minibody, camelid antibody (e.g., from the Camelidae family), microbody, intrabody (e.g., intracellular antibody), and/or de-fucosylated antibody and/or derivative thereof. Mimetics of binding agents and/or antibodies are also provided.

In some embodiments, scFv polypeptides described herein are fused to Fc regions to generate minibodies. As used herein, the term “fragment crystallizable region (Fc region)” refers to the tail region of an antibody that interacts with cell surface receptors called Fe receptors and some proteins of the complement system. This property allows antibodies to activate the immune system. In IgG, IgA, and IgD antibody isotypes, the Fc region comprises two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site.

In some embodiments, the Fc region is derived from an IgG. In some embodiments, the IgG is human IgG1, although other suitable Fc regions derived from other organisms or antibody frameworks may be utilized.

In some embodiments, scFv polypeptides described herein are fused to chimeric antigen receptors. Chimeric antigen receptors (CARs), (also known as chimeric immunoreceptors, chimeric T cell receptors, artificial T cell receptors or CAR-T) are engineered receptors, which graft an arbitrary specificity onto an immune effector cell (T cell). Typically, these receptors are used to graft the specificity of an antibody (e.g., an scFv described herein) onto a T cell, with transfer of their coding sequence facilitated by retroviral vectors. The receptors are called chimeric because they are composed of parts from different sources.

Further, the present technology also envisages expression vectors comprising nucleic acid sequences encoding any of the above polypeptides or fusion proteins thereof or functional fragments thereof, as well as host cells expressing such expression vectors. Suitable expression systems include constitutive and inducible expression systems in bacteria or yeasts, virus expression systems, such as baculovirus, semliki forest virus and lentiviruses, or transient transfection in insect or mammalian cells. Suitable host cells include E. coli, Lactococcus lactis, Saccharomyces cerevisiae, Schizosaccharomyces pombe, Pichia pastoris, and the like. Suitable animal host cells include HEK 293, COS, S2, CHO, NSO, DT40 and the like. The cloning, expression, and/or purification of the antibodies can be done according to techniques known by the skilled person in the art.

It will be understood that polypeptides described herein may be identified with reference to the nucleotide and/or amino acid sequence corresponding to the variable and/or complementarity determining regions (“CDRs”) thereof.

Also within the scope of the technology are natural or synthetic analogs, mutants, variants, alleles, homologs, and orthologs (herein collectively referred to as “variants”) of the immunoglobulin single variable domains of the technology as defined herein. Thus, according to one embodiment of the technology, the term “immunoglobulin single variable domain” in its broadest sense also covers such variants, in particular variants of the antibodies described herein. Generally, in such variants, one or more amino acid residues may have been replaced, deleted, and/or added compared to the antibodies of the technology as defined herein. Such substitutions, insertions, or deletions may be made in one or more of the framework regions and/or in one or more of the CDRs. Variants, as used herein, are sequences wherein each or any framework region and each or any complementarity determining region shows at least 80% identity, preferably at least 85% identity, more preferably 90% identity, even more preferably 95% identity or, still even more preferably 99% identity with the corresponding region in the reference sequence (e.g., FR1_variant versus FR1_reference, CDR1_variant versus CDR1_reference, FR2_variant versus FR2_reference, CDR2_variant versus CDR2_reference, FR3_variant versus FR3_reference, CDR3_variant versus CDR13_reference, FR4_variant versus FRi4_reference), as can be measured electronically by making use of algorithms such as PILEUP and BLAST. (See, e.g., Higgins & Sharp, CABIOS 5: 151 (1989); Altschul S. F., W. Gish, W. Miller, E. W. Myers, D. J. Lipman. Basic local alignment search tool. J. Mol. Biol. 1990; 215:403-10.) Software for performing BLAST analyses is publicly available through the National Center for Biotechnology Information (on the worldwide web at nebi.nlm.nih.gov). Such variants of immunoglobulin single variable domains may be of particular advantage since they may have improved potency or other desired properties.

A “deletion” is defined herein as a change in either amino acid or nucleotide sequence in which one or more amino acid or nucleotide residues, respectively, are absent as compared to an amino acid sequence or nucleotide sequence of a parental polypeptide or nucleic acid. Within the context of a protein, a deletion can involve deletion of about two, about five, about ten, up to about twenty, up to about thirty or up to about fifty or more amino acids. A protein or a fragment thereof may contain more than one deletion.

An “insertion” or “addition” is a change in an amino acid or nucleotide sequence that has resulted in the addition of one or more amino acid or nucleotide residues, respectively, as compared to an amino acid sequence or nucleotide sequence of a parental protein. “Insertion” generally refers to addition of one or more amino acid residues within an amino acid sequence of a polypeptide, while “addition” can be an insertion or refer to amino acid residues added at an N- or C-terminus, or both termini. Within the context of a protein or a fragment thereof, an insertion or addition is usually of about one, about three, about five, about ten, up to about twenty, up to about thirty, or up to about fifty or more amino acids. A protein or fragment thereof may contain more than one insertion.

A “substitution,” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. By conservative substitutions is intended combinations such as amino acids in the following groups: gly, ala; val, ile, leu, met; asp, glu; asn, gln; ser, thr; lys, arg; cys, met; and phe, tyr, trp.

By means of non-limiting examples, a substitution may, for example, be a conservative substitution (as described herein) and/or an amino acid residue may be replaced by another amino acid residue that naturally occurs at the same position (e.g., for an antibody, in another variable domain). Thus, any one or more substitutions, deletions or insertions, or any combination thereof, that either improve the properties of the antibody of the technology or that at least do not effectively detract from the desired properties or from the balance or combination of desired properties of the antibody of the technology (e.g., to the extent that the antibody is no longer suited for its intended use) are included within the scope of the technology. A skilled person will generally be able to determine and select suitable substitutions, deletions or insertions, or suitable combinations of thereof, based on the disclosure herein and optionally after a limited degree of routine experimentation, which may, for example, involve introducing a limited number of possible substitutions and determining their influence on the properties of the antibodies thus obtained.

Further, depending on the host organism used to express the immunoglobulin single variable domain of the technology, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example, to allow site-specific pegylation.

Examples of modifications, as well as examples of amino acid residues within the immunoglobulin single variable domain, that can be modified (e.g., either on the protein backbone but preferably on a side chain), methods and techniques that can be used to introduce such modifications, and the potential uses and advantages of such modifications will be clear to the skilled person. For example, such a modification may involve the introduction (e.g., by covalent linking or in another suitable manner) of one or more functional groups, residues or moieties into or onto the immunoglobulin single variable domain of the technology, and in particular of one or more functional groups, residues or moieties that confer one or more desired properties or functionalities to the immunoglobulin single variable domain of the technology. Examples of such functional groups and of techniques for introducing them will be clear to the skilled person, and can generally comprise all functional groups and techniques mentioned in the general background art cited hereinabove as well as the functional groups and techniques known for the modification of pharmaceutical proteins, and in particular for the modification of antibodies or antibody fragments (including ScFvs and single domain antibodies), for which reference is, for example, made to Remington's Pharmaceutical Sciences, 16th ed., Mack Publishing Co., Easton, Pa. (1980). Such functional groups may, for example, be linked directly (for example, covalently) to an immunoglobulin single variable domain of the technology, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFvs); reference is made to, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in Int'l Pat. Pub. No. WO04060965. Various reagents for pegylation of proteins are also commercially available, for example, from Nektar Therapeutics, USA. Preferably, site-directed pegylation is used, in particular via a cysteine-residue (see, for example, Yang et al., Protein Engineering, 16, 10, 761-770 (2003). For example, for this purpose, PEG may be attached to a cysteine residue that naturally occurs in an antibody of the technology, an antibody of the technology may be modified so as to suitably introduce one or more cysteine residues for attachment of PEG, or an amino acid sequence comprising one or more cysteine residues for attachment of PEG may be fused to the N- and/or C-terminus of a antibody of the technology, all using techniques of protein engineering known per se to the skilled person. Preferably, for the immunoglobulin single variable domains and proteins of the technology, a PEG is used with a molecular weight of more than 5000, such as more than 10,000 and less than 200,000, such as less than 100,000; for example, in the range of 20,000-80,000.

Another, usually less preferred modification comprises N-linked or O-linked glycosylation, usually as part of co-translational and/or post-translational modification, depending on the host cell used for expressing the immunoglobulin single variable domain or polypeptide of the technology. Another technique for increasing the half-life of an immunoglobulin single variable domain may comprise the engineering into bifunctional constructs or into fusions of immunoglobulin single variable domains with peptides (for example, a peptide against a serum protein such as albumin).

Yet another modification may comprise the introduction of one or more detectable labels or other signal-generating groups or moieties, depending on the intended use of the labeled antibody. Suitable labels and techniques for attaching, using, and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy. Such labeled antibodies and polypeptides of the technology may, for example, be used for in vitro, in vivo or in situ assays (including immunoassays known per se such as ELISA, RIA, EIA and other “sandwich assays,” etc.), as well as in vivo diagnostic and imaging purposes, depending on the choice of the specific label.

As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the antibody of the technology to another protein, polypeptide or chemical compound that is bound to the other half of the binding pair, e.g., through formation of the binding pair. For example, an antibody of the technology may be conjugated to biotin, and linked to another protein, polypeptide, compound or carrier conjugated to avidin or streptavidin. For example, such a conjugated antibody may be used as a reporter, for example, in a diagnostic system where a detectable signal-producing agent is conjugated to avidin or streptavidin. Such binding pairs may, for example, also be used to bind the antibody of the technology to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the antibody of the technology.

In some embodiments, the immunoglobulin single variable domain of the present technology is fused to a detectable label, either directly or through a linker. Preferably, the detectable label is a radio-isotope or radioactive tracer, which is suitable for medical applications, such as in in vivo nuclear imaging. Examples include, without the purpose of being limitative, ^{99m}Tc, ¹²³I, ¹²⁵I, ¹¹¹In, ¹⁸F, ⁶⁴Cu, ⁶⁷Ga, ⁶⁸Ga, and any other radio-isotope which can be used in animals, in particular mouse or human.

In some embodiments, the immunoglobulin single variable domain of the present technology is fused to a moiety selected from the group consisting of a toxin, or to a cytotoxic drug, or to an enzyme capable of converting a prodrug into a cytotoxic drug, or to a radionuclide, or coupled to a cytotoxic cell, either directly or through a linker.

In some embodiments, the present technology provides an antibody-drug conjugate and/or an antibody-enzyme conjugate. In certain embodiments, the antibody drug conjugates are administered to cells expressing DAXX.

As used herein, “linkers” are peptides of 1 to 50 amino acids length and are typically chosen or designed to be unstructured and flexible. These include, but are not limited to, synthetic peptides rich in Gly, Ser, Thr, Gln, Glu or further amino acids that are frequently associated with unstructured regions in natural proteins. (See, e.g., Dosztanyi Z., V. Csizmok, P. Tompa, and I. Simon (2005). IUPred: web server for the prediction of intrinsically unstructured regions of proteins based on estimated energy content. Bioinformatics (Oxford, England), 21(16), 3433-4.)

In some embodiments, the therapeutic polypeptide is an immunoglobulin or fragment thereof. Examples include, but are not limited to, aptamers and immunoglobulins. Immunoglobulins (antibodies) are proteins generated by the immune system to provide a specific molecule capable of complexing with an invading molecule commonly referred to as an antigen. Natural antibodies have two identical antigen-binding sites, both of which are specific to a particular antigen. The antibody molecule recognizes the antigen by complexing its antigen-binding sites with areas of the antigen termed epitopes. The epitopes fit into the conformational architecture of the antigen-binding sites of the antibody, enabling the antibody to bind to the antigen.

The immunoglobulin molecule is composed of two identical heavy and two identical light polypeptide chains, held together by interchain disulfide bonds. Each individual light and heavy chain folds into regions of approximately 110 amino acids, assuming a conserved three-dimensional conformation. The light chain comprises one variable region (termed VL) and one constant region (CL), while the heavy chain comprises one variable region (VH) and three constant regions (CHi, CH2 and CH3). Pairs of regions associate to form discrete structures. In particular, the light and heavy chain variable regions, VL and VH, associate to form an “FV” area that contains the antigen-binding site.

The variable regions of both heavy and light chains show variability in structure and amino acid composition from one antibody molecule to another, whereas the constant regions show little variability. Each antibody recognizes and binds an antigen through the binding site defined by the association of the heavy and light chain, variable regions into an FV area. The light-chain variable region VL and the heavy-chain variable region VH of a particular antibody molecule have specific amino acid sequences that allow the antigen-binding site to assume a conformation that binds to the antigen epitope recognized by that particular antibody.

Within the variable regions are found regions in which the amino acid sequence is extremely variable from one antibody to another. Three of these so-called “hypervariable” regions or “complementarity-determining regions” (CDRs) are found in each of the light and heavy chains. The three CDRs from a light chain and the three CDRs from a corresponding heavy chain form the antigen-binding site.

Cleavage of naturally occurring antibody molecules with the proteolytic enzyme papain generates fragments that retain their antigen-binding site. These fragments, commonly known as Fabs (for Fragment, antigen binding site) are composed of the CL, VL, CH1, and VH regions of the antibody. In the Fab, the light chain and the fragment of the heavy chain are covalently linked by a disulfide linkage.

Monoclonal antibodies against target antigens (e.g., DAXX) are produced by a variety of techniques including conventional monoclonal antibody methodologies such as the somatic cell hybridization techniques of Kohler and Milstein, Nature, 256:495 (1975). Although in some embodiments, somatic cell hybridization procedures are preferred, other techniques for producing monoclonal antibodies are contemplated as well (e.g., viral or oncogenic transformation of B lymphocytes). A preferred animal system for preparing hybridomas is the murine system.

Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the complete human immune system rather than-the mouse system. Splenocytes from the transgenic mice are immunized with the antigen of interest, which are used to produce hybridomas that secrete human mAbs with specific affinities for epitopes from a human protein. (See e.g., WO 91/00906; WO 91/10741; WO 92/03918; WO 92/03917 (each of which is herein incorporated by reference in its entirety); N. Lonberg et al., Nature, 368:856-859 [1994]; L. L. Green et al., Nature Genet., 7:13-21 [1994]; S. L. Morrison et al., Proc. Nat. Acad. Sci. USA, 81:6851-6855 [1994]; Bruggeman et al., Immunol., 7:33-40 [1993]; Tuaillon et al., Proc. Nat. Acad. Sci. USA, 90:3720-3724 [1993]; and Bruggernan et al. Eur. J. Immunol., 21:1323-1326 [1991]).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies. (See e.g., Sastry et al., Proc. Nat. Acad. Sci. USA, 86:5728 [1989]; Huse et al., Science, 246:1275 [1989]; and Orlandi et al., Proc. Nat. Acad. Sci. USA, 86:3833 [1989]). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and the PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies. (See e.g., Larrick et al., Biotechniques, 11:152-156 [1991]). A similar strategy can also be used to amplify human heavy and light chain variable regions from human antibodies (See e.g., Larrick et al., Methods: Companion to Methods in Enzymology, 2:106-110 [1991]).

The term modified antibody is also intended to include antibodies, such as monoclonal antibodies, chimeric antibodies, and humanized antibodies which have been modified by, for example, deleting, adding, or substituting portions of the antibody. For example, an antibody can be modified by deleting the hinge region, thus generating a monovalent antibody. Any modification is within the scope of the technology so long as the antibody has at least one antigen binding region specific.

Chimeric mouse-human monoclonal antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (See e.g., Robinson et al., PCT/US86/02269; European Patent Application 184,187: European Patent Application 171,496; European Patent Application 173,494; WO 86/01533; U.S. Pat. No. 4,816,567; European Patent Application 125,023 [each of which is herein incorporated by reference in its entirety]; Better et al., Science, 240:1041-1043 [1988]; Liu et al., Proc. Nat. Acad. Sci. USA, 84:3439-3443 [1987]; Liu et al., J. Immunol., 139:3521-3526 [1987]; Sun et al., Proc. Nat. Acad. Sci. USA, 84:214-218 [1987]; Nishimura et al., Canc. Res., 47:999-1005 [1987]; Wood et al., Nature, 314:446-449 [1985]; and Shaw et al., J. Natl. Cancer Inst., 80:1553-1559 [1988]).

The chimeric antibody can be further humanized by replacing sequences of the Fv variable region that are not directly involved in antigen binding with equivalent sequences from human Fv variable regions. General reviews of humanized chimeric antibodies are provided by S. L. Morrison, Science, 229:1202-1207 (1985) and by Oi et al., Bio. Techniques, 4:214 (1986). Those methods include isolating, manipulating, and expressing the nucleic acid sequences that encode all or part of immunoglobulin Fv variable regions from at least one of a heavy or light chain.

Suitable humanized antibodies can alternatively be produced by CDR substitution (e.g., U.S. Pat. No. 5,225,539 (incorporated herein by reference in its entirety); Jones et al., Nature, 321:552-525 [1986]; Verhoeyan et al., Science, 239:1534 [1988]; and Beidler et al., J. Immunol., 141:4053 [1988]). All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method that is capable of replacing at least a portion of a CDR of a human antibody with a CDR derived from a non-human antibody. The human CDRs may be replaced with non-human CDRs; using oligonucleotide site-directed mutagenesis.

Also within the scope of the technology are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

The antibodies can be of various isotypes, including, but not limited to: IgG (e.g., IgG1, IgG2, IgG2a, IgG2b, IgG2c, IgG3, IgG4); IgM; IgAQ1; IgA2; IgAsec; IgD; and IgE. In some preferred embodiments, the antibody is an IgG isotype. In other preferred embodiments, the antibody is an IgM isotype. The antibodies can be full-length (e.g., an IgG1, IgG2, IgG3, or IgG4 antibody) or can include only an antigen-binding portion (e.g., a Fab, F(ab′)2, Fv or a single chain Fv fragment).

In preferred embodiments, the immunoglobulin is a recombinant antibody (e.g., a chimeric or a humanized antibody), a subunit, or an antigen binding fragment thereof (e.g., has a variable region, or at least a complementarity determining region (CDR)).

In some embodiments, the immunoglobulin is monovalent (e.g., includes one pair of heavy and light chains, or antigen binding portions thereof). In other embodiments, the immunoglobulin is a divalent (e.g., includes two pairs of heavy and light chains, or antigen binding portions thereof).

The present disclosure further provides pharmaceutical compositions (e.g., comprising one or more of the compounds described above (e.g., a DAXX inhibitor or a compound comprising a structure provided by any of structures I, II, II, IV, or V)). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral, or parenteral. Parenteral administration includes intravenous, intra-arterial, subcutaneous, intraperitoneal, or intramuscular injection or infusion; or intracranial, e.g., intrathecal or intraventricular, administration. In certain embodiments, pharmaceutical compositions (e.g., comprising one or more of the compounds described above (e.g., a DAXX inhibitor or a compound comprising a structure provided by any of structures I, II, II, IV, or V)) are administered by methods that bypass the BBB including, for example, direct application to the surface of the CNS, to the parenchyma of the CNS, to the ventricles of the CNS, and to the cerebrospinal fluid (CSF) of the CNS. In particular, intrathecal and epidural administration may be achieved by single shot, a series of single shots, and/or by continuous administration to the CSF. In certain embodiments, continuous administration to the CSF is provided by a programmable external pump. In other embodiments, continuous administration is provided by a programmable implantable pump.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets, or tablets. Thickeners, flavoring agents, diluents, emulsifiers, dispersing aids or binders may be desirable.

Compositions and formulations for parenteral, intrathecal, or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated (e.g., a neurodegenerative disease (e.g., ALS, FTD)), with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compounds and/or oligonucleotides, and dosages can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g per kg of body weight, once or more daily, to once every 5, 10, or 20 years.

### Examples

Although the disclosure herein refers to certain illustrated embodiments, it is to be understood that these embodiments are presented by way of example and not by way of limitation.

### Methods

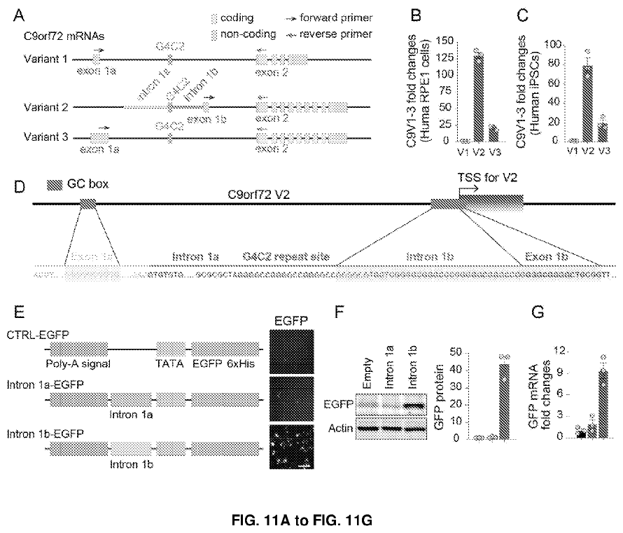

Plasmids—To construct an optogenetic DAXX system, DAXX cDNA from Flag-Daxx/pRK5 (a gift from Xiaolu Yang; Addgene plasmid #27974) (Tang et al., 2006) was subcloned into pHR-mCh-Cry2WT (a gift from Clifford Brangwynne; Addgene plasmid #101221) (Shin et al., 2017) or pHR-sfGFP-Cry2WT using a Gibson Assembly Cloning (NEB, E5510S) method to create pHR-DAXX-mCherry-Cry2WT and pHR-DAXX-sfGFP-Cry2WT. For the promoter activity test, the human C9orf72 intron 1b or intron la sequence (FIG. 11D) was cloned into a pGL4-uPAter-EGFP vector without a promoter 15 element. The PR82 lentiviral expression construct (pLenti-PR82) expressing 82 proline-arginine dipeptide repeats was cloned using the Gateway cloning system into the pLenti-puro-CMV (w118) vector (a gift from Eric Campeau and Paul Kaufman; Addgene plasmid #17452) (Campeau et al., 2009). The PR82 coding sequence was derived from a previous sequence with randomized codons designed to produce only the proline-arginine dipeptide repeat (a gift from Adrian Isaacs) (Mizielinska et al., 2014).

Cell culture, drug treatment, lentiviral shRNA knockdown, and live-cell imaging—HEK293 cells and mouse embryonic fibroblast (MEF) cells were cultured in DMEM containing 10% FBS. Human retinal pigment epithelial 1 (RPE1) cells were cultured in DMEM/F12 containing 10% FBS and 0.01 mg/ml hygromycin B. Human B lymphocytes were cultured in RPMI 1640 medium containing 15% FBS. Human iPSCs were obtained from the NINDS Human Cell and Data Repository and maintained in StemFlex medium (Gibco, A3349401), with medium exchange every other day. All cell lines were checked regularly for mycoplasma contamination. To induce cell stress, RPE1 cells were treated with thapsigargin (30 nM) or tunicamycin (5 μg/ml) for 24 h, and B lymphocytes were treated with thapsigargin (40 nM) or tunicamycin (1 μg/ml) for 24 h. To express PR82, RPE1 cells were transfected with the pLenti-PR82 plasmid and cultured for 48 h.

All knockdown experiments were carried out via transduction with lentiviruses expressing various shRNAs. The shRNAs targeting human DAXX were TRCN0000003800, TRCN0000279733, and TRCN0000003801 (Sigma). Treatments with the various shRNAs yielded similar results, and therefore representative data are presented. To produce lentiviral particles, 5×106 HEK293 cells were seeded onto a 10-cm dish coated with PEI. The next day, the DAXX shRNA-expressing pLKO.1 or pLKO.5 lentiviral plasmid and viral packaging vectors (psPAX2 and pMD2G) were co-transfected using Lipofectamine 2000 (Thermo Fisher, 11668500) for 8 h before the Opti-MEM transfection medium was changed to fresh DMEM containing 10% FBS. After 72 h of lentiviral production, the culture medium was collected and filtered through a 0.45-μm cellulose acetate membrane to remove debris. Lentiviral particles were concentrated by precipitation with 40% PEG 8000 and centrifugation (1,600×g) and then resuspended in lx PBS. The concentrated lentiviral particles were used in the transduction of B lymphocytes or iPSCs for 48 h, followed by puromycin selection (3 μg/ml for B lymphocytes and 0.5-1.5 μg/ml for iPSCs).

For live-cell imaging of Opto-DAXX clustering, 0.8×10⁵ HEK293 cells were seeded onto a FluoroDish (World Precision Instruments, FD35-100) 1 day before transfection with pHR-DAXX-mCherry-Cry2WT or pHR-mCh-Cry2WT. At 24 h after the transfection, live cells were exposed to blue light, and time-lapse images of each channel were captured at the indicated intervals by using an SP8 confocal microscope (Leica). DNA was visualized by DAPI staining in live cells.

Motor neuron differentiation of human iPS cells and experimental analyses—The differentiation of iPSCs into motor neurons was performed as previously described (Liu et al., 2018). In brief, iPSCs were seeded onto Matrigel-coated plates and differentiated into neuroepithelial progenitor cells (NEPCs) by culturing 6 days in neural medium (1:1 DMEM/F12:neurobasal medium. GlutaMax, N2 supplement, B27 supplement, and ascorbic acid) containing 3 μM C1HT199021, 2 μM SB431542, and 2 μM DMH-1. NEPCs were dissociated with dispase (1 U/ml) and split into new plates coated with Matrigel at a ratio of about 1:6. NEPCs were differentiated into motor neuron progenitor cells (MNPCs) using neural medium supplemented with 1 μM CHIR99021, 2 μM SB431542, 2 μM DMH-1, 0.1 μM retinoic acid (RA), and 0.5 μM purmorphamine. After culturing for 6 days, MNPCS were dissociated and cultured in suspension using neural medium containing 0.5 μM RA and 0.1 μM purmorphamine. Six days later, neural spheres were dissociated and plated onto a Matrigel-coated plate. Adherent neural spheres were cultured in neural medium supplemented with 0.5 μM RA, 0.1 μM purmorphamine, and 0.1 μM compound E. After 12 days, mature motor neurons were provided for experiments. The medium was changed every other day during the entire differentiation period.

For knockdown experiments on motor neurons, iMNs were infected twice with the shRNA-expressing lentiviruses, on day 10 and day 12 at the final differentiation stage, and the cells were used on day 15 to allow for efficient DAXX knockdown. Mature iMNs were stressed with tunicamycin (5 μg/ml) in the presence of Na-Phen (10 μM) or 5-aza-2 (2 M) for the indicated times, and neuronal survival at each time point was measured by calcein-AM staining. In brief, iMNs were stained with 3 μM calcein-AM (Invitrogen, C1430), and the fluorescence of surviving iMNs was read using a plate reader (Synergy H1 Hybrid) with excitation/emission at 485 nm/535 nm. Optical fields with calcein-positive iMNs were randomly selected and images captured using a fluorescence microscope (Nikon Eclipse TS100).

C9or172 V2promoleranalysis—The C9orf72 promoter region (Eukaryotic Promoter Database ID C9orf72_1) between −5000 bp to +100 bp was examined for the presence of the GC box motif using the Eukaryotic Promoter Database. Two putative GC boxes, located in introns 1b and la, were identified with a p-value less than 10. To test the promoter activity of the intron 1b and la, HEK293 cells were transfected with pGL4-uPAter-EGFP plasmids containing intron 1b or intron la. One day after the transfection, the cells were analyzed for EGFP expression by western blotting, fluorescence detection, and qPCR analysis. QPCR EGFP expression was normalized using the mRNA of the ampicillin resistance gene (AmpR) from the pGL4-uPAter-EGFP plasmid, together with resident GAPDH mRNA.

SILAC quantitative proteomic analysis—To identify DNA G4C2 repeat-interacting proteins, DNA probes were used to pull down their target proteins in SILAC-labeled cells. The DNA oligonucleotide 5′-(G4C2)eAACAAC-biotin-3′ (SEQ ID NO: 21) and a randomized control, 5′-GACTGACTGATAGATCCTAAGTACTGATTACTGACTAACAAC-biotin-3′ (SEQ ID NO: 23), together with their complementary oligonucleotides without a biotin label were synthesized (Integrated DNA Technologies). Each pair of complementary strands were incubated at 95° C. for 5 min and ramped down to 20° C. in 5° C./min increments to anneal in 2× annealing buffer (20 mM Tris-HCl [pH 8.0], 100 mM NaCl, 2 mM EDTA). Annealed dsDNAs were phosphorylated at 37° C. for 2 h using T4 polynucleotide kinase (NEB, M0201S) and then ligated with T4 DNA ligase at room temperature for 4 h. The dsDNAs were purified via phenol-chloroform extraction and stored at −20° C.

HEK293 cells were cultured in SILAC medium with either light lysine and arginine, or heavy lysine (¹³C₆ ¹⁵N₂) and arginine (¹³C₆ ¹⁵N₄), along with 10% dialyzed fetal bovine serum (Thermo Fisher Scientific, 88440) and penicillin/streptomycin. Following 10 days of metabolic labelling, nuclear proteins were extracted using Nuclear and Cytoplasmic Extraction Reagent (ThermoFisher Scientific). Biotin-labeled G4C2 repeats or random dsDNAs (20 μg each) were incubated with streptavidin beads (Dynabeads MyOne Streptavidin C1, Invitrogen, 65001) in DNA binding buffer (2 M 10 NaCl, 10 mM Tris-HCl [pH 7.5], 1 mM EDTA, 0.01% Tween-20) at room temperature for 1 h with rotation. The dsDNA-bead complexes were washed twice using DNA binding buffer and then twice with protein binding buffer (PBS [pH 7.4] containing 0.01% Tween-20). The dsDNA-bead probes were then incubated with 400 g of nuclear protein in protein binding buffer at 4° C. for 2 h with rotation, washed three times with protein binding buffer, and heated at 95° C. for 5 min in NuPAGE loading buffer (Invitrogen, NP0007) containing 20 mM DTT. The resulting immunoprecipitates were separated on a 4-12% gradient gel and digested with trypsin. The digested peptide samples were analyzed and quantified with an LTQ-Orbitrap-Velos mass spectrometer.

Protein purification and EMSA—DAXX protein was expressed and purified from human cells. IIEK293 cells were transfected with Flag-DAXX-pRK5 (Addgene 27974) and then lysed by sonication in ice-cold lysis buffer (20 mM Tris-HCl [pH 7.5], 150 mM NaCl, 1 mM Na₂EDTA, 1 mM EGTA, 1% Triton X-100, 2.5 mM sodium pyrophosphate, 1 mM beta-glycerophosphate, 1 mM Na₃VO₄, 1 μg/ml leupeptin, 1 mM PMSF (APExBIO, A2587), and Protease Inhibitor Cocktail (1:200, Millipore Sigma, P8340) with a Diagenode Bioruptor at high power with an on/off cycle of 30 sec for 20 min. Cell lysates were centrifuged at 12,000×g at 4° C. for 20 min, and the supernatants were harvested for immunoprecipitation. After incubation with anti-Fla^{g} M2 magnetic beads (Sigma, M8823) at room temperature for 1 h, the beads were washed three times with a buffer (50 mM Tris-HCl [pH 7.4] and 150 mM NaCl) and then eluted with a buffer containing 50 mM Tris-HCl (pH 7.4), 150 mM NaCl, and 150 ng/μl 3×Flag peptides (Sigma, F4799) at 4° C. for 30 min with rotation. Purified protein was concentrated with a centrifugal filter (Millipore, 50 KDa, UFC805096) and stored at −80° C. in buffer containing 20 mM Tris-HCl (pH 7.5), 150 mM NaCl, 1 mM DTT, 50% glycerol, 1 mM PMSF (APExBIO, A2587), and Protease Inhibitor Cocktail (1:200, Millipore Sigma, P8340).

For EMSA analysis, a ssDNA probe, (CCCCGG)₁₀ (SEQ ID NO: 17), and a size-matched control probe (SEQ ID NO: 19):

GACTGACTGATAGATCCTAAGTACTGATTACTGACTATAGATCTAAGTCA
TGATCAGTTA

were synthesized and labeled with an Alexa Fluor 488 fluorescent tag at the 5′ terminus (Integrated DNA Technologies). To generate dsDNA probes, the fluorescence-labeled ssDNA probe was annealed with its complementary strand (C4C2 probe, SEQ ID NO: 18; control, SEQ ID NO: 20). Increasing concentrations of purified protein were 10 incubated with the probes (50 nM) in binding buffer containing 10 mM Tris (pH 7.5), 1 mM EDTA, 0.1 mM DTT, 10 μg/ml BSA, and 5% glycerol. After incubation at room temperature for 30 min, the mixture was run on a non-denaturing polyacrylamide gel in TBE buffer. Gel shift images were captured on an Amersham Typhoon Imager 9200, and the ratio of bound DNA to free DNA was analyzed with ImageJ (version 2.0.0-rc-59/1.51k).

ATAC-PALM—HEK293 cells were plated onto 8-well Lab-TEK chambers (Thermo Fisher #155409) pre-coated with fibronectin (Millipore FC010, 7.5 μl/ml). At 24 h after plating, cells were transfected with pHR-DAXX-sfGFP-Cry2WT using Lipofectamine 3000 (Thermo Fisher, L3000001), incubated for 48 h, and light-activated by a blue-light LED transilluminator (BLooK, GeneDireX, model: BK001) for 5 min before fixation in 4% paraformaldehyde solution (Electron Microscopy Sciences #15710) for 10 min at room temperature. Cells were washed, permeabilized, and incubated with Tn5 PA-JF549 transposase as described (Xie et al., 2020). For a single well of the 8-well Lab-TEK chamber, a 20-μl reaction mix (10 mM Tris-HCl [pH 7.6], 5 mM MgCl₂, 10% dimethylformamide, and 25 nM Tn5 PA-JF549 transposase) was spread over the entire well using a sheet of Parafilm, and the cells were incubated at 37° C. for 1 h. Following the incubation with the transposase, the cells were washed 3 times with 1×PBS containing 0.01% SDS and 50 mM EDTA for 8 min each at 55° C., then washed 2 times with 1×PBS, and kept in 1×PBS during imaging.

2D ATAC-PALM single-molecule imaging was performed on a Nikon Eclipse Ti microscope equipped with a 100× oil-immersion objective lens (Nikon, N. A.=1.49), a Lumencor light source, two filter wheels (Lambda 10-3, Sutter Instrument, Novato, CA), perfect focusing systems, and EMCCD (iXon3, Andor, Belfast, United Kingdom). Emission filters (Semrock, Rochester, New York) were switched in front of the cameras for GFP or JF549 emission, and a band mirror (405/488/561/633 BrightLine quad-band bandpass filter, Semrock) was used to reflect the laser into the objective. Cells were first excited with a 10% 488 nm laser to acquire an epifluorescence GFP channel image. Then Tn5 PA-JF549 transposase single molecules were detected using a 405-nm laser (10% power) for photo-activation and a 561-nm laser (100% power) for excitation. The acquisition time was 30 ms. A given plane of the cell was imaged for 10,000-20,000 iterations to exhaust single-molecule detections.

Single molecules were localized using the ThunderSTORM plugin of ImageJ and visualized using the Normalized Gaussian method. To calculate the Tn5 PA-JF549 transposase localization density per Opto-GFP-DAXX droplet in the light-activated sample, a GFP channel image of the same cell was first used to select droplets as regions of interest (ROIs). For localizations, 20× approximately 1-μm² areas of the nucleus were randomly chosen and stored as ROIs. Localization density per ROI was calculated by dividing the number oflocalizations per ROI by tie area of the ROI. Localization density was normalized by the total number of localizations per nucleus, accounting for cellular variation in Tn5 PA-JF549 labeling.

ATAC-seq—Human B lymphocytes (approximately 1×10⁵) or progenitor-differentiated motor neurons (approximately 1.6×10⁶) were used for ATAC-seq as described (Buenrostro et al., 2015). Cells were pelleted in 1×PBS (500×g for 5 min, 4° C.) and resuspended in 50 μl of cold lysis buffer (10 mM Tris-IICl [pII 7.4], 10 mM NaCl, 3 mM MgCl₂, and 0.1% IGEPAL CA-630) with gentle pipetting. Cell lysates were centrifuged (500×g for 10 min, 4° C.) to pellet the nuclei, which were then incubated with 50 μl of transposition reaction buffer (25 μl of 2× Tagment DNA reaction buffer [Illumina FC-121-1030], 2.5 μl of Nextera Tn5 Transposase [Illumina FC-121-1030], and 22.5 μl of nuclease-free H₂O) at 37° C. for 30 min with gentle rotation. After the reaction, genomic DNA was purified with a Qiagen MinElute PCR Purification Kit (Qiagen, 28204) and eluted in 10 μl of elution buffer (10 mM Tris buffer [pH 8.0]).

To amplify the transposed DNA fragments, the DNA sample was mixed with PCR amplification buffer containing nuclease-free H₂O, Nextera PCR Primer 1 (SEQ ID NO: 25), Nextera PCR Primer 2 (barcode) (SEQ ID NO: 26), and NEBNext High-Fidelity 2×PCR Master Mix (New England Labs, M0541), and then amplified by 1 cycle of 72° C. incubation for 5 min and 98° C. for 30 see, followed by 5 cycles of 98° C. for 10 sec, 63° C. for 30 sec, and 72° C. for 1 min. To avoid PCR saturation of a library and reduce the GC and size bias, 5 μl of the first PCR reaction were amplified in a side qPCR reaction to select an appropriate cycle number for the second-round PCR reaction, which was set to 1 cycle of 98° C. incubation for 30 sec, followed by 20 cycles of 98° C. for 10 sec, 63° C. for 30 sec, and 72° C. for 1 min. The cycle number corresponding to a quarter of the maximum fluorescence intensity was used in the final PCR amplification. After the final amplification, PCR products were purified using a Qiagen MinElute PCR Purification Kit (Cat. NO. 28204) and analyzed by deep sequencing.

ATAC-seq data were processed on Galaxy (Batut et al., 2018). In brief, adapters were removed using Cutadapt and only reads with a length of <20 nt were retained for analysis. The quality of the reads was measured by FastQC. The trimmed reads were sequentially mapped to the human reference genome (hg39 version) using Bowtie in an end-to-end model. The maximum fragment length (distance between read pairs) was set to 1000 bp, and the parameters were set as highly sensitive. Reads with low mapping quality, inappropriate pairing, or mapping to mitochondrial DNA were filtered out. Duplicate fragments were removed with Picard MarkDuplicates. An insert size of Tn5 was plotted with a paired-end histogram for fragment length distribution, with peaks at 200 bp, 400 bp, and/or 600 bp. MACS2 was used to find peak callings (extension size, 200 and shift size, 100), and the MACS2 file was used to generate a bigwig and heatmap of coverage at TSSs of interest.

HiChIP—HiChIP was performed as previously described (Mumbach et al., 2016). IIEK293 cells were plated in 10-cm dishes 1 day before pIIR-DAXX-mCherry-Cry2WT transfection, followed by a 2-day incubation. The plates with >80% of cells expressing Opto-DAXX were randomly divided into two groups, with each containing 1×10⁷ cells. One group was activated by blue-light illumination for 10 min, and the other served as a negative control. Cells were detached, pelleted, resuspended at 1×10⁶ cells/ml in freshly prepared 1% formaldehyde, and incubated at room temperature for 10 min with gentle rotation, with constant blue-light illumination for the activated group during this step. The formaldehyde was quenched with glycine at a final concentration of 125 mM for 5 min. Following three washes with ice-cold PBS, 1×10⁷ cells were lysed in 500 μl ice-cold lysis buffer (10 mM Tris-HCl [pH 8.0], 10 mM NaCl, 0.2% NP-40, and Protease Inhibitor Cocktail [1:200, Millipore Sigma, P8340]), incubated at 4° C. for 30 min, and centrifuged at 2,500×g for 5 min to collect nuclei. The nuclear pellets were washed once with 500 μl of ice-cold lysis buffer, suspended in 100 μl 0.5% SDS, and incubated at 62° C. for 10 min. Triton X-100 (10%, 50 μl) was added into the pellets to quench the SDS, followed by the addition of 285 μl H2O and incubation at 37° C. for 15 min. In situ chromatins were digested with 375 U of MboI restriction enzyme in 500 μl of 1×NEB buffer 2 at 37° C. for 2 h before heat inactivation (65° C., 20 min). The overhangs of the cut DNA fragments were filled in with biotin-labeled dATP (Thermo, 19524016) using 5 U DNA polymerase I large (Klenow) fragment (NEB, M0210) at 37° C. for 1 h. The blunt-ended DNA fragments were ligated with NEB T4 DNA ligase buffer, 10% Triton X-100, BSA, and T4 DNA ligase (NEB. M0202) at room temperature for 4 h with rotation. After the incubation, nuclei were pelleted at 2500×g for 5 min and subjected to sonication (Diagenode Bioruptor: 30-see cycles, high power for 15 min) in nuclear lysis buffer (50 mM Tris-HCl [pH 7.5], 10 mM EDTA, 1% SDS, and Protease Inhibitor Cocktail [1:200, Millipore Sigma, P8340]). The sonicated samples were centrifuged at 16,100×g for 15 min at 4° C. and used for chromatin immunoprecipitation with an antibody against DAXX (Sigma. D7810), following the same protocol described in the section on chromatin immunoprecipitation qPCR herein. A sample (150 ng) of the DNA pulled down from the ChIP was used to capture biotin-labeled DNAs using 5 μl of Streptavidin C-1 beads in 20 μl of 1× biotin binding buffer (5 mM Tris-HCl [pH 7.5], 0.5 mM EDTA, and 1 M NaCl) at room temperature for 15 min with rotation. Beads were washed twice with 500 μl Tween wash buffer (5 mM Tris-HCl [pH 7.5], 0.05% Tween-20, 0.5 mM EDTA, and 1 M NaCl) at 55° C. for 2 min with shaking and once with 100 Ip of 2× Tagment DNA buffer (20 mM Tris-HCl [pH 7.5], 10 mM MgCl₂, and 20% dimethylformamide). The washed beads were added into 25 μl of 2× Tagment DNA buffer with 4 μl Tn5 Transposase (Illumina, FC-121-1030) and incubated at 55° C. for 10 min. After the incubation, the beads were removed and washed twice in 50 mM EDTA at 50° C. (first for 30 min and then 3 min), twice in Tween wash buffer at 55° C. for 2 min, and once in 10 mM Tris. The washed beads were used for PCR amplification and sequencing as described in the section on ATAC-seq.

For HiChIP data analysis, Illumina and Nextera adapter sequences were removed using Trim Galore (Felix Krueger, 2021) (version 0.6.6). The raw reads were aligned to the human hg19 genome using HiC-Pro (Servant et al., 2015) (version 2.11.4) with bin sizes of 5 kb, 50 kb, and 100 kb. The MboI restriction site, followed by fill-in, and the ligation was specified as GATCGATC (SEQ ID NO: 33). The processed data were analyzed, and contact maps were generated using the HiTC R package (Servant et al., 2012) (version 1.23.0).

The processed BAM files and peak files from the H3K27ac experiments in HEK293 cells were downloaded directly from ENCODE website (Consortium, 2004). The raw fastq file of Myc-DAXX ChIP-seq experiments in HEK293 was downloaded from Gene Expression Omnibus (GEO) with accession code GSE107348. The raw reads were aligned to the human hg19 genome using Bowtie 2 (Langmead and Salzberg, 2012) (version 2.3.5.1).

Chromatin immunoprecipitation qPCR—ChIP-qPCR analysis of RPE1 cells, B lymphocytes, and iPSC-differentiated motor neurons was performed by using a CHIP assay kit (Millipore Sigma, 17-295). In brief, protein complexes were crosslinked with 1% formaldehyde at 37° C. for 10 min and quenched with glycine at a final concentration of 125 mM at 37° C. for 5 min. After three washes with cold 1×PBS containing 1 mM 10 PMSF (APExBIO, A2587) and Protease Inhibitor Cocktail (1:200, Millipore Sigma, P8340), the cells were pelleted and lysed in SDS lysis buffer (50 mM Tris [pH 8. 1], 1% SDS, 10 mM EDTA, Protease Inhibitor Cocktail [1:200, Millipore Sigma, P8340], and 1 mM PMSF [APExBIO, A2587]). The cell lysates were sonicated on ice to achieve an average DNA length of approximately 200 bp to approximately 1000 bp. After centrifugation, the supernatants were harvested and diluted 10 times with buffer (0.01% SDS, 1.1% Triton X-100, 1.2 mM EDTA, 16.7 mM Tris-HCl [pH 8.1], 167 mM NaCl, Protease Inhibitor Cocktail [1:200, Millipore Sigma, P8340], and 1 mM PMSF [APExBIO, A2587]) and pre-cleared with salmon sperm DNA/Protein A agarose-50% slurry (Millipore Sigma, 16-157C) at 4° C. for 30 min. The pre-cleared samples were incubated with antibodies against RNA polymerase II (Millipore Sigma, 17-620), DAXX (Sigma, D7810), or IgG (Millipore Sigma, 17-620; Cell Signaling Technology, 2729) at 4° C. overnight with rotation. The beads were washed sequentially with low-salt immune complex wash buffer (0.1% SDS, 1% Triton X-100, 2 mM EDTA, 20 mM Tris-HCl [pH 8.1], and 150 mM NaCl), high-salt immune complex wash buffer (0.1% SDS, 1% Triton X-100, 2 mM EDTA, 20 mM Tris-HCl [pH 8.1], and 500 mM NaCl), LiCl salt immune complex wash buffer (0.25 M LiCl, 1% IGEPAL-CA630, 1% sodium deoxycholate, 1 mM EDTA, and 10 mM Tris [pH 8.1]), and TE buffer (10 mM Tris [pH 8.0] and 1 mM EDTA). Beads were eluted with elution buffer (1% SDS and 0.1M NaHCO₃) at room temperature for 15 min with rotation. NaCl (20 μl, 5M) and RNase A (2 μl of 10 mg/ml, ThermoFisher Scientific, EN0531) were added to 500 μl of each eluate and incubated overnight at 65° C. with rotation. Proteinase K (2 μl of 20 mg/ml, ThermoFisher Scientific, E00491) was added and incubated at 60° C. for 1 h with rotation. DNA was purified using the phenol-chloroform method. Two primer sets were designed for C9orf72 intron 1b:

Primer set 1:
SEQ ID NO: 5
5′-TCTGGAACTCAGGAGTCGCG-3′ (forward 1)
SEQ ID NO: 6
5′-GCAGCCGAACCCCAAACAGC-3′ (reverse 1)
Primer set 2
SEQ ID NO: 7
5′-TGGCGAGTGGGTGAGTGAGG-3′ (forward 2)
SEQ ID NO: 8
5′-GAGGGAAAGTAAAAATGCGTCGA-3′ (reverse 2)

Subsequently, qPCR analysis was performed, and fold changes were normalized with IgG controls and inputs in statistical analysis.

Fluorescence in situ hybridization and co-immunostaining—DNA FISH was performed with modifications of a previously described method (Chaumeil et al., 2013). (CCCCGG)₄ (SEQ ID NO: 15) with an Alexa Fluor 488 fluorescence tag at the 3′ terminus was synthesized (Integrated DNA Technologies) and used as a probe for G4C2 repeats as previously reported (Renton et al., 2011). Cells were fixed with 2% paraformaldehyde in 1×PBS (pH 7.4) at room temperature for 15 min, washed, and permeabilized with ice-cold 0.4% Triton X-100/1×PBS for 10 min. Cells were blocked in buffer containing 2.5% BSA, 10% goat serum, and 0.1% Tween-20 at room temperature for 1 h, incubated with anti-DAXX antibody (Cell Signaling Technology, 4533) at 4° C. overnight, and with a fluorescent secondary antibody at room temperature for 2 h. Cells were treated with RNase A (0.1 μg/ul) at 37° C. for 1 h, washed, and permeabilized with ice-cold 0.7% Triton X-100 and 0.1 M HCl for 10 min. Genomic DNA and the probe were denatured in 50% formamide, 2×SSC, and 10% dextran sulfate at 95° C. for 30 min, incubated at 37° C. for 1 h, and washed with 0.4×SSC and 0.3% Tween-20 at room temperature. Slides were stained with DAPI and sealed for imaging.

RNA FISH for foci containing G4C2 repeat RNAs was performed as previously described (Zu et al., 2013). In brief, C9HRE iMNs were fixed in 3.75% formaldehyde in PBS at room temperature for 10 min and permeabilized in prechilled 70% ethanol on ice for 30 min. Cells were rehydrated in wash buffer (40% deionized formamide in 2×SSC) for 10 min, followed by rehydration with 40% formamide in 2×SSC for 10 min. After blocking in hybridization buffer (40% formamide, 2×SSC, 20 μg/ml BSA, 100 mg/ml dextran sulfate, 10 μg/ml yeast tRNA, and 2 mM vanadyl sulfate ribonucleosides) at 55° C. for 10 min, cells were incubated at 55° C. for 2 h in the hybridization buffer containing 125 nM (C4G2)4-Cy3 (SEQ ID NO: 16) probes, which had been denatured at 95° C. for 5 min and chilled on ice. Cells were washed three times with the wash buffer at 55° C. for 10 min and sealed with Prolong Gold Antifade reagent containing DAPI (Invitrogen, P36931). All DNA and RNA FISH images were captured using an SP8 confocal microscope (Leica).

Immunostaining and nascent RNA visualization—Cells were seeded onto a glass slide coated with PEI, cultured for 24 h, washed, and fixed with 4% formaldehyde for 20 min at room temperature. After washes, the cells were permeabilized and blocked in buffer containing 5% normal serum and 0.3% Triton X-100 in 1×PBS for 2 h at room temperature, followed by incubation with primary antibody: anti-DAXX (Cell Signaling Technology, 4533), anti-PML (Santa Cruz, se-966), anti-ATRX (Santa Cruz, sc55584), anti-HDAC1 (Santa Cruz, sc81598), anti-H3K9me3 (Active Motif, 61013), anti-RNA polymerase II (Cell Signaling Technology, 2629), or anti-ChAT (Millipore Sigma. AB144P) at 4° C. overnight. The slides were washed and incubated with a fluorescent secondary antibody at room temperature for 2 h, followed by PBS washes. The slides were then sealed with a Prolong Gold Antifade reagent with DAPI (Invitrogen, P36931). All fluorescent images were captured with an SP8 confocal microscope (Leica). Puncta were quantified using ImageJ and statistically analyzed.

Nascent RNAs in HEK293 cells were labeled using an Alexa Fluor 488-tagged nucleoside and click chemistry as described (Invitrogen, C10329). In brief, HEK293 cells were plated onto glass slides and the transfected with Opto-DAXX the next day. One day after transfection, the cells were exposed to blue light for 4 h. During the last hour, the cells were incubated with 5-ethynyl uridine to label nascent RNAs, then fixed and permeabilized, and subjected to click chemistry to visualize fluorescently labeled nascent RNAs using an SP8 confocal microscope (Leica). Finally, the fluorescence intensity of 5-ethynyl uridine inside or outside the DAXX droplets was measured and analyzed.

Quantitative PCR— Total RNAs were extracted with an RNeasy Plus mini kit (Qiagen, 74136), and cDNAs were synthesized using QuantiTect reverse transcription reagents (Qiagen, 205313). qPCR reactions were carried out on a Bio-Rad thermal cycler using PowerUp SYBR Green Master Mix (ThermoFisher Scientific). The primer sets for C9orf72 transcripts V1, V2, and V3 were described previously (Gendron et al., 2017, incorporated herein by reference). The expression of C9orf72 pre-mRNA was measured with a pair of primers targeted to the junction region between exon 2 and intron 3 (SEQ ID NO: 11 and 12). The mRNA expression levels were analyzed by the ΔΔCt method and normalized against housekeeping genes. In the promoter analysis, the EGFP transcription level was normalized to the GAPDH transcript and the plasmid-encoded ampicillin resistance (AmpR) transcript to exclude any bias induced by differential transfection efficiency.

Immunoblotting—Cultured cells were washed twice with PBS and lysed in cold RIPA buffer containing 50 mM Tris (pH 7.5), 0.5% SDS, 150 mM NaCl, 0.5% NP40, 20 mM EDTA, 1 mM PMSF, and Protease Inhibitor Cocktail (1:200, Millipore Sigma, P8340).

Cytoplasmic and nuclear fractions of B lymphocytes were isolated with a subcellular fractionation kit (Thermo Fisher, 78840). Human spinal cord tissues were homogenized and lysed in a modified RIPA buffer (50 mM Tris [pH 7.5], 150 mM NaCl, 1% NP40, 0.1% SDS, 100 mM NaF, 17.5 mM 8-glycerophosphate, 2.5% sodium deoxycholate, and 10% glycerol) containing phosphatase inhibitors 2 and 3 (1:100; Millipore Sigma), 1 mM PMSF, 2 mM NaVO₄, and Protease Inhibitor Cocktail (1:200, Millipore Sigma, P8340). After sonication on ice, the samples were centrifuged at 12,000×g for 10 min at 4° C., and supernatants were collected for analysis.

Protein concentrations were measured by the bicinchoninic acid assay (ThermoFisher, 23225). Following SDS-PAGE and western blotting, membranes were incubated with primary antibodies at 4° C. overnight, including anti-DAXX (Cell Signaling Technology, 4533), anti-C9orf72 (BioRad, VMA00065), anti-suv39h1 (Cell Signaling Technology, D11B6), anti-ATRX (Santa Cruz, sc55584), anti-IIDACI (Santa Cruz, sc81598), anti-H3K9me3 (Active Motif, 61013), anti-H3K27ac (Cell Signaling Technology, 8173), anti-H3 (Cell Signaling Technology, 4499), anti-PARP (Cell Signaling Technology, 9542), anti-GFP (Invitrogen, A-11122), anti-Flag (Sigma, F3165), anti-GAPDH (Tnvitrogen, TAB1001), and anti-actin (Santa Cruz, se-47778). After washes with TBST, the membranes were incubated with a fluorescent secondary antibody at room temperature for 2 h. Western blot images were captured by an Odyssey scanner (LI-COR) and analyzed with Image Studio (version 5.2.5). Protein expression levels were normalized with actin for the total protein, GAPDH for the cytoplasmic fraction, and PARP for the nuclear fraction.

Statistic analysis—Experiments on human RPE1s, B lymphocytes, HEK293 cells, iPSCs, and iPSC-differentiated motor neurons were performed with triplicate samples in at least three independent experiments. Experiments on iPSC-differentiated motor neurons were repeated on three independent cell lines for each group of control and patient cells. Average values for each group were used for statistical analysis. All image analysis was performed in a blinded manner. Statistical analysis was performed on Prism 9.0 software. The unpaired Student's t-test was applied for single comparisons, and one-way analysis of variance (ANOVA) for multiple comparisons with Bonferroni's test. All data are presented as means±SEM unless otherwise indicated.

Oligonucleotides—Oligonucleotides having the following nucleotide sequences (provided 5′ to 3′) and modifications (where indicated) were used in experiments during the development of embodiments of the technology described herein.

C9mRNA V1-Forward
(SEQ ID NO: 1)
CCACGTAAAAGATGACGCTTGATA
C9mRNA V2-Forward
(SEQ ID NO: 2)
CGGTGGCGAGTGGATATCTC
C9mRNA V3-Forward
(SEQ ID NO: 3)
GCAAGAGCAGGTGTGGGTTT
C9mRNA Reverse for V1-3
(SEQ ID NO: 4)
TGGGCAAAGAGTCGACATCA
C9V2 Promoter / C9 Intron 1b (Forward 1)
(SEQ ID NO: 5)
TCTGGAACTCAGGAGTCGCG
C9V2 Promoter / C9 Intron 1b (Reverse 1)
(SEQ ID NO: 6)
GCAGCCGAACCCCAAACAGC
C9V2 Promoter / C9 Intron 1b (Forward 2)
(SEQ ID NO: 7)
TGGCGAGTGGGTGAGTGAGG
C9V2 Promoter / C9 Intron 1b (Reverse 2)
(SEQ ID NO: 8)
GAGGGAAAGTAAAAATGCGTCGA
AmpR-Forward
(SEQ ID NO: 9)
CACCTATCTCAGCGATCTGTCT
AmpR-Reverse
(SEQ ID NO: 10)
GTTGCAGGACCACTTCTGCG
Junction of C9 Exon2 and Intron3 (Forward)
(SEQ ID NO: 11)
CCGGAAAGGAAGAATATGGA
Junction of C9 Exon2 and Intron3 (Reverse)
(SEQ ID NO: 12)
CATTTATTGTTTGATGTTCACTGC
Human GAPDH (Forward)
(SEQ ID NO: 13)
AAGGTGAAGGTCGGAGTCAAC
Human GAPDH (Reverse)
(SEQ ID NO: 14)
GGGGTCATTGATGGCAACAATA
C9HRE DNA Probe (for DNA FISH)
(SEQ ID NO: 15)
(CCCCGG)4-Alexa 488
CCCCGG CCCCGG CCCCGG CCCCGG-Alexa 488
C9HRE RNA Probe (for RNA FISH)
(SEQ ID NO: 16)
(CCCCGG)4-Cy3
CCCCGG CCCCGG CCCCGG CCCCGG-Cy3
C9HRE DNA Probe (Forward)(for EMSA)
(SEQ ID NO: 17)
(GGGGCC)10-Alexa 488
Alexa488-GGGGCC GGGGCC GGGGCC GGGGCC GGGGCC GGGGCC GGGGCC
GGGGCC GGGGCC GGGGCC
C9HRE DNA Probe (Reverse)(for EMSA)
(SEQ ID NO: 18)
(GGCCCC)10
GGCCCC GGCCCC GGCCCC GGCCCC GGCCCC GGCCCC GGCCCC GGCCCC GGCCCC
GGCCCC
Control DNA Probe (Forward)(for EMSA)
(SEQ ID NO: 19)
GACTGACTGATAGATCCTAAGTACTGATTACTGACTATAGATCTAAGTCATGATCAGTTA
Control DNA Probe (Reverse)(for EMSA)
(SEQ ID NO: 20)
TAACTGATCATGACTTAGATCTATAGTCAGTAATCAGTACTTAGGATCTATCAGTCAGTC
C9HRE DNA Probe (Forward)(Proteomics analysis)
(SEQ ID NO: 21)
(GGGGCC)6AACAAC-biotin
GGGGCC GGGGCC GGGGCC GGGGCC GGGGCC GGGGCC AACAAC-biotin
C9HRE DNA Probe (Reverse)(Proteomics analysis)
(SEQ ID NO: 22)
GTTGTT (GGCCCC)6
GTTGTT GGCCCC GGCCCC GGCCCC GGCCCC GGCCCC GGCCCC
Control DNA Probe (Forward)(Proteomics analysis)
(SEQ ID NO: 23)
GACTGACTGATAGATCCTAAGTACTGATTACTGACTAACAAC-biotin
Control DNA Probe (Reverse)(Proteomics analysis)
(SEQ ID NO: 24)
GTTGTTAGTCAGTAATCAGTACTTAGGATCTATCAGTCAGTC
Nextera Primer 1 (For ATAC-seq)
(SEQ ID NO: 25)
CAAGCAGAAGACGGCATACGAGATAGGAGTCCGTCTCGTGGGCTCGGAGATGT
Nextera Primer 2 (Barcode 1, for ATAC-seq)
(SEQ ID NO: 26)
ACATCTCCGAGCCCACGAGACTAAGGCGAATCTCGTATGCCGTCTTCTGCTTG
Nextera Primer 2 (Barcode 2, for ATAC-seq)
(SEQ ID NO: 27)
ACATCTCCGAGCCCACGAGACCGTACTAGATCTCGTATGCCGTCTTCTGCTTG
Nextera Primer 2 (Barcode 3, for ATAC-seq)
(SEQ ID NO: 28)
ACATCTCCGAGCCCACGAGACGGACTCCTATCTCGTATGCCGTCTTCTGCTTG
Nextera Primer 2 (Barcode 4, for ATAC-seq)
(SEQ ID NO: 29)
ACATCTCCGAGCCCACGAGACTAGGCATGATCTCGTATGCCGTCTTCTGCTTG
Nextera Primer 2 (Barcode 5, for ATAC-seq)
(SEQ ID NO: 30)
ACATCTCCGAGCCCACGAGACCTCTCTACATCTCGTATGCCGTCTTCTGCTTG
Nextera Primer 2 (Barcode 6, for ATAC-seq)
(SEQ ID NO: 31)
ACATCTCCGAGCCCACGAGACCAGAGAGGATCTCGTATGCCGTCTTCTGCTTG

### Example 1—DNA-Binding Protein DAXX Marks Nuclear Pathology in C9Orf72 HRE ALS Patient Cells

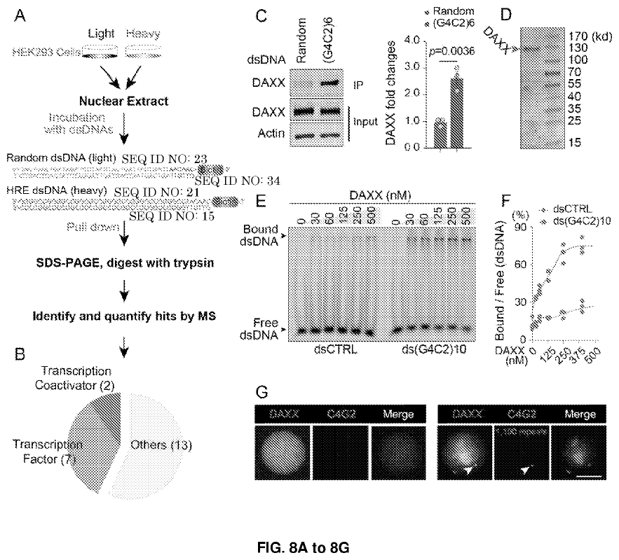

During the development of embodiments of the technology provided herein, experiments were conducted to identify DNA-binding proteins (DBP) specifically associated with the C9orf72 HRE (“C9HRE”) DNA. These experiments employed a quantitative proteomic analysis using stable isotope labeling with amino acids (SILAC). HEK293 cells were metabolically labeled with SILAC isotopes to saturation and then lysed and subjected to a pull-down assay using biotinylated double-stranded DNAs (dsDNA) of (G4C2)₆ or a length-matched random sequence control (FIG. 8A). Subsequent mass spectrometry analysis identified 309 proteins in the precipitates isolated by the (G4C2)₆ probes. Among them, 22 proteins were found to be highly enriched by the C9HRE DNA, of which 9 are transcriptional factors (FIG. 8B), indicating a successful pull-down of DNA-binding proteins. The most enriched protein that selectively recognized C9HRE DNA, as ranked by SILAC ratio, was DAXX, an epigenetic factor involved in transcriptional regulation (He et al., 2015; Lin et al., 2006).

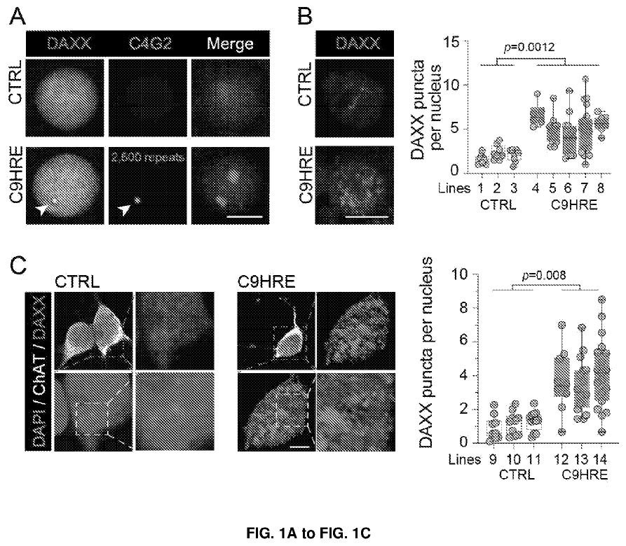

Several assays were used to validate the interaction between DAXX and the G4C2 repeat dsDNA. First, experiments were conducted to confirm that DAXX was significantly enriched in the nuclear precipitates pulled down by the (G4C2)₆ dsDNA probe in an immunoblotting assay (FIG. 8C). Next, experiments were conducted to test whether DAXX protein and G4C2 repeat dsDNA interact directly. In these experiments, human DAXX protein (FIG. 8D) was purified and an electrophoresis mobility shift assay (EMSA) was performed using 5′ AlexaFluor 488-labeled (G4C2)₁₀ dsDNA or a length-matched random sequence control. The EMSA results demonstrated a significantly preferential binding of DAXX to (G4C2)₁₀ dsDNA when compared to the random control probe (FIG. 8E, F), confirming specific recognition of the C9orf72 DNA repeat by DAXX. In addition, experiments were performed to establish the association of endogenous DAXX with the expanded C9orf72 (G4C2)n DNA repeats in situ. In these experiments, DNA fluorescent in situ hybridization (FISH) was used to immunostain DAXX on multiple lines of ALS and/or FTD patient lymphocytes carrying C9orf72 HRE mutations. A co-localization between the DAXX immunostaining and FISH staining at the C9HRE locus was indicated by a well-demarcated punctum present only in the nuclei of patient cells and not in the control cells (FIG. 1A).

In addition to DAXX accumulation at the expanded G4C2 repeat locus, the data indicated an aberrant distribution pattern of DAXX throughout the nuclei in the C9HRE patient cells. Immunostaining of endogenous DAXX in multiple lines of lymphocytes or induced pluripotent stem cell-differentiated motor neurons (iMNs) showed significantly increased DAXX signals and enlarged DAXX-positive granules in the C9HRE cells when compared to those in control cells (FIG. 1B, C). In the control B lymphocytes and iMNs, the signals for DAXX mainly showed a diffuse pattern or occasionally appeared as small faint dots in both the nucleus and cytoplasm (FIG. 1B, C). However, in the C9HRE cells, the abnormally accumulated DAXX was preferentially localized to the nucleus, where the DAXX granules were either isolated or connected into a network, indicating widespread nuclear changes in the patient cells (FIG. 1B, C).

### Example 2—Nuclear Phase Separation of DAXX Remodels Chromatin Structure

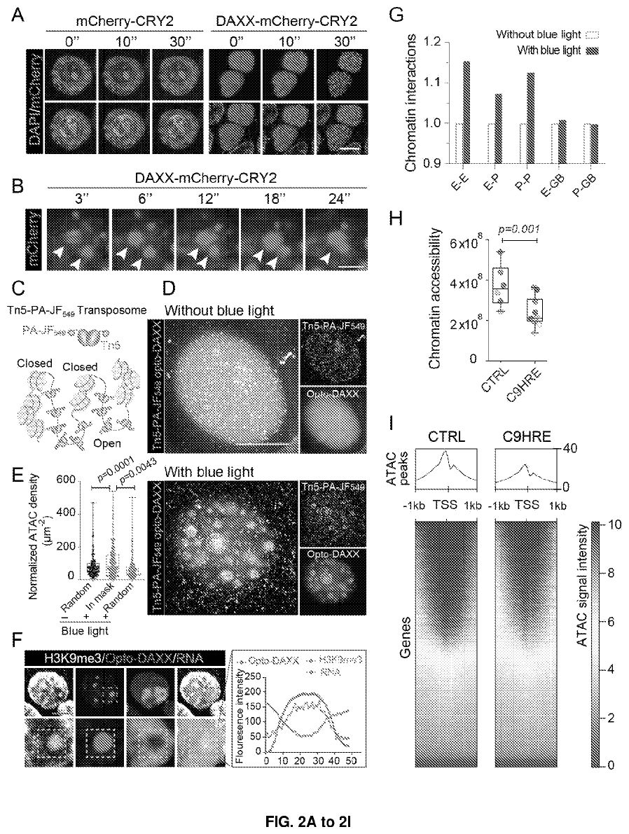

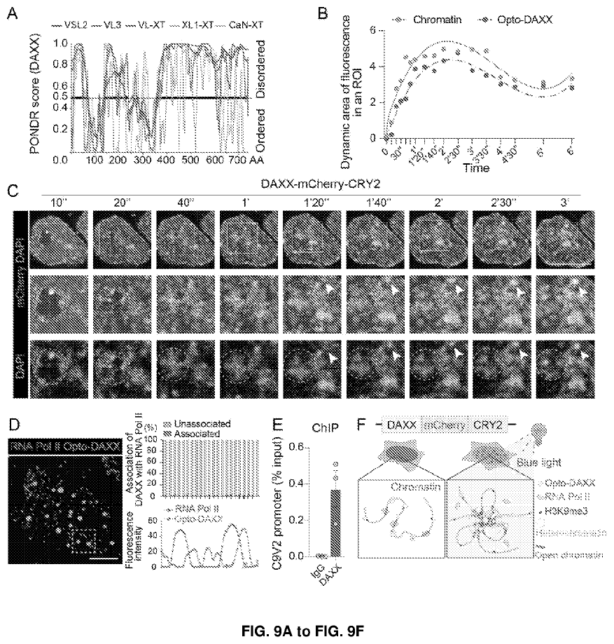

In accordance with the granular structures formed by DAXX in the cells, observations indicated that DAXX contains an extended intrinsically disordered region at its C-terminal half (FIG. 9A), which may provide its ability to undergo liquid-liquid phase separation and form granules via molecular condensation. To visualize the phase separation of DAXX in live cells, an “Opto-DAXX” protein model was developed by fusing DAXX with mCherry and a CRY2 domain, the latter being the photolyase homology region (PHR) of Arabidopsis thalana that self-clusters upon blue-light illumination (Shin et al., 2017; Taslimi et al., 2014). In contrast to the control mCherry-CRY2 protein, which remained diffuse in the cytoplasm without detectable changes in its distribution, DAXX-mCherry-CRY2 showed an exclusively nuclear distribution after exposure to blue light and formed abundant liquid droplets in a time-dependent manner when activated by the blue light (FIG. 2A). These DAXX droplets were formed through either separation from a dispersed phase or fusion of existing droplets (FIG. 2A, B). While a subset of the DAXX droplets were static, others showed dynamic behaviors of fusion and fission (FIG. 2A, B). Next, the optogenetic system was used to study the functional consequences of DAXX condensation in live cells. Using time-lapse photography, the dynamic changes in Opto-DAXX and chromatin signals were recorded during the light-induced phase separation of Opto-DAXX. The condensation of Opto-DAXX profoundly changed the shape and intensity of chromatin as visualized by DAPI staining (FIG. 9B, C), suggesting that DAXX phase separation restructures the chromatin conformation.

Next, changes in chromatin accessibility occurring as a result of DAXX phase separation were visualized using a super-resolution chromatin-imaging technology: the assay for transposase-accessible chromatin-photoactivated localization microscopy (ATAC-PALM), which visualizes the entire accessible genome on a nanometer scale through the use of a DNA probe comprising Tn5 transposase conjugated to bright photoactivatable Janelia Fluor 549 (Tn5-JF549) (FIG. 2C) (Cai et al., 2019: Xie et al., 2020). In the absence of blue light, Opto-DAXX distribution was diffuse and the Tn5-JF549 probe was scattered evenly across the nuclei (FIG. 2D). When illuminated by blue light, however, DAXX became condensed into granules, and the Tn5-JF549 probe assembled onto these granules with a pattern of significant colocalization (FIG. 2D, E). These data indicate that DAXX associated with accessible chromatins, consistent with an intrinsic role for DAXX in regulating gene expression; the data also indicate that the DAXX condensation is a strong regulator of chromatin structure.

Next, experiments were conducted to understand the effects of DAXX phase separation on genome topology. These experiments employed HiChIP, a technology that combines in situ high-throughput chromosome conformation capture (Hi-C) with chromatin immunoprecipitation (ChIP) (Mumbach et al., 2016), to profile three-dimensional chromatin architectures. IIEK293 cells expressing Opto-DAXX were exposed to blue-light illumination to induce the DAXX phase separation, and then the cells were subjected to in situ Hi-C contact generation and ChIP analysis using an antibody against DAXX. The same cells without blue-light illumination were used as a control. HiChTP sequencing results indicated that the DAXX condensation had increased the long-range chromatin interactions among the regulatory regions, such as enhancers and promoters, throughout the genome, whereas the interactions associated with the gene bodies were unaffected (FIG. 2G). The enhanced long-range interactions among the regulatory regions indicated that DAXX condensation may alter gene expression by modulating the chromatin spatial architecture.

Having established the function of DAXX as an anchor for the interactions of regulatory sequences, experiments were conducted to examine the transcription activity associated with the DAXX condensates. These experiments first tested the co-localization of RNA Pol II with the DAXX condensates in Opto-DAXX-expressing HEK293 cells after exposure to the blue-light illumination. Immunostaining analysis demonstrated that endogenous RNA Pol II was excluded from the Opto-DAXX droplets (FIG. 9D), indicating low levels of transcription activity at these sites. Furthermore, by using 5-ethynyl uridine and click chemistry to visualize newly synthesized RNAs, the signals for nascent RNAs were significantly decreased in the Opto-DAXX droplets when compared to those in other nuclear areas (FIG. 2F). Together, these data indicated that the phase separation of DAXX drives the assembly of distant open chromatins enriched in regulatory sequences and inhibits transcription of these genomic regions (FIG. 9F).

The increase in nuclear DAXX condensates in C9HRE patient cells and the ability of DAXX condensates to modulate chromatin structures indicated that the cells may have comprised a chromatin abnormality. To obtain a comprehensive understanding of the chromatin status in the C9HRE cells, an assay for transposase-accessible chromatin with high-throughput sequencing (ATAC-seq) was performed using iMNs from C9HRE patients and healthy individuals. By combining the ATAC-seq data with data provided by the NEUROLINCS database (Fraenkel, 2017), an ATAC-seq dataset derived from iMNs of ten C9HRE patients and six controls was compiled. Compared to the control iMNs, the C9HRE iMNs generally showed lower ATAC-seq peak signals, indicating a more compact genomic state under these conditions (FIG. 211). Since the chromatin accessibility of transcription start sites (TSSs) is essential for gene expression, the ATAC-seq data around TSSs was analyzed in more detail, which indicated that the chromatin accessibility at TSSs in the genomes of C9HRE patient iMNs was lower than that in control iMNs (FIG. 2I).

### Example 3—an Increase in DAXX Condensates Alters Epigenetic Regulations in C9HRE Patient Cells

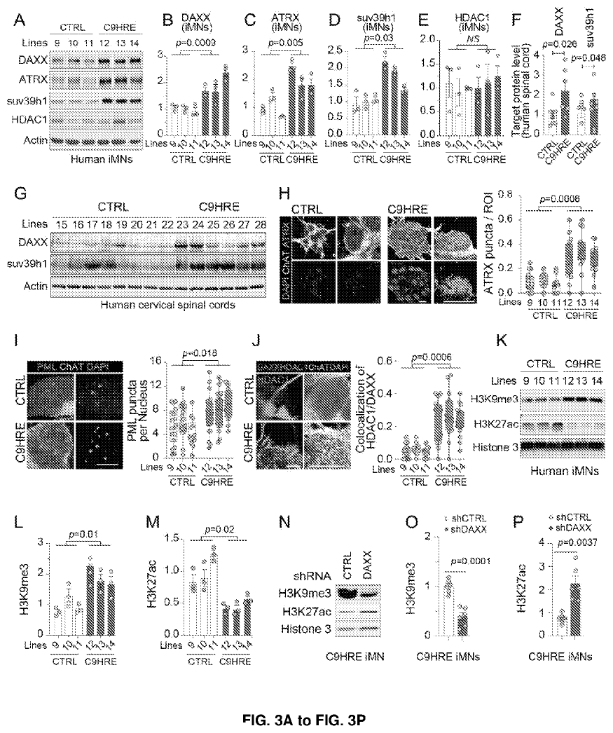

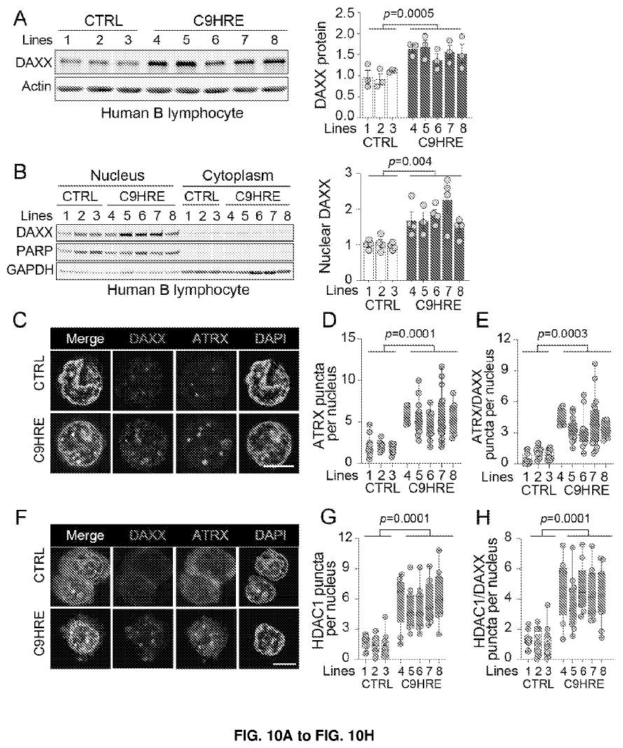

Phase separation of DAXX was contemplated to be influenced by concentration of the DAXX protein. In accordance with the increase in DAXX condensates in the C9HRE ALS cells (FIG. 1B, C), data indicated that the protein levels of DAXX on immunoblots were consistently higher in the C9HRE patient iMNs (FIG. 3A, B), spinal cords (FIG. 3F,G), and B lymphocytes (FIG. 10A) than in the control samples. Subcellular nucleocytoplasmic fractionation analysis of the B lymphocytes confirmed the nuclear accumulation of DAXX in the patient cells (FIG. 10B), consistent with the increase observed in the nuclear DAXX condensates (FIG. 1B, C).

Histone post-translational modifications are important epigenetic markers that induce changes in chromatin structure and transcriptional regulation. DAXX has been reported to form a complex with the transcriptional regulator ATRX and then recruit the histone lysine methyltransferase SUV9H1 to methylate H3K9 into H3K9me3 (He et al., 2015). Along with the increase in nuclear DAXX condensates shown by immunoblotting analysis, observations indicated that the levels of ATRX in patient-derived iMNs were higher than those in control iMNs (FIG. 3A, C). Moreover, SUV9H1 levels were consistently higher in the C9HRE ALS patient iMNs (FIG. 3A, D) and spinal cords (FIG. 3F, G) than those of the controls. Immunostaining analysis indicated that the quantity and size of the ATRX granules were significantly increased in the nuclei of the patient iMNs when compared to those in control iMNs (FIG. 3H). Similarly, C9HRE B lymphocytes showed more ATRX granules than did the controls; interestingly, a subset of the ATRX granules perfectly co-localized with the DAXX granules, suggesting that ATRX co-condensed with DAXX in the granules (FIG. 10C-E). The PML nuclear bodies (PML-NB) serve as a scaffold where DAXX and ATRX shuttle in and out to modify chromatins (Lallemand-Breitenbach and de Th6, 2018), and data indicated that the number of PML-NBs was significantly higher in the C9HRE patient iMNs than in control iMNs (FIG. 3I). In addition, DAXX was reported to inhibit the acetylation of H3K27 via the histone deacetylase HDAC1 (Li et al., 2000; Martire et al., 2019). Although the total levels of HDAC1 were unchanged in the patient cells, a significant increase in the nuclear localization of HDAC1 was observed, which showed a significant co-localization with DAXX condensates in the C9HRE iMNs (FIG. 3J) and B lymphocytes (FIG. 10F-H) when compared to that in the control cells, suggesting that DAXX phase separation could alter IIDAC1 activity at specific genomic regions in the patient cell lines.

One of the main functions of DAXX in the nucleus is related to histone modifications, specifically promoting H3K9me3 and repressing H3K27ac to regulate gene expression (He et al., 2015; Martire et al., 2019). Given the observation that DAXX was increased in C9HRE patient cells (FIG. 3A, B; FIG. 10A), especially in the nuclei (FIG. 1B, C; FIG. 10B), experiments were conducted to examine the global levels of H3K9me3 and H3K27ac in the C9HRE patient iMNs by immunoblotting. The data indicated that the C9HRE iMNs displayed significantly higher levels of H3K9me3 and lower levels of H3K27ac than did the control iMNs (FIG. 3K-M). Importantly, knockdown of DAXX rebalanced the global dysregulation of H3K9me3 and H3K27ac in the patient iMNs (FIG. 3N-P), demonstrating that DAXX plays a critical role in the dysregulation of the epigenetic histone markers in C9HRE ALS cells.

### Example 4—DAXX Suppresses Chromatin Accessibility at the C9Orf72 Locus in Patient Cells

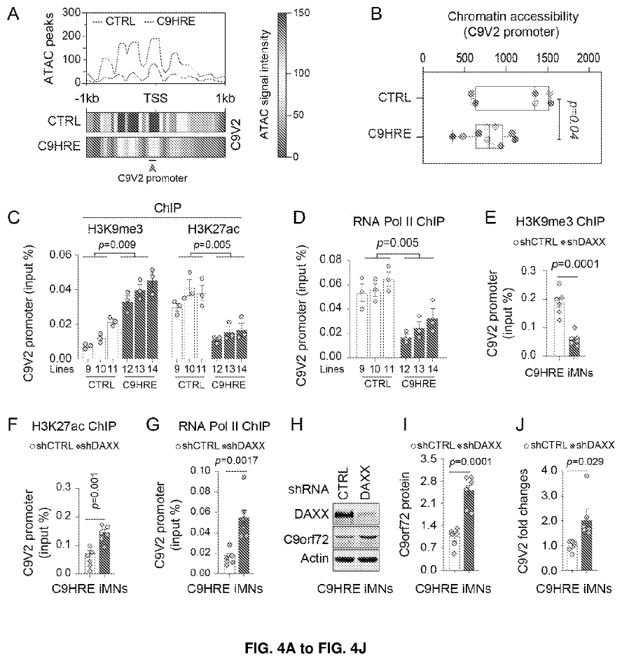

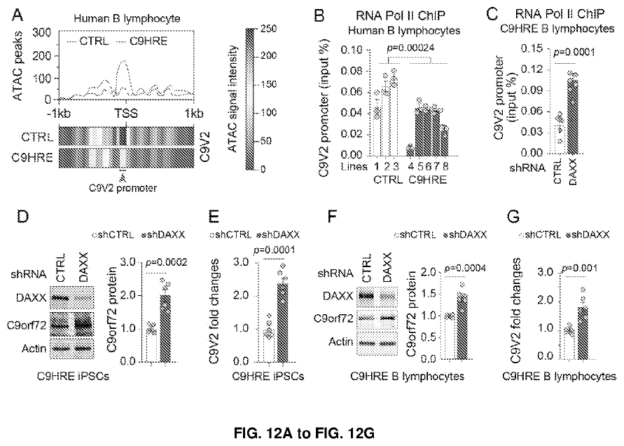

During the development of embodiments of the technology provided herein, experiments were conducted to collect additional data to provide information about DAXX-mediated regulation of gene expression. These experiments focused on the C9orf72 gene, which comprises the HRE mutation that directly associates with DAXX. The C9orf72 gene uses alternative start sites and splicing to produce at least three transcriptional variants called V1 (NM_145005.5), V2 (NM_018325.3), and V3 (NM_001256054.1), the sequences thereof being incorporated herein by reference. The HRE mutation is located either in the intron 1 region of transcripts V1 and V3 or in the putative promoter region of the transcript V2 upstream of its TSS (FIG. 11A). Consistent with previous reports (Balendra and Isaacs, 2018; Tran et al., 2015), V2 was the predominant transcript among the three variants, accounting for 80˜90% of the C9orf72 transcripts (FIG. 11B. C). The Eukaryotic Promoter Database (Dreos et al., 2014) was used to identify two GC box sites within 5 kb upstream of the V2 TSS (FIG. 11D). The first GC box, a 10-bp segment 257 bp upstream of the V2 TSS, is located within exon la of the V1/V3 transcript; the second GC box, a 49-bp segment immediately upstream of the V2 TSS, provides the entire intron 1b of the V1/V3 transcript (FIG. 11D). Given the second GC box's significant length and proximity to the TSS, it was contemplated that this region provides the promoter function for the V2 transcript. Indeed, when intron 1b was fused with an EGFP-coding sequence, intron 1b drove a robust expression of EGFP protein and mRNA (FIG. 11E-G), confirming the promoter activity of the region, hereafter termed the C9V2 promoter. The (G4C2)ₙ repeat is located immediately upstream of the C9V2 promoter (FIG. 11D), and expansion of the repeats could disrupt V2 expression. Analyzing the ATAC-seq dataset derived from iMNs of C9HRE patients and controls indicated that the patient iMNs had a significant reduction in chromatin accessibility at the C9V2 promoter region when compared to that of the control iMNs, (FIG. 4A, B). Consistently, B lymphocytes harboring the C9HRE mutation exhibited a similar loss of chromatin accessibility at the C9orf72 promoter region (FIG. 12A). Since V2 is the predominant variant among C9orf72 transcripts, these data indicate that a reduction in the V2 transcript underlies the loss of C9orf72 expression in patient cells with HRE mutations (DeJesus-Hernandez et al., 2011; van Blitterswijk et al., 2015).

During the development of embodiments of the technology provided herein, experiments were conducted to understand the epigenetic mechanism through which the expansion of the (G4C2)₁ repeats influences V2 expression. These experiments examined the occupancy of H3K9me3 or H3K27ac, the markers for transcription suppression or activation, respectively, at the V2 promoter in C9HRE patient iMNs. Using ChIP-qPCR with antibodies specifically directed against H3K9me3 or H3K27ac, the data indicated a profound increase in H3K9me3 occupancy as well as a substantial decrease in H3K27ac occupancy at the V2 promoter in the C9HRE patient iMNs as compared to those in control iMNs (FIG. 4C), suggesting reduced transcription activity at the promoter region containing the HRE mutation. Furthermore, ChIP-qPCR analysis using antibodies against RNA polymerase II showed a significant reduction in the polymerase occupancy at the V2 promoter in the C9HRE patient iMNs when compared to that in the control iMNs (FIG. 4D), confirming the repressed transcription status of this region containing the HRE mutation. In addition, B lymphocytes from C9HRE patients also showed a much lower occupancy of RNA polymerase II at the V2 promoter than did control cells (FIG. 12B).

Next, the effects of DAXX knockdown on epigenetic and transcriptional changes in C9HRE patient iMNs were evaluated to understand the role of DAXX in HRE-dependent genomic regulation. A partial loss of DAXX induced by shRNAs significantly reduced the occupancy of H3K9me3 at the C9V2 promoter but increased the occupancy of H3K27ac at this site, as indicated by ChIP-qPCR analysis (FIG. 4E, F). Furthermore, DAXX knockdown led to a significant increase in the recruitment of RNA polymerase II to the C9orf72 V2 promoter in the C9HRE iMNs (FIG. 4G) and B lymphocytes (FIG. 12C). Accordingly, these data indicated that DAXX knockdown significantly increased the expression of both C9orf72 protein and V2 mRNA in the C9HRE iMNs (FIG. 4H-J); and in iPSCs (FIG. 12D, E) B lymphocytes (FIG. 12F, G) carrying the HRE mutations. These results collectively indicate that the HRE mutations alter the epigenetic modifications and reduce the chromatin accessibility of the locus, thereby repressing the expression of the C9orf72 gene and demonstrate that DAXX plays a critical role in mediating HRE-dependent transcriptional suppression.

### Example 5—Stress-Dependent Induction of C9Orf72 Expression is Lost in HRE-Harboring Patient Cells

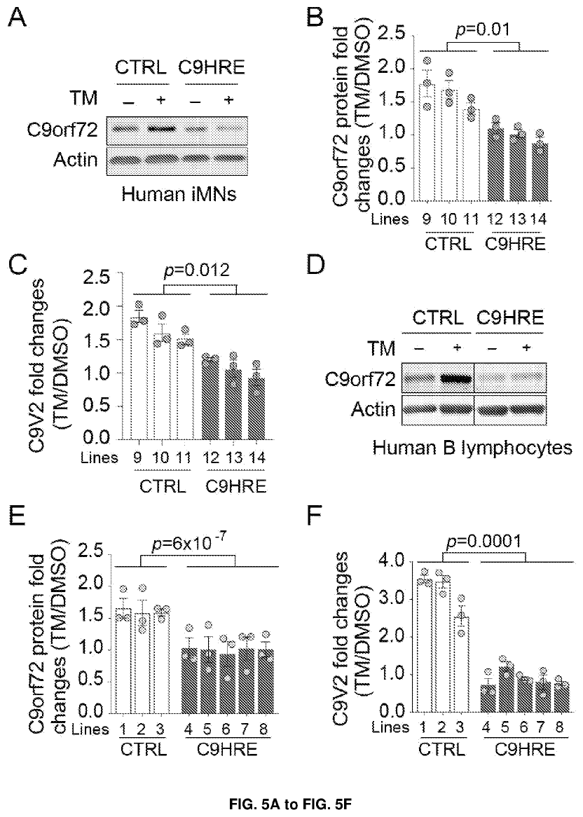

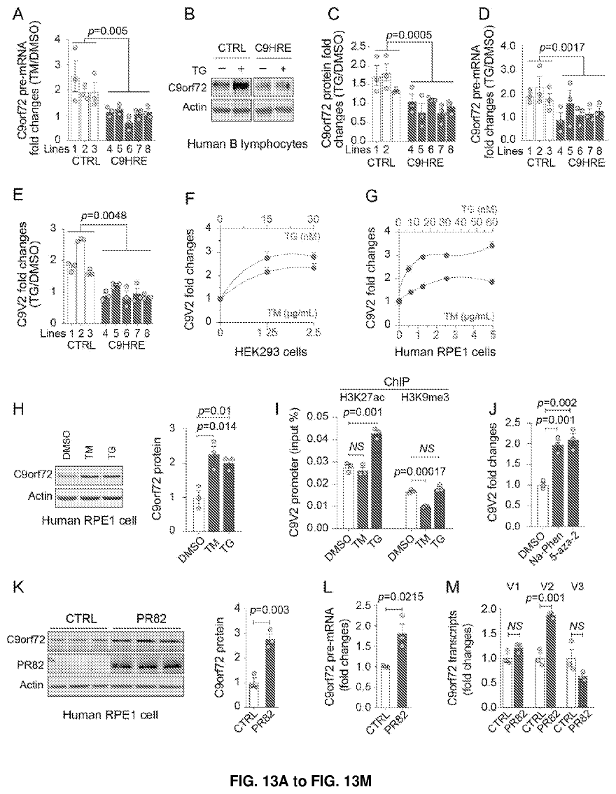

Data collected while testing the transcriptional regulation of C9orf72 indicated that the expression of C9orf72 was enhanced by stress in normal cells but this dynamic regulation was lost in patient cells harboring the HRE mutations. Accordingly, experiments were conducted to assess the transcriptional regulation of C9orf72 under experimental conditions of neurodegeneration-associated proteotoxic stress, such as that induced by tunicamycin, which inhibits protein N-glycosylation to disrupt ER-associated degradation (Cherepanova et al., 2016), or thapsigargin, which induces ER stress by selectively inhibiting ER Ca+-ATPases (Lytton et al., 1991). When iMNs from healthy individuals were stressed by exposure to tunicamycin, both C9orf72 protein and V2 mRNA levels were found to be significantly increased (FIG. 5A-C). In contrast, such tunicamycin-induced upregulation of C9orf72 expression was absent from iMNs derived from C9HRE patients (FIG. 5A-C). Similarly, the stress-responsive induction of C9orf72 expression at both the protein and mRNA levels was observed in normal B lymphocytes treated with either tunicamycin or thapsigargin, but the dynamic regulation of C9orf72 expression was lost in multiple lines of B lymphocytes from C9HRE patients (FIG. 5D-F; FIG. 13A-E).

The stress-responsive induction of the C9orf72 V2 transcript and the corresponding protein was also observed in other cell types, such as human RPE1 cells (FIG. 13G, H) and HEK293 cells (FIG. 13F). Given the importance of C9orf72 in physiological and disease-associated processes, the mechanisms through which C9orf72 transcription is regulated by stress were next investigated. Experiments were conducted to evaluate how stressors modulated epigenetic modifications at the C9orf72 V2 promoter and the data indicated that thapsigargin treatment decreased the levels of the repressive marker H3K9me3, whereas tunicamycin treatment elevated the levels of the active marker H3K27ac at the locus in human RPE1 cells (FIG. 13I), suggesting that both epigenetic mechanisms are involved in the stress-dependent induction of C9orf72. Indeed, treating RPE1 cells with the methyltransferase inhibitor 5-aza-2′-deoxycytidine (5-aza-2; also known as 5-AZA-CdR, decitabine) to suppress histone methylation (Nguyen et al., 2002) or with the histone deacetylase inhibitor sodium phenylbutyrate (Na-Phen) to enhance histone acetylation (Warrell et al., 1998) significantly upregulated the levels of C9orf72 V2 transcripts (FIG. 13J). Taken together, these data suggest that stress-dependent transcriptional regulation is mediated by the epigenetic modification of the C9orf72 V2 promoter.

It was further observed that expression of C9orf72 was induced by stress from toxic proteins associated with neurodegenerative diseases. Proline-arginine (PR) poly-dipeptides are one type of proteotoxic products translated from expanded G4C2 repeat RNAs. Expressing a construct with randomized codons encoding 82 repeats of poly-PR dipeptides (PR82) in RPE1 cells significantly increased the levels of the C9orf72 pre-mRNA and protein (FIG. 13K, L). Notably, only the C9orf72 V2 transcript, and not the V1 or V3 transcript, was significantly changed, indicating that the stress-dependent regulation of C9orf72 expression is specific to the V2 transcript (FIG. 13M).

### Example 6—DAXX Mediates HRE-Dependent Inhibition of Stress-Induced Transcription of C9Orf72

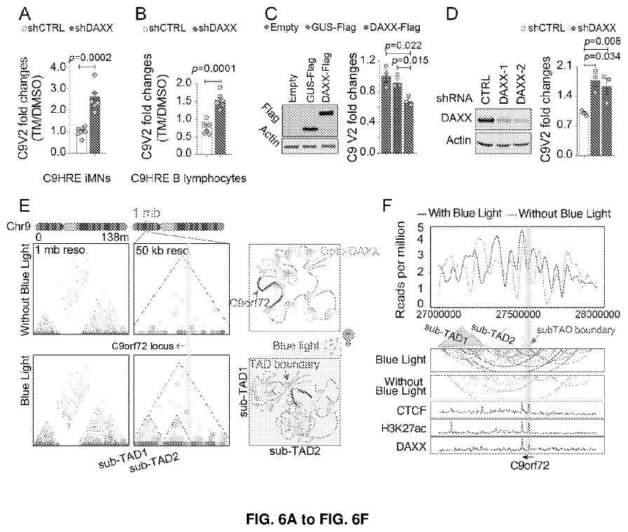

DAXX accumulates in the nuclei of cells harboring the C9orf72 HRE mutation and functions in epigenetic regulation. Accordingly, experiments were conducted during the development of embodiments of the technology described herein to test whether DAXX mediates the inhibition of stress-induced C9orf72 transcription in patient cells. In particular, RT-qPCR analysis was used to quantify the stress-induced expression of the C9orf72 V2 transcript in patient cells after knockdown of DAXX. Data collected indicated that knockdown of DAXX in C9HRE iMNs restored the tunicamycin-induced upregulation of the V2 mRNA levels, which did not occur in the control shRNA-treated C9HRE iMNs (FIG. 6A). Moreover, DAXX knockdown had a similar effect on the C9HRE B lymphocytes (FIG. 6B). Together, these data demonstrate that DAXX mediates HRE-dependent inhibition of the stress-induced transcription of C9orf72.

The diseases linked to the C9orf72 HRE mutations are dominantly inherited, and most patient cells carry one expanded repeat allele and one wild-type allele. Since the patient cells exhibited a complete loss of stress-induced C9orf72 expression, experiments were conducted to investigate the mechanism through which stress-induced transcription at the wild-type allele was suppressed in the patient cells. Since HRE mutations induce the accumulation of DAXX in the nuclei of patient cells, experiments were conducted to test if elevated levels of DAXX suppress C9orf72 expression at the wild-type allele. Since the C9orf72 V2 transcript specifically underlies the stress-induced expression of the gene (FIG. 5; FIG. 13A-M), the potential association of DAXX with the V2 promoter in wild-type cells was tested. ChIP-qPCR analysis in human RPE1 cells showed that significantly more V2 promoter DNA was pulled down by an antibody against endogenous DAXX than by an IgG control (FIG. 9E), demonstrating the intrinsic occupancy of DAXX on the V2 promoter at the wild-type allele. To test the effects of elevated levels of DAXX on C9orf72 expression, DAXX was over-expressed in wild-type cells and observed a significant reduction in C9orf72 V2 transcripts as compared with the empty vector or a GUS reference protein control (FIG. 6C). The role of DAXX in negatively regulating C9or172 transcription was further confirmed by the increase in V2 transcript levels upon DAXX knockdown in wild-type cells (FIG. 6D). Together, these data support the notion that the nuclear accumulation of DAXX as a result of the HRE mutations in patient cells can lead to a suppression of V2 transcription from the wild-type allele.

Next, experiments were conducted to understand better the mechanistic role of DAXX phase separation in the regulation of C9orf72 transcription. HiChIP-seq analysis was used to examine the 3D chromatin structural shifts at the C9orf72 locus as a result of the inducible phase separation of DAXX. Consistent with the genome-wide observation data, the condensation of Opto-DAXX promoted 3D chromatin interactions across chromosome 9, as indicated by the enhanced signals in the interaction matrix plots (FIG. GE). Topologically associated domains (TAD) are the architectural units of 3D chromatins, in which there is a high frequency of chromatin interactions (Dixon et al., 2012). With a 50-kb resolution for the TAD analysis, it was found that the phase separation of Opto-DAXX led to the formation of two new sub-TADs (FIG. GE, F). TAD boundaries are frequently located in and around TSS that are enriched with CTCF, a chromatin insulator involved in boundary establishment (Dixon et al., 2012). Disruption of topological boundaries can cause dysregulation of gene expression at the boundaries (Sun et al., 2018). The TAD analysis revealed a new boundary formed at the C9orf72 promoter region as a result of Opto-DAXX condensation (FIG. GE, F). An analysis of previously reported ChIP-seq data indicated that the peaks of CTCF and DAXX overlap at the site of the newly formed sub-TAD boundary at the C9orf72 promoter (FIG. GE, F), indicating that both CTCF and DAXX participated in the establishment of the boundary. Furthermore, an II3K27ac peak also co-localized with the CTCF and DAXX peaks and the newly formed TAD boundary at the C9orf72 promoter locus (FIG. GF), indicating that both chromatin conformation and histone modification contribute to the DAXX-dependent regulation of transcription activity at the C9orf72 locus. Thus, these data together suggest that DAXX acts as a key regulator of transcription at the C9orf72 locus through its modulation of 3D chromatin interactions.

### Example 7—DAXX Regulates the Susceptibility of C9Orf72 HRE Motor Neurons to Stress

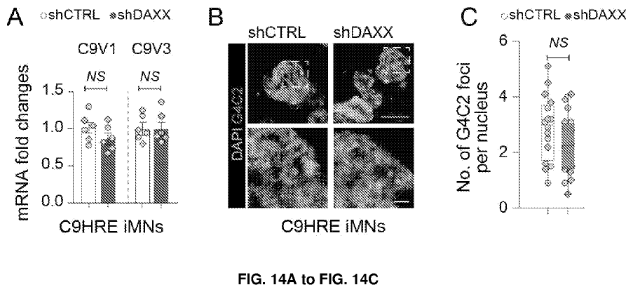

Data collected during the experiments described herein indicated that HRE-dependent accumulation of DAXX suppresses the expression of C9orf72 by acting on the promoter of the predominant V2 transcript. Unlike V2, which has the hexanucleotide repeats in its promoter (FIG. 11A), the C9orf72 V1 and V3 transcripts contain the repeats in their first intron and thus are responsible for the disease-associated RNA toxicity and RAN translation, both of which originate from the repeat-containing V1 and V3 transcripts. Experiments indicated that knockdown of DAXX did not significantly affect the levels of V1 or V3 transcripts, as shown by RT-qPCR analysis of these transcripts in C9HRE iMNs with or without DAXX knockdown (FIG. 14A). RNA FISH was also used to directly detect the G4C2 repeat RNA foci, which provide a marker for repeat RNAs in C9HRE iMNs (Sareen et al., 2013). With the loss of DAXX, there was no significant change in the quantity or size of the RNA foci, as indicated by the FISH analysis (FIG. 14B, C). These results indicate that DAXX regulates the expression of C9orf72 primarily through its action on the V2 promoter and has little effect on the transcription of the repeat-containing V1 or V3 transcripts.

Motor neurons differentiated from iPSCs carrying the C9orf72 HRE mutation are susceptible to the stress induced by tunicamycin (Haeusler et al., 2014). Given the function of DAXX in regulating the expression of C9orf72 under stress, experiments were conducted to investigate the role of DAXX in mediating the sensitivity of C9HRE iMNs. Whereas tunicamycin induced a time-dependent loss of C9HRE iMNs, a partial knockdown of DAXX led to significantly higher survival rates for the C9HRE iMNs than for the controls (FIG. 7A, B), indicating that reducing DAXX levels has a neuroprotective effect in C9HRE patient cells.

Next, experiments were conducted to assess the effects of modulating DAXX-dependent epigenetic markers on the sensitivity of C9HRE iMNs to tunicamycin-induced stress. The HRE-associated accumulation of DAXX led to increased levels of II3K9me3 and decreased levels of II3K27ac in the patient cells. To rebalance the increased levels of H3K9me3, the C9HRE iMNs were treated with 5-aza-2, which reverses the methylation of H3K9 (Nguyen et al., 2002), and the demethylation treatment led to a significant increase in the survival of C9HRE iMNs exposed to tunicamycin (FIG. 7C, D). Furthermore, to reverse the decrease in the levels of H3K27ac, the C9HRE iMNs were treated with Na-Phen, the HDAC inhibitor that increases the acetylation of H3K27 (Warrell et al., 1998); this pro-acetylation treatment yielded robust protection of the C9HRE iMNs from tunicamycin-induced stress (FIG. 7C, D). Taken together, these data suggest that both DAXX itself and the associated downstream epigenetic events can be modulated to alleviate the sensitivity of C9orf72 HRE motor neurons to stress.

All publications and patents mentioned in the above specification and listed below in the REFERENCES section are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

### REFERENCES

Amick, J., Roczniak-Ferguson, A., and Ferguson, S. M. (2016). C9orf72 binds SMCR8, localizes to lysosomes and regulates mTORC1 signaling. Mol Biol Cell 27. 3040-3051 Appleby-Mallinder, C., Schaber, E., Kirby, J., Shaw, P. J., Cooper-Knock, J., Heath, P. R., and Highley, J. R. (2021). TDP43 proteinopathy is associated with aberrant DNA methylation in human amyotrophic lateral sclerosis. Neuropathol Appl Neurobiol 47, 61-72.
Ash, P. E., Bieniek, K. F., Gendron, T. F., Caulfield, T., Lin, W. L., Dejesus-Hernandez, M., van Blitterswijk, M. M., Jansen-West, K., Paul, J. W., 3rd, Rademakers, R., et al. (2013). Unconventional Translation of C9ORF72 GGGGCC Expansion Generates Insoluble Polypeptides Specific to c9FTD/ALS. Neuron 77, 639-646.
Balendra, R., and Isaacs, A. M. (2018). C9orf72-mediated ALS and FTD: multiple pathways to disease. Nature Reviews Neurology 14, 544-558.
Barbosa, L. F., Cerqueira, F. M., Macedo, A. F., Garcia, C. C., Angeli, J. P., Schumacher, R. I., Sogayar, M. C., Augusto, O., Carri, M. T., Di Mascio, P., and Medeiros, M. H. (2010). Increased SOD1 association with chromatin, DNA damage, p53 activation, and apoptosis in a cellular model of SOD1-linked ALS. Biochim Biophys Acta 1802, 462-471.
Batut, B., Hiltemann, S., Bagnacani, A., Baker, D., Bhardwaj, V., Blank, C., Bretaudeau, A., Brillet-Guéguen, L., Čech, M., Chilton, J., et al. (2018). Community-Driven Data Analysis Training for Biology. Cell Syst 6, 752-758.
Belzil, V. V., Bauer, P. O., Prudencio, M., Gendron, T. F., Stetler, C. T., Yan, I. K., Pregent, L., Daughrity, L., Baker, M. C., Rademakers, R., et al. (2013). Reduced C9orf72 gene expression in c9FTD/ALS is caused by histone trimethylation, an epigenetic event detectable in blood. Acta Neuropathol 126, 895-905.
Berson, A., Nativio, E., Berger, S. L., and Bonini, N. M. (2018). Epigenetic Regulation in Neurodegenerative Diseases. Trends Neurosci 41, 587-598.
Berson, A., Sartoris, A., Nativio, R., Van Deerlin, V., Toledo, J. B., Porta, S., Liu, S., Chung, C. Y., Garcia, B. A., Lee, V. M., et al. (2017). TDP-43 Promotes Neurodegeneration by Impairing Chromatin Remodeling. Curr Biol 27, 3579-3590.
Bieniek, K. F., van Blitterswijk, M., Baker, M. C., Petrucelli, L., Rademakers, R., and Dickson, D. W. (2014). Expanded C9ORF72 Hexanucleotide Repeat in Depressive Pseudodementia. JAMA Neurology 71, 775-781.
Buenrostro, J. D., Wu, B., Chang, H. Y., and Greenleaf, W. J. (2015). ATAC-seq: A Method for Assaying Chromatin Accessibility Genome-Wide. Curr Protoc Mol Biol 109, 21.29.1-21.29.9
Burberry, A., Suzuki, N., Wang, J. Y., Moccia, R., Mordes, D. A., Stewart, M. H., Suzuki-Uematsu, S., Ghosh, S., Singh, A., Merkle, F. T., et al. (2016). Loss-of-function mutations in the C9ORF72 mouse ortholog cause fatal autoimmune disease. Science translational medicine 8, 347ra393.
Cai, D., Feliciano, D., Dong, P., Flores, E., Gruebele, M., Porat-Shliom, N., Sukenik, S., Liu, Z., and Lippincott-Schwartz, J. (2019). Phase separation of YAP reorganizes genome topology for long-term YAP target gene expression. Nat Cell Biol 21, 1578-1589.
Campeau, E., Ruhl, V. E., Rodier, F., Smith, C. L., Rahmberg, B. L., Fuss, J. O., Campisi, J., Yaswen, P., Cooper, P. K., and Kaufman, P. D. (2009). A versatile viral system for expression and depletion of proteins in mammalian cells. PLoS One 4, e6529.
Chaumeil, J., Micsinai, M., and Skok, J. A. (2013). Combined immunofluorescence and DNA FISII on 3D-preserved interphase nuclei to study changes in 3D nuclear organization. J Vis Exp 3, e50087.
Cherepanova, N., Shrimal, S., and Gilmore, R. (2016). N-linked glycosylation and homeostasis of the endoplasmic reticulum. Current Opinion in Cell Biology 41, 57-65.
Consortium, E. P. (2004). The ENCODE (ENCyclopedia Of DNA Elements) Project. Science 306, 636-640.
DeJesus-Hernandez, M., Mackenzie, Ian R., Boeve, Bradley F., Boxer, Adam L., Baker, M., Rutherford, Nicola J., Nicholson, Alexandra M., Finch, NiCole A., Flynn, H., Adamson, J., et al. (2011). Expanded GGGGCC Hexanucleotide Repeat in Noncoding Region of C9ORF72 Causes Chromosome 9p-Linked FTD and ALS. Neuron 72, 245-256.
Dixon, J. R., Selvaraj, S., Yue, F., Kim, A., Li, Y., Shen, Y., Hu, M., Liu, J. S., and Ren, B. (2012). Topological domains in mammalian genomes identified by analysis of chromatin interactions. Nature 485, 376-380.
Donnelly, C. J., Zhang, P. W., Pham, J. T., Heusler, A. R., Mistry, N. A., Vidensky, S., Daley, E. L., Poth, E. M., Hoover, B., Fines, D. M., et al. (2013). RNA toxicity from the ALS/FTD C9ORF72 expansion is mitigated by antisense intervention. Neuron 80, 415-428.
Dreos, R., Ambrosini, G., Périer, R. C., and Bucher, P. (2014). The Eukaryotic Promoter Database: expansion of EPDnew and new promoter analysis tools. Nucleic Acids Research 43, D92-D96.
Fasciani, A., D'Annunzio, S., Poli, V., Fagnocchi, L., Beyes, S., Michelatti, D., Corazza, F., Antonelli, L., Gregoretti, F., Oliva, G., et al. (2020). MLL4-associated condensates counterbalance Polycomb-mediated nuclear mechanical stress in Kabuki syndrome. Nat Genet 52, 1397-1411.
Felix Krueger, F. J., Phil Ewels, Ebrahim Afyounian, & Benjamin Schuster-Boeckler (2021). FelixKrueger/TrimGalore: v0.6.7. Zenodo
Fraenkel, -. E. (2017).—diMN (Exp 3)—ALS and Control (unaffected) diMN cell lines differentiated from iPS cell lines using a short and direct differeintiation protocol -ATAC-seq. http://identifiers.org/lines.data/LDG-1394.
Gendron, T. F., Chew, J., Stankowski, J. N., Hayes, L. R., Zhang, Y. J., Prudencio, M., Carlomagno, Y., Daughrity, L. M., Jansen-West, K., Perkerson, E. A., et al. (2017). Poly(GP) proteins are a useful pharmacodynamic marker for C9ORF72-associated amyotrophic lateral sclerosis. Science translational medicine 9, eaai7866.
Goldman, J. S., Quinzii, C., Dunning-Broadbent, J., Waters, C., Mitsumoto, H., Brannagan, T. H., III, Cosentino, S., Huey, E. D., Nagy, P., and Kuo, S.-H. (2014). Multiple System Atrophy and Amyotrophic Lateral Sclerosis in a Family With Hexanucleotide Repeat Expansions in C9orf72. JAMA Neurology 71, 771-774.
Haeusler, A. R., Donnelly, C. J., Periz, G., Simko, E. A., Shaw, P. G., Kim, M. S., Maragakis, N.J., Troncoso, J. C., Pandey, A., Sattler, R., et al. (2014). C9orf72 nucleotide repeat structures initiate molecular cascades of disease. Nature 507, 195-200.
He, Q., Kim, H., Huang, R., Lu, W., Tang, M., Shi, F., Yang, D., Zhang, X., Huang, J., Liu, D., and Songyang, Z. (2015). The Daxx/Atrx Complex Protects Tandem Repetitive Elements during DNA Hypomethylation by Promoting H3K9 Trimethylation. Cell Stem Cell 17, 273-286.
Hensman Moss, D. J., Poulter, M., Beck, J., Hehir, J., Polke, J. M., Campbell, T., Adamson, G., Mudanohwo, E., McColgan, P., Haworth, A., et al. (2014). C9orf72 expansions are the most common genetic cause of Huntington disease phenocopies. Neurology 82, 292-299.
Huo, X., Ji, L., Zhang, Y., Lv, P., Cao, X., Wang, Q., Yan, Z., Dong, S., Du, D., Zhang, F., et al. (2020). The Nuclear Matrix Protein SAFB Cooperates with Major Satellite RNAs to Stabilize Heterochromatin Architecture Partially through Phase Separation. Molecular Cell 77, 368-383.
Lallemand-Breitenbach, V., and de Th6, H. (2018). PML nuclear bodies: from architecture to function. Current opinion in cell biology 52, 154-161.
Langmead, B., and Salzberg, S. L. (2012). Fast gapped-read alignment with Bowtie 2. Nat Methods 9, 357-359.
Li, H., Leo, C., Zhu, J., Wu, X., O'Neil, J., Park, E.-J., and Chen, J. D. (2000). Sequestration and Inhibition of Daxx-Mediated Transcriptional Repression by PML. Molecular and Cellular Biology 20, 1784-1796.
Lin, D.-Y., Huang, Y.-S., Jeng, J.-C., Kuo, H.-Y., Chang, C.-C., Chao, T.-T., Ho, C.-C., Chen, Y.-C., Lin, T.-P., Fang, H.-I., et al. (2006). Role of SUMO-Interacting Motif in Daxx SUMO Modification, Subnuclear Localization, and Repression of Sumoylated Transcription Factors. Molecular Cell 24, 341-354.
Liu, E. Y., Russ, J., Wu, K., Neal, D., Suh, E., McNally, A. G., Irwin, D. J., Van Deerlin, V. M., and Lee, E. B. (2014). C9orf72 hypermethylation protects against repeat expansion-associated pathology in ALS/FTD. Acta Neuropathol 128, 525-541.
Liu, Y., Wang, T., Ji, Y. J., Johnson, K., Liu, H., Johnson, K., Bailey, S., Suk, Y., Lu, Y.-N., Liu, M., and Wang, J. (2018). A C9orf72-CARM1 axis regulates lipid metabolism under glucose starvation-induced nutrient stress. Genes & Development 32, 1380-1397.
Lytton, J., Westlin, M., and Hanley, M. R. (1991). Thapsigargin inhibits the sarcoplasmic or endoplasmic reticulum Ca-ATPase family of calcium pumps. Journal of Biological Chemistry 266, 17067-17071.
Majounie, E., Abramzon, Y., Renton, A. E., Perry, R., Bassett, S. S., Pletnikova, O., Troncoso, J. C., Hardy, J., Singleton, A. B., and Traynor, B. J. (2012). Repeat expansion in C9ORF72 in Alzheimer's disease. N Engl J Med 366, 283-284.
Malik, I., Kelley, C. P., Wang, E. T., and Todd, P. K. (2021). Molecular mechanisms underlying nucleotide repeat expansion disorders. Nat Rev Mol Cell Biol 22, 589-607.
Martire, S., Gogate, A. A., Whitmill, A., Tafessu, A., Nguyen, J., Teng, Y.-C., Tastemel, M., and Banaszynski, L. A. (2019). Phosphorylation of histone H3.3 at serine 31 promotes p300 activity and enhancer acetylation. Nature Genetics 51, 941-946.
Meisler, M. H., Grant, A. E., Jones, J. M., Lenk, G. M., He, F., Todd, P. K., Kamali, M., Albin, R. L., Lieberman, A. P., Langenecker, S. A., and McInnis, M. G. (2013). C9ORF72 expansion in a family with bipolar disorder. Bipolar Disord 15, 326-332.
Mizielinska, S., Gronke, S., Niccoli, T., Ridler, C. E., Clayton, E. L., Devoy, A., Moens, T., Norona, F. E., Woollacott, I. O., Pietrzyk, J., et al. (2014). C9orf72 repeat expansions cause neurodegeneration in Drosophila through arginine-rich proteins. Science 345, 1192-1194.
Mori, K., Weng, S. M., Arzberger, T., May, S., Rentzsch, K., Kremmer, E., Schmid, B., Kretzschmar, H. A., Cruts, M., Van Broeckhoven, C., et al. (2013). The C9orf72 GGGGCC repeat is translated into aggregating dipeptide-repeat proteins in FTLD/ALS. Science 339, 1335-1338.
Mumbach, M. R., Rubin, A. J., Flynn, R. A., Dai, C., Khavari, P. A., Greenleaf, W. J., and Chang, H. Y. (2016). HiChIP: efficient and sensitive analysis of protein-directed genome architecture. Nature Methods 13, 919-922.
Nguyen, C. T., Weisenberger, D. J., Velicescu, M., Gonzales, F. A., Lin, J. C., Liang, G., and Jones, P. A. (2002). Histone H3-lysine 9 methylation is associated with aberrant gene silencing in cancer cells and is rapidly reversed by 5-aza-2′-deoxycytidine. Cancer Res 62. 6456-6461.
O'Rourke, J. G., Bogdanik, L., Yanez, A., Lall, D., Wolf, A. J., Muhammad, A. K., Ho, R., Carmona, S., Vit, J. P., Zarrow, J., et al. (2016). C9orf72 is required for proper macrophage and microglial function in mice. Science 351, 1324-1329.
Paez-Colasante, X., Figueroa-Romero, C., Sakowski, S. A., Goutman, S. A., and Feldman, E. L. (2015). Amyotrophic lateral sclerosis: mechanisms and therapeutics in the epigenomic era. Nat Rev Neurol 11, 266-279.
Renton, Alan E., Majounie, E., Waite, A., Sim6n-Sinchez, J., Rollinson, S., Gibbs, J. R., Schymick, Jennifer C., Laaksovirta, II., van Swieten, John C., Myllykangas, L., et al. (2011). A Hexanucleotide Repeat Expansion in C9ORF72 Is the Cause of Chromosome 9p21-Linked ALS-FTD. Neuron 72, 257-268.
Ryu, H., Smith, K., Camelo, S. I., Carreras, I., Lee, J., Iglesias, A. H., Dangond, F., Cormier, K. A., Cudkowicz, M. E., H. Brown Jr, R., and Ferrante, R. J. (2005). Sodium phenylbutyrate prolongs survival and regulates expression of anti-apoptotic genes in transgenic amyotrophic lateral sclerosis mice. Journal of Neurochemistry 93, 1087-1098.
Sareen, D., O'Rourke, J. G., Meera, P., Muhammad, A. K. M. G., Grant, S., Simpkinson, M., Bell, S., Carmona, S., Ornelas, L., Sahabian, A., et al. (2013). Targeting RNA Foci in iPSC-Derived Motor Neurons from ALS Patients with a C9ORF72 Repeat Expansion. Science Translational Medicine 5, 208ra149.
Sellier, C., Campanari, M. L., Julie Corbier, C., Gaucherot, A., Kolb-Cheynel, I., Oulad-Abdelghani, M., Ruffenach, F., Page, A., Ciura, S., Kabashi, E., and Charlet-Berguerand, N. (2016). Loss of C9ORF72 impairs autophagy and synergizes with polyQ Ataxin-2 to induce motor neuron dysfunction and cell death. EMBO J 35, 1276-1297.
Servant, N., Lajoie, B. R., Nora, E. P., Giorgetti, L., Chen, C. J., Heard, E., Dekker, J., and Barillot, E. (2012). HiTC: exploration of high-throughput ‘C’ experiments. Bioinformatics 28, 2843-2844.
Servant, N., Varoquaux, N., Lajoie, B. R., Viara, E., Chen, C.-J., Vert, J.-P., Heard, E., Dekker, J., and Barillot, E. (2015). HiC-Pro: an optimized and flexible pipeline for Hi-C data processing. Genome Biology 16, 259.
Shi, Y., Lin, S., Staats, K. A., Li, Y., Chang, W. H., Hung, S. T., Hendricks, E., Linares, G. R., Wang, Y., Son, E. Y., et al. (2018). Haploinsufficiency leads to neurodegeneration in C9ORF72 ALS/FTD human induced motor neurons. Nat Med 24, 313-325.
Shin, Y., Berry, J., Pannucci, N., Haataja, M. P., Toetteher, J. E., and Brangwynne, C. P. (2017). Spatiotemporal Control of Intracellular Phase Transitions Using Light-Activated optoDroplets. Cell 168, 159-171.
Shin, Y., Chang, Y. C., Lee, D. S. W., Berry, J., Sanders, D. W., Ronceray, P., Wingreen, N. S., Haataja, M., and Brangwynne, C. P. (2018). Liquid Nuclear Condensates Mechanically Sense and Restructure the Genome. Cell 175, 1481-1491.
Sullivan, P. M., Zhou, X., Robins, A. M., Paushter, D. H., Kim, D., Smolka, M. B., and Hu, F. (2016). The ALS/FTLD associated protein C9orf72 associates with SMCR8 and WDR41 to regulate the autophagy-lysosome pathway. Acta Neuropathol Commun 4, 51.
Sun, J. H., Zhou, L., Emerson, D. J., Phyo, S. A., Titus, K. R., Gong, W., Gilgenast, T. G., Beagan, J. A., Davidson, B. L., Tassone, F., and Phillips-Cremins, J. E. (2018). Disease-Associated Short Tandem Repeats Co-localize with Chromatin Domain Boundaries. Cell 175, 224-238.
Tang, J., Qu, L. K., Zhang, J., Wang, W., Michaelson, J. S., Degenhardt, Y. Y., El-Deiry, W. S., and Yang, X. (2006). Critical role for Daxx in regulating Mdm2. Nat Cell Biol 8, 855-862.
Taslimi, A., Vrana, J. D., Chen, D., Borinskaya, S., Mayer, B. J., Kennedy, M. J., and Tucker, C. L. (2014). An optimized optogenetic clustering tool for probing protein interaction and function. Nature communications 5, 4925.
Tóth, G., Gáspári, Z., and Jurka, J. (2000). Microsatellites in different eukaryotic genomes: survey and analysis. Genome Res 10, 967-981.
Tran, H., Almeida, S., Moore, J., Gendron, Tania F., Chalasani, U., Lu, Y., Du, X., Nickerson, Jeffrey A., Petrucelli, L., Weng, Z., and Gao, F.-B. (2015). Differential Toxicity of Nuclear RNA Foci versus Dipeptide Repeat Proteins in a Drosophila Model of C9ORF72 FTD/ALS. Neuron 87, 1207-1214.
Ugolino, J., Ji, Y. J., Conchina, K., Chu, J., Nirujogi, R. S., Pandey, A., Brady, N. R., Hamacher-Brady, A., and Wang, J. (2016). Loss of C9orf72 Enhances Autophagic Activity via Deregulated mTOR and TFEB Signaling. PLoS Genet 12, e1006443.
van Blitterswijk, M., Gendron, T. F., Baker, M. C., DeJesus-Hernandez, M., Finch, N. A., Brown. P. H., Daughrity, L. M., Murray, M. E., Heckman, M. G., Jiang, J., et al. (2015). Novel clinical associations with specific C9ORF72 transcripts in patients with repeat expansions in C9ORF72. Acta neuropathologica 130, 863-876.
Wang, T., Liu, H., Itoh, K., Oh, S., Zhao, L., Murata, D., Sesaki, H., Hartung, T., Na, C. H., and Wang, J. (2021). C9orf72 regulates energy homeostasis by stabilizing mitochondrial complex I assembly. Cell metabolism 33, 531-546.
Warrell, R. P., Jr., He, L. Z., Richon, V., Calleja, E., and Pandolfi, P. P. (1998). Therapeutic targeting of transcription in acute promyclocytic leukemia by use of an inhibitor of histone deacetylase. J Natl Cancer Inst 90, 1621-1625.
Xi, Z., Zinman, L., Moreno, D., Schymick, J., Liang, Y., Sato, C., Zheng, Y., Ghani, M., Dib, S., Keith, J., et al. (2013). Hypermethylation of the CpG island near the G4C2 repeat in ALS with a C9orf72 expansion. Am J Hum Genet 92, 981-989.
Xie, L., Dong, P., Chen, X., Hsieh, T. S., Banala, S., De Marzio, M., English, B. P., Qi, Y., Jung, S. K., Kieffer-Kwon, K. R., et al. (2020). 3D ATAC-PALM: super-resolution imaging of the accessible genome. Nat Methods 17, 430-436.
Yang, L., Gal, J., Chen, J., and Zhu, H. (2014). Self-assembled FUS binds active chromatin and regulates gene transcription. Proc Natl Acad Sci USA 111, 17809-17814.
Yang, M., Liang, C., Swaminathan, K., Herrlinger, S., Lai, F., Shiekhattar, R., and Chen, J. F. (2016). A C9ORF72/SMCR8-containing complex regulates ULK1 and plays a dual role in autophagy. Sci Adv 2, e1601167.
Zhang, Y.-J., Guo, L., Gonzales, P. K., Gendron, T. F., Wu, Y., Jansen-West, K., O'Raw, A. D., Pickles, S. R., Prudencio, M., Carlomagno, Y., et al. (2019). Heterochromatin anomalies and double-stranded RNA accumulation underlie C9orf72 poly(PR) toxicity. Science 363, eaav2606.
Zhu, Q., Jiang, J., Gendron, T. F., McAlonis-Downes, M., Jiang, L., Taylor, A., Diaz Garcia, S., Ghosh Dastidar, S., Rodriguez, M. J., King, P., et al. (2020). Reduced C9ORF72 function exacerbates gain of toxicity from ALS/FTD-causing repeat expansion in C9orf72. Nat Neurosci 23, 615-624.
Zu, T., Liu, Y., Banez-Coronel, M., Reid, T., Pletnikova, O., Lewis, J., Miller, T. M., Harms, M. B., Falchook, A. E., Subramony, S. H., et al. (2013). RAN proteins and RNA foci from antisense transcripts in C9ORF72 ALS and frontotemporal dementia. Proc Natl Acad Sci USA 110, E4968-4977.

1-32. (canceled)

33. A method of treating a subject having a neurodegenerative disease, the method comprising administering to the subject an effective amount of a composition that decreases histone methylation and/or increases histone acetylation.

34. The method of claim 33, further comprising detecting increased DAXX amount or activity, histone hypermethylation, and/or histone hypoacetylation in a sample from the subject.

35. The method of claim 33, further comprising detecting a hexanucleotide repeat expansion in the C9orf72 locus of the subject.

36. The method of claim 33, wherein said composition comprises a DAXX inhibitor.

37. The method of claim 33, wherein said subject comprises a cell comprising a hexanucleotide repeat in chromosome 9.

38. The method of claim 33, wherein said subject comprises a cell comprising a hexanucleotide repeat at a C9orf72 locus.

39. The method of claim 33, wherein said subject comprises a cell comprising one or more GGGGCC repeats at a C9orf72 locus.

40. The method of claim 33, further comprising detecting normal histone methylation and/or normal histone acetylation in the subject after said administering.

41. The method of claim 33, further comprising detecting in the subject after said administering:
a decrease in histone methylation relative to a pre-treatment amount of histone methylation; and/or
an increase in histone acetylation relative to a pre-treatment amount of histone acetylation.

42. The method of claim 33, further comprising detecting in the subject after said administering a decrease in DAXX amount or activity relative to a pre-treatment amount of DAXX amount or activity.

43. The method of claim 33, further comprising administering to the subject a second effective amount of the composition.

44. The method of claim 33, wherein the subject is a human.

45. The method of claim 33, wherein the subject has amyotrophic lateral sclerosis (ALS), frontotemporal degeneration (FTD), Alzheimer's disease, Huntington's disease, multiple system atrophy, depressive pseudodementia, or bipolar disorder.

46. The method of claim 33, wherein said composition comprises 5-aza-2′-deoxycytidine (decitabine) or sodium phenylbutyrate.

47. The method of claim 33, wherein said composition comprises a compound comprising a structure according to:

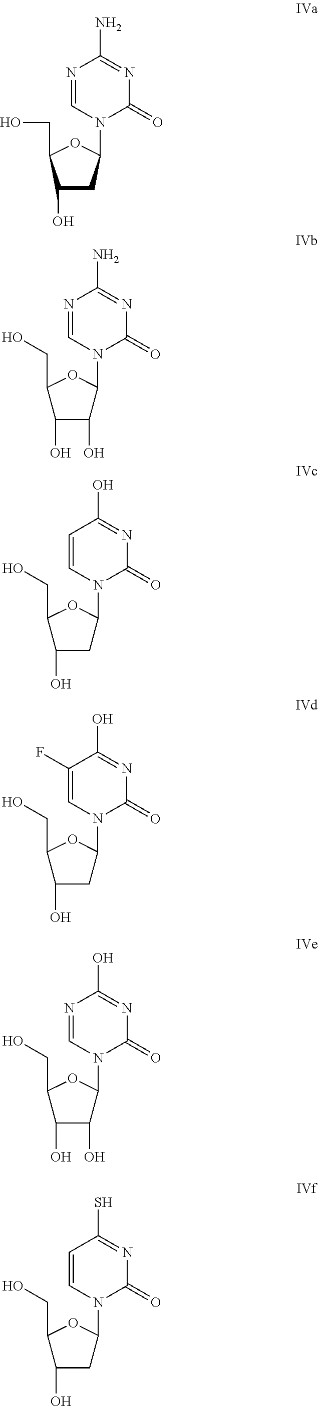

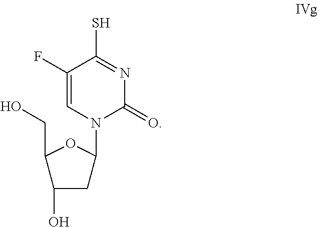 [SMILES: O=c1nc(S)c(F)cn1C1CC(O)C(CO)O1]

48. The method of claim 33, wherein said composition comprises a compound comprising a structure according to:

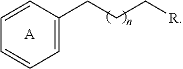

49. The method of claim 48, wherein A comprises one of:

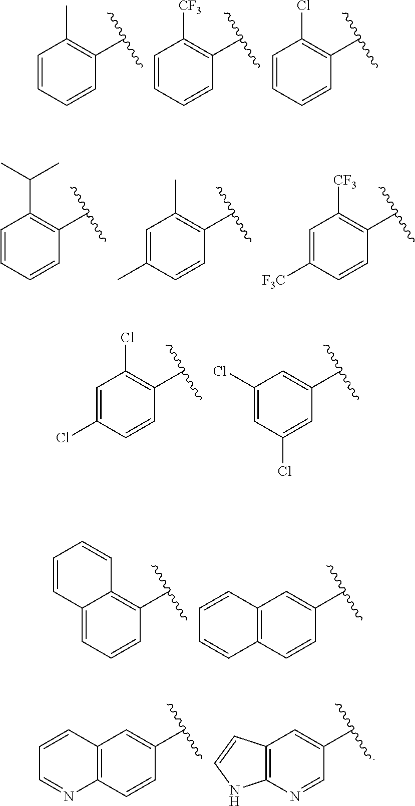

50. The method of claim 48, wherein n=0, 1, 2 3, 4, or 5.

51. The method of claim 48, wherein R comprises CO₂Na or one of: 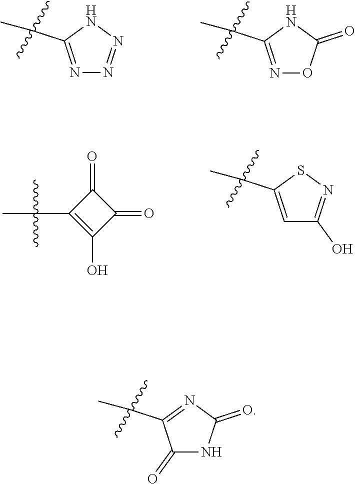 [SMILES: *c1nnn[nH]1 ; *c1noc(=O)[nH]1 ; *c1c(O)c(=O)c1=O ; *c1cc(O)ns1 ; *C1=NC(=O)NC1=O]


SEQUENCE LISTING (34 sequences)

SEQ ID NO: 1 - DNA, 24 bp, synthetic construct
  source 1..24: mol_type = other DNA
ccacgtaaaagatgacgcttgata

SEQ ID NO: 2 - DNA, 20 bp, synthetic construct
  source 1..20: mol_type = other DNA
cggtggcgagtggatatctc

SEQ ID NO: 3 - DNA, 20 bp, synthetic construct
  source 1..20: mol_type = other DNA
gcaagagcaggtgtgggttt

SEQ ID NO: 4 - DNA, 20 bp, synthetic construct
  source 1..20: mol_type = other DNA
tgggcaaagagtcgacatca

SEQ ID NO: 5 - DNA, 20 bp, synthetic construct
  source 1..20: mol_type = other DNA
tctggaactcaggagtcgcg

SEQ ID NO: 6 - DNA, 20 bp, synthetic construct
  source 1..20: mol_type = other DNA
gcagccgaaccccaaacagc

SEQ ID NO: 7 - DNA, 20 bp, synthetic construct
  source 1..20: mol_type = other DNA
tggcgagtgggtgagtgagg

SEQ ID NO: 8 - DNA, 23 bp, synthetic construct
  source 1..23: mol_type = other DNA
gagggaaagtaaaaatgcgtcga

SEQ ID NO: 9 - DNA, 22 bp, synthetic construct
  source 1..22: mol_type = other DNA
cacctatctcagcgatctgtct

SEQ ID NO: 10 - DNA, 20 bp, synthetic construct
  source 1..20: mol_type = other DNA
gttgcaggaccacttctgcg

SEQ ID NO: 11 - DNA, 20 bp, synthetic construct
  source 1..20: mol_type = other DNA
ccggaaaggaagaatatgga

SEQ ID NO: 12 - DNA, 24 bp, synthetic construct
  source 1..24: mol_type = other DNA
catttattgtttgatgttcactgc

SEQ ID NO: 13 - DNA, 21 bp, synthetic construct
  source 1..21: mol_type = other DNA
aaggtgaaggtcggagtcaac

SEQ ID NO: 14 - DNA, 22 bp, synthetic construct
  source 1..22: mol_type = other DNA
ggggtcattgatggcaacaata

SEQ ID NO: 15 - DNA, 24 bp, synthetic construct
  source 1..24: mol_type = other DNA
ccccggccccggccccggccccgg

SEQ ID NO: 16 - DNA, 24 bp, synthetic construct
  source 1..24: mol_type = other DNA
ccccggccccggccccggccccgg

SEQ ID NO: 17 - DNA, 60 bp, synthetic construct
  source 1..60: mol_type = other DNA
ggggccggggccggggccggggccggggccggggccggggccggggccggggccggggcc

SEQ ID NO: 18 - DNA, 60 bp, synthetic construct
  source 1..60: mol_type = other DNA
ggccccggccccggccccggccccggccccggccccggccccggccccggccccggcccc

SEQ ID NO: 19 - DNA, 60 bp, synthetic construct
  source 1..60: mol_type = other DNA
gactgactgatagatcctaagtactgattactgactatagatctaagtcatgatcagtta

SEQ ID NO: 20 - DNA, 60 bp, synthetic construct
  source 1..60: mol_type = other DNA
taactgatcatgacttagatctatagtcagtaatcagtacttaggatctatcagtcagtc

SEQ ID NO: 21 - DNA, 42 bp, synthetic construct
  source 1..42: mol_type = other DNA
ggggccggggccggggccggggccggggccggggccaacaac

SEQ ID NO: 22 - DNA, 42 bp, synthetic construct
  source 1..42: mol_type = other DNA
gttgttggccccggccccggccccggccccggccccggcccc

SEQ ID NO: 23 - DNA, 42 bp, synthetic construct
  source 1..42: mol_type = other DNA
gactgactgatagatcctaagtactgattactgactaacaac

SEQ ID NO: 24 - DNA, 42 bp, synthetic construct
  source 1..42: mol_type = other DNA
gttgttagtcagtaatcagtacttaggatctatcagtcagtc

SEQ ID NO: 25 - DNA, 53 bp, synthetic construct
  source 1..53: mol_type = other DNA
caagcagaagacggcatacgagataggagtccgtctcgtgggctcggagatgt

SEQ ID NO: 26 - DNA, 53 bp, synthetic construct
  source 1..53: mol_type = other DNA
acatctccgagcccacgagactaaggcgaatctcgtatgccgtcttctgcttg

SEQ ID NO: 27 - DNA, 53 bp, synthetic construct
  source 1..53: mol_type = other DNA
acatctccgagcccacgagaccgtactagatctcgtatgccgtcttctgcttg

SEQ ID NO: 28 - DNA, 53 bp, synthetic construct
  source 1..53: mol_type = other DNA
acatctccgagcccacgagacggactcctatctcgtatgccgtcttctgcttg

SEQ ID NO: 29 - DNA, 53 bp, synthetic construct
  source 1..53: mol_type = other DNA
acatctccgagcccacgagactaggcatgatctcgtatgccgtcttctgcttg

SEQ ID NO: 30 - DNA, 53 bp, synthetic construct
  source 1..53: mol_type = other DNA
acatctccgagcccacgagacctctctacatctcgtatgccgtcttctgcttg

SEQ ID NO: 31 - DNA, 53 bp, synthetic construct
  source 1..53: mol_type = other DNA
acatctccgagcccacgagaccagagaggatctcgtatgccgtcttctgcttg

SEQ ID NO: 32-33 - skipped (2 entries)

SEQ ID NO: 34 - DNA, 42 bp, synthetic construct
  source 1..42: mol_type = other DNA
gttgttagtcagtaatcagtagttaggatctatcagtcagtc